(19) United States
(12) Patent Application Publication
(10) Pub. No.: US 2021/0100897 A1
(43) Pub. Date: Apr. 8, 2021

(54) METHODS FOR THE STIMULATION OF DENDRITIC CELL (DC) PRECURSOR POPULATION "PRE-DC" AND THEIR USES THEREOF

(71) Applicant: AGENCY FOR SCIENCE, TECHNOLOGY AND RESEARCH, Singapore (SG)

(72) Inventors: Florent GINHOUX, Singapore (SG); Chi Ee Peter SEE, Singapore (SG)

(21) Appl. No.: 16/610,461

(22) PCT Filed: May 3, 2018
(86) PCT No.: PCT/SG2018/050219

§ 371 Date: Nov. 1, 2019

(30) Foreign Application Priority Data
May 3, 2017 (SG) 10201703628W

(51) Int. Cl. A61K 39/39; A61K 39/00; A61K 35/15; C12N 5/0784

(52) U.S. Cl. CPC A61K 39/39; A61K 39/0005; A61K 2039/5154; C12N 5/0639; A61K 2039/55561; A61K 35/15

(57) ABSTRACT

The present invention relates to a method of treating or preventing an infection, a neoplastic disease or an immune-related disease in a subject in need thereof, the method comprising contacting a therapeutically effective or immuno-effective amount of an TLR9 agonist, specifically CpG oligodeoxynucleotide 2216 (CpG ODN), with a precursor dendritic cell (pre-DC), wherein the TLR9 agonist stimulates the pre-DC to secrete one or more cytokines such as TNF-alpha and IL-12p40, to thereby activate or increase the subject's immune response for treating or preventing the infection, the neoplastic disease or the immune-related disease. The present invention also relates to immunogenic or adjuvant compositions comprising the TLR9 agonist. A method of diagnosing a deficient immune system in a subject, comprising contacting a sample comprising pre-DC from the subject with one or more TLR 9 agonists and kits thereof are also disclosed.

### TECHNICAL FIELD

The present invention generally relates to methods for stimulating pre-DC to increase the immune response for treating or preventing certain diseases in a subject in need thereof. The present invention also relates to molecules that are capable of effectively stimulating pre-DC to increase a subject's immune response, and molecules that are capable of being effective indicators of pre-DC stimulation and activation. The present invention further relates to an immunogenic composition for treating or preventing diseases or improving immunization by targeting pre-DC for an increased immune response.

### BACKGROUND

Dendritic cells (DC) are professional pathogen-sensing and antigen-presenting cells that are central to the initiation and regulation of immune responses. The DC population is classified into two lineages: plasmacytoid DC (pDC), and conventional DC (cDC), the latter comprising cDC1 and cDC2 sub-populations.

Both pDC and cDC arise from DC restricted bone-marrow (BM) progenitors known as common DC progenitors (CDP). Along the differentiation pathway of CDP giving rise to cDC, from BM to peripheral blood, it is believed that there is an intermediate population of cells called the precursor of cDC (pre-DC). The pre-DC compartment contains distinct lineage committed sub-populations including one early uncommitted CD123^{high} pre-DC subset and two CD45RA⁺CD123^{low} lineage-committed subsets called pre-cDC1 and pre-cDC2, which exhibit functional differences. Pre-cDC1 and pre-cDC2 eventually differentiate into cDC1 and cDC2, respectively.

The heterogeneous DC population is capable of processing and presenting antigens to naïve T cells to initiate antigen-specific immune responses. In many cases, increasing immune response to combat certain diseases is necessary to achieve desirable therapeutic effects. The conventional way of manipulating DC to increase immune responses in a subject includes stimulating various receptors expressed on the surface of DC. However, conventionally-defined pDC population is heterogeneous, incorporating an independent pre-DC sub-population. This makes it difficult to target specific populations of cells within the heterogeneous population to treat specific diseases. In addition, there is limited understanding of the pre-DC sub-population functions, especially the role of pre-DC in eliciting and increasing immune responses. Also, there has been no development of pre-DC specific therapeutic interventions, for example, in vaccines or treatment of diseases.

There is a need to provide means for stimulating pre-DC to increase the immune response for treating or preventing certain diseases in a subject in need thereof, that overcomes, or at least ameliorates, one or more of the disadvantages described above.

There is also a need to provide molecules which are capable of effectively stimulating pre-DC to activate or increase a subject's immune response, and molecules which are capable of being effective indicators of pre-DC stimulation and activation.

There is further a need to provide an immunogenic composition for treating or preventing diseases or improving immunization by targeting pre-DC for an increased immune response.

### SUMMARY

According to a first aspect, there is provided a method of treating or preventing an infection, a neoplastic disease or an immune-related disease in a subject in need thereof, the method comprising contacting a therapeutically effective or immuno-effective amount of an TLR9 agonist with a precursor dendritic cell (pre-DC), wherein the TLR9 agonist stimulates the pre-DC to secrete one or more cytokines, to thereby activate or increase the subject's immune response for treating or preventing the infection, the neoplastic disease or the immune-related disease.

According to a second aspect, there is provided use of one or more TLR9 agonists in the manufacture of a medicament for treating or preventing an infection, a neoplastic disease or an immune-related disease in a subject in need thereof, wherein the TLR9 agonist stimulates pre-DC to secrete one or more cytokines to thereby activate or increase the subject's immune response for treating or preventing the infection, the neoplastic disease or the immune-related disease.

According to a third aspect, there is provided an immunogenic composition comprising one or more TLR9 agonists capable of stimulating pre-DC to secrete one or more cytokines.

According to a fourth aspect, there is provided an adjuvant composition comprising a TLR9 agonist that is capable of stimulating pre-DC to secrete one or more cytokines for increasing a subject's immune response to treat or prevent an infection, a neoplastic disease or an immune-related disease.

According to a fifth aspect, there is provided a method of diagnosing a deficient immune system in a subject, said method comprising:

(a) obtaining a sample comprising pre-DC from the subject;
(b) contacting the sample with one or more TLR9 agonists;
(c) detecting the presence or absence of one or more cytokines in the sample; and
(d) diagnosing the subject as one having a deficient immune system when the one or more cytokines in the sample is absent (or not detected) or is present in a lower level when compared to a control sample.

According to a sixth aspect, there is provided a method of eliciting an immune response against an infection, a neoplastic disease or an immune-related disease in a subject in need thereof, the method comprising contacting an immuno-effective amount of an TLR9 agonist with pre-DC, wherein the TLR9 agonist stimulates the pre-DC to secrete one or more cytokines, to thereby elicit an immune response against the infection, the neoplastic disease or the immune-related disease.

According to a seventh aspect, there is provided a kit for diagnosing a deficient immune system in a subject according to the method as described herein.

### Definition of Terms

The following words and terms used herein shall have the meaning indicated:

The term “marker” refers to any biological compound, such as a protein and a fragment thereof, a peptide, a polypeptide, or other biological material whose presence, absence, level or activity is correlative of or predictive of a characteristic such as a cell type. Such specific markers may be detectable by using methods known in the art, such as but are not limited to, flow cytometry, fluorescent microscopy, immunoblotting, RNA sequencing, gene arrays, mass spectrometry, mass cytometry (Cy TOF) and PCR methods. A marker may be recognized, for example, by an antibody (or an antigen-binding fragment thereof) or other specific binding protein(s). Reference to a marker may also include its isoforms, preforms, mature forms, variants, degraded forms thereof (such as fragments thereof), and metabolites thereof.

The term “treatment” and variations of that term includes any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment.

The term “preventing” a disease refers to inhibiting completely or in part the development or progression of a disease (such as an immune-related disease) or an infection (such as an infection by a virus or bacteria). Vaccination is a common medical approach to prevent diseases where upon vaccination, immunization is initiated such that the body's own immune system is stimulated to protect the subject from infection or disease, or from subsequent infection or disease. Immunization may, for example, enable a continuing high level of antibody and/or cellular response in which T-lymphocytes can kill or suppress the pathogen in the immunized subject. The pathogen may be one which the subject has been previously exposed to.

The term “subject” refers to patients of human or other mammals, and includes any individual it is desired to be treated using the immunogenic compositions and methods of the disclosure. However, it will be understood that “subject” does not imply that symptoms are present. Suitable mammals that fall within the scope of the disclosure include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

The term “contacting” and variations of that term including “contact”, refers to incubating or otherwise exposing a compound or composition of the disclosure to cells (such as the pre-DC cells) of an organism (such as a subject as described herein). The contacting may occur in vitro, in vivo or ex vivo. The term “contacting” may also refer to administration of a compound or composition of the disclosure to an organism (such as a subject as described herein) by any appropriate means as described below.

The term “in vitro” as used herein refers to conducting a process or procedure outside a living organism, such as in a test tube, a culture vessel or a plate, or elsewhere outside the living organism.

The term “in vivo” as used herein refers to a process or procedure which is being performed in a subject.

The term “ex vivo” as used herein refers to a process or procedure conducted on live isolated cells outside a subject, and then returned to the living subject. For example, pre-DC may be extracted from a subject, contacted with a TLR9 agonist (for example, in a test tube, a culture vessel or a plate), and then returned to the subject to induce an immune response.

The term “administering” and variations of that term including “administer” and “administration”, includes contacting, applying, delivering or providing a compound or composition of the disclosure to an organism (such as a subject as described herein), or a surface by any appropriate means.

The term “immunogenic composition” as used herein refers to a composition which is capable of stimulating the immune system of a subject to produce an immune response. An immunogenic composition may comprise, for example, a specific type of antigen against which an immune response is desired to be elicited.

“Immune response” refers to conditions associated with, or caused by, inflammation, trauma, immune disorders, or infectious or genetic disease, and can be characterized by expression of various factors, e.g., cytokines, chemokines, and other signaling molecules, which may affect cellular and systemic defense systems.

The term “agonist”, when used in reference to TLR9, refers to a molecule which intensifies or mimics the biological activity of TLR9. Agonists may include proteins, nucleic acids, carbohydrates, small molecules, or any other compounds or compositions which modulate the activity of TLR9, either by directly interacting with TLR9 or by acting as components of the biological pathways in which TLR9 participates.

The term “antigen” refers to a molecule or a portion (such as a fragment) of a molecule capable of being recognized by antigen-binding molecules of the immune system, and inducing an immune response in the subject. Sources of antigen may be, but are not limited to, toxins, pollen, bacteria (or parts thereof), viruses (or parts thereof) or other microorganisms (or parts thereof). Parts of bacteria, viruses or other microorganisms which may act as antigens may be, but are not limited to, coats, capsules, cell walls, flagella, and fimbriae. If an antigen causes a specific disease (such as a disease caused by the host bacteria, virus or other microorganism which is the source of the antigen), then the antigen may be said to be associated with the disease.

Unless specified otherwise, the terms “comprising” and “comprise”, and grammatical variants thereof, are intended to represent “open” or “inclusive” language such that they include recited elements but also permit inclusion of additional, unrecited elements.

Throughout this disclosure, certain examples may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain examples may also be described broadly and generically herein. Each of the narrower species and subgeneric groupings falling within the generic disclosure also form part of the disclosure. This includes the generic description of the examples with a proviso or negative limitation removing any subject matter from the genus, regardless of whether or not the excised material is specifically recited herein.

### DETAILED DISCLOSURE OF THE EMBODIMENT

According to a first aspect, there is provided a method of treating or preventing an infection, a neoplastic disease or an immune-related disease in a subject in need thereof, the method comprising contacting a therapeutically effective or immuno-effective amount of an TLR9 agonist with a precursor dendritic cell (pre-DC), wherein the TLR9 agonist stimulates the pre-DC to secrete one or more cytokines, to thereby activate or increase the subject's immune response for treating or preventing the infection, the neoplastic disease or the immune-related disease. In one example, the pre-DC presents an antigen (or a fragment thereof) associated with the infection, the neoplastic disease or the immune-related disease in the subject. In another example, the pre-DC does not present any antigen. In one example, pre-DC were found to produce significantly more of the cytokines TNF-α and IL-12p40 when exposed to CpG ODN 2216 (also referred to as CpG, a TLR9 agonist), than either LPS (a TLR4 agonist) or polyI:C (TLR3 agonist)(see FIG. 5C). Cytokines such as TNF-α are known to exert a variety of effects on the immune response of a host such as in controlling infection and to modulate macrophage activity to control disease pathology. TNF-α has also been previously shown to exert a variety of effects in controlling infection. IL-12p40, another cytokine, is known to have protective function during infections. Thus, the contacting of TLR9 agonist with pre-DC enables a subject's immune response to be stimulated through the release of TNF-α and IL-12p40 cytokines to a therapeutically effective or immune-effective level for treating and preventing infections, neoplastic diseases or immune-related diseases.

Dendritic cells, such as pre-DC, are involved in the initiation of immune response to bacterial and viral infections. Upon infection by a pathogenic bacteria or virus, dendritic cells, such as pre-DC, will take up the bacterial or viral antigens in the peripheral tissues, process the antigens into proteolytic peptides, and load these peptides onto major histocompatibility complex (MHC) class I and II molecules. The dendritic cells, such as pre-DC, then become competent to present antigens to T lymphocytes, thus initiating antigen-specific immune responses. During this immune response, the TLR-9 agonist functions to specifically stimulate pre-DC to release cytokines to activate and/or enhance the immune response against the antigens.

Exemplary diseases in which the method as disclosed herein may be useful include but are not limited to bacterial infections, and viral infections, or the like. Examples of viruses which may cause viral infections are DNA viruses, and RNA viruses. Examples of DNA viruses are herpes simplex virus (HSV-1), cytomegalovirus (CMV), adenovirus, poxvirus, hepatitis B virus (HBV), or the like. Examples of RNA viruses are human immunodeficiency virus (HIV), hepatitis A virus (HAV), hepatitis C virus (HCV), respiratory syncytial virus (RSV), influenza, Zika virus, or the like.

In one example, the immune-related disease is an inflammatory disease. In another example, the immune-related disease is an autoimmune disease. Immune-related diseases may be caused by dysfunction or abnormality in the immune response. The dysfunction or abnormality in the immune response may be caused by genetic mutations, reaction to a drug, radiation therapy, or other chronic and/or serious disorders (such as cancer or diabetes).

In one example, the autoimmune disease is selected from the group consisting of systemic lupus erythematosus (SLE) and Sjögren's syndrome.

Exemplary TLR9 agonists which may be useful for stimulating the pre-DC cells include but is not limited to an oligodeoxynucleotides (ODN), or a biological or functional variant thereof.

Exemplary CpG oligodeoxynucleotides include CpG ODN Class A, CpG ODN Class B and CpG ODN Class C. In one example, the CpG oligodeoxynucleotide is CpG ODN 2216, or a biological or a functional variant thereof.

The biological variant of a CpG ODN is expected to display substantially the same biological activity as the CpG ODN 2216 of which it is a variant. For example, the biological variant of CpG ODN 2216 is expected to display substantially the same biological activity as CpG ODN 2216 as an agonist of TLR9. Alternatively, the TLR9 agonist may be a functional variant of a CpG ODN. A functional variant typically has substantial or significant sequence identity or similarity to the CpG ODN of which it is a variant, such as at least 80% (e.g. 80%, 85%, 90%, 91%, 92%, 93%, 94%, 95%, 96%, 97%, 98%, or 99%) identity to the CpG ODN sequence of which it is a variant, and retains the same activity as the CpG ODN.

The TLR9 agonist (or a composition thereof) may be contacted with a pre-DC or administered in a therapeutically effective amount or an immune-effective amount. A therapeutically effective amount includes a sufficient but non-toxic amount of a TLR9 agonist (or a composition thereof) to provide the desired therapeutic effect. An immune-effective amount includes a sufficient but non-toxic amount of a TLR9 agonist (or a composition thereof) to provide the desired immunoprotective effect. The exact amount required will vary from subject to subject depending on factors such as the species being treated, the age and general condition of the subject, the severity of the condition being treated, the particular agent or composition being contacted or administered, the mode of contact or administration, and so forth. Thus, it is not possible to specify an exact “effective amount”. However, for any given case, an appropriate “effective amount” may be determined by one of ordinary skill in the art using only routine experimentation. For example, an effective amount to result in therapeutic or immunoprotective amount may be an amount sufficient to result in the improvement of the pathological symptoms of a target disease or an amount sufficient to result in protection against a target infectious disease. Generally, an effective dosage may be in the range of about 100 ng/kg to about 100 mg/kg, about 100 ng/kg to about 90 mg/kg, about 100 ng/kg to about 80 mg/kg, about 100 ng/kg to about 70 mg/kg, about 100 ng/kg to about 60 mg/kg, about 100 ng/kg to about 50 mg/kg, about 100 ng/kg to about 40 mg/kg, about 100 ng/kg to about 30 mg/kg, about 100 ng/kg to about 20 mg/kg, about 100 ng/kg to about 10 mg/kg, about 90 ng/kg to about 100 mg/kg, about 80 ng/kg to about 100 mg/kg, about 70 ng/kg to about 100 mg/kg, about 60 ng/kg to about 100 mg/kg, about 50 ng/kg to about 100 mg/kg, about 40 ng/kg to about 100 mg/kg, about 30 ng/kg to about 100 mg/kg, or about 20 ng/kg to about 100 mg/kg, and includes any subranges therein, as well as individual numbers within the ranges and subranges.

Exemplary cytokines which may be produced by pre-DC upon stimulation with a TLR9 agonist include but are not limited to tumor necrosis factors, interleukins, interferons, and chemokines, or the like.

In one example, the tumor necrosis factor that is produced by pre-DC upon stimulation with a TLR9 agonist is TNF-α. In one example, CpG ODN 2216 was shown to stimulate pre-DC to produce high levels of cytokine, specifically TNF-α (see FIG. 5C).

In another example, the interleukin that is produced by pre-DC upon stimulation with a TLR9 agonist is IL-12p40. In one example, IL-12p40 was shown to be readily secreted by pre-DC when stimulated with TLR9 agonists (see FIG. 2G).

In yet another example, the interferon that is produced by pre-DC upon stimulation with a TLR9 agonist is IFN-α.

Pre-DC is a subset of CD33⁺CD45RA⁺CD123⁺ cell which gives rise to cDC subsets (FIG. 2A, and FIG. 10A). Pre-DC cells also express CX3CR1, CD2, CD303 and CD304, with low CD11c expression (FIGS. 2, A and B, and FIGS. 10, B and C). The pre-DC may be identified based on the expression of pre-DC-specific marker genes such as those listed in FIG. 27 and FIG. 28. For example, the pre-DC may be isolated based on the specific marker genes through conventional gating strategy such as, but not limited to those, described in FIGS. 10A, 11, 12A-C, 14, 15, 18 and 19.

In another example, the pre-DC comprises one or more markers selected from the group consisting of CD123, CD303, CD304, CD327, CD45RA, CD85j, CD5 and BTLA. The expression of the markers may be determined based on the gene expression or protein expression levels using methods known in the art, such as but are not limited to, flow cytometry, fluorescent microscopy, immunoblotting, RNA sequencing, gene arrays, mass spectrometry, mass cytometry (Cy TOF) and PCR methods.

Early pre-DC can differentiate to both cDC subsets, and committed pre-DCs such as pre-conventional dendritic cells 1 (pre-cDC1) and pre-conventional dendritic cells 2 (pre-cDC2) differentiate exclusively into cDC1 and cDC2 subsets, respectively (FIG. 3H, FIG. 18D, and FIG. 19).

Therefore, in one example, the pre-DC is selected from the group consisting of early pre-DC, pre-conventional dendritic cells 1 (pre-cDC1), and pre-conventional dendritic cells 2 (pre-cDC2).

In one example, the subject is a human. The subject may be one suffering from any of the diseases disclosed herein and is in need of treatment. The subject may also be a human at risk of any of the bacterial or viral infections disclosed herein, such as subjects living in (or in close proximity to areas) with a bacterial or viral outbreak who may require vaccination against these infections. The human subjects can be either adults or children. In another example, the subject is a human suffering from any of the immune-related disease disclosed herein. In yet another example, the subject is a human with a deficient immune system. The methods of the disclosure can also be used on other subjects at risk of any of the bacterial or viral infections disclosed herein or suffering from any of the diseases disclosed herein such as, but not limited to, non-human primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes).

The TLR9 agonist may be administered to the subject by any route suitable for administration of such compounds, such as, intramuscular, intradermal, subcutaneous, intravenous, oral, and intranasal administration. Thus, the TLR9 agonist of the disclosure may be in a formulation suitable for parenteral administration (that is, subcutaneous, intramuscular or intravenous injection), in the form of a formulation suitable for oral ingestion (such as capsules, tablets, caplets, elixirs, for example), or in an aerosol form suitable for administration by inhalation (such as by intranasal inhalation or oral inhalation).

For administration as an injectable solution or suspension, non-toxic parenterally acceptable diluents or carriers can include Ringer's solution, isotonic saline, phosphate buffered saline, ethanol and 1,2 propylene glycol.

For oral administration, suitable carriers, diluents, excipients and adjuvants include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Drops for oral administration according to the present disclosure may comprise sterile aqueous or oily solutions or suspensions. These may be prepared by dissolving the immunogenic agent in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Upon contact with the TLR9 agonist (or a composition comprising the TLR9 agonists described above) with pre-DC, the subject's immune response may be stimulated through the release of cytokines (such as, but not limited to, TNF-α and IL-12p40) to a therapeutically effective or immune-effective level for treating and preventing infections, neoplastic diseases or immune-related diseases.

According to a second aspect, there is provided use of one or more TLR9 agonists in the manufacture of a medicament for treating or preventing an infection, a neoplastic disease or an immune-related disease in a subject in need thereof, wherein the TLR9 agonist stimulates pre-DC that present an antigen (or a fragment thereof) associated with the infection or immune-related disease in the subject to secrete one or more cytokines to thereby increase the subject's immune response for treating or preventing the infection, the neoplastic disease or the immune-related disease.

In one example, the medicament is a vaccine for preventing an infection, a neoplastic disease or an immune-related disease in a subject in need thereof.

According to a third aspect, there is provided an immunogenic composition comprising: (a) an antigen (or a fragment thereof) associated with an infection, a neoplastic disease or an immune-related disease, and (b) one or more TLR9 agonists capable of stimulating pre-DC that present the antigen (or a fragment thereof) to secrete one or more cytokines.

As described herein, an immunogenic composition is a composition which is capable of stimulating the immune system of a subject to produce an immune response against an antigen. Sources of antigen may be, but are not limited to, toxins, pollen, bacteria (or parts thereof), viruses (or parts thereof) or other microorganisms (or parts thereof). Parts of bacteria, viruses or other microorganisms which may act as antigens may be, but are not limited to, coats, capsules, cell walls, flagella, and fimbriae.

In one example, the immunogenic composition is a vaccine.

In general, suitable immunogenic compositions may be prepared according to methods which are known to those of ordinary skill in the art and accordingly may include a pharmaceutically acceptable carrier, diluent and/or adjuvant. The carriers, diluents and adjuvants must be “acceptable” in terms of being compatible with the other ingredients of the composition, and not deleterious to the recipient thereof.

One skilled in the art would be able, by routine experimentation, to determine an effective and safe amount of the immunogenic composition for contact or administration to achieve the desired immunogenic response.

Generally, an effective dosage to achieve the desired immunogenic response is expected to be in the range of about 0.0001 mg to about 1000 mg per kg body weight per 24 hours; typically, about 0.001 mg to about 750 mg per kg body weight per 24 hours; about 0.0 1 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage to achieve the desired immunogenic response may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

According to a fourth aspect, there is provided an adjuvant composition comprising a TLR9 agonist that is capable of stimulating pre-DC that present an antigen (or a fragment thereof) associated with an infection, a neoplastic disease or an immune-related disease in a subject to secrete one or more cytokines for increasing the subject's immune response to treat or prevent the infection, the neoplastic disease or the immune-related disease.

As an adjuvant composition, the adjuvant composition comprising a TLR9 agonist is capable of increasing the effectiveness of a composition for stimulating immune response, for example through stimulation of cytokines release from pre-DC.

As described herein, in one example, the subject who may benefit from the methods or compositions of the disclosure is one who has a deficient immune system. A subject with deficient immune system may be one who is unable to activate the immune response, or one whose immune system is partially activated (for example, activated to only a certain extent, such as in the range of about 10% to about 90%, about 10% to about 80%, about 10% to about 70%, about 10% to about 60%, about 10% to about 50%, about 10% to about 40%, about 10% to about 30%, about 10% to about 20%, and includes any subranges therein, as well as individual numbers within the ranges and subranges, compared to a subject without a deficient immune system). Such a condition may be due to abnormal pre-DC cells which are unable to produce cytokines, resulting in a deficient level of cytokines required for activation of the immune response. For example, while a normal pre-DC is able to secrete cytokines, such as TNF-α and IL-12p40, when stimulated, a subject with abnormal pre-DC may secrete lower levels of cytokines (or no cytokines) compared to a healthy subject.

Therefore, according to a fifth aspect, there is provided a method of diagnosing a deficient immune system in a subject, said method comprising:

(a) obtaining a sample comprising pre-DC from the subject;
(b) contacting the sample with one or more TLR9 agonists;
(c) detecting the presence or absence of one or more cytokines in the sample; and
(d) diagnosing the subject as one having a deficient immune system when the one or more cytokines in the sample is absent (or not detected) or is present in a lower level when compared to a control sample.

Samples suitable for use in the methods described herein include tissue culture, blood, apheresis residue, tissue (from various organs, such as spleen, kidney, etc.), peripheral blood mononuclear cells or bone marrow. The samples may be obtained by methods, such as but not limited to, surgery, aspiration or phlebotomy. The samples may be untreated, treated, diluted or concentrated from the subject.

The contacting of the samples with one or more TLR9 may be conducted in vitro, in vivo or ex vivo.

The cytokines may be detected using methods known in the art, such as but are not limited to, labelling with cytokine-specific antibodies followed by flow cytometry analysis, ELISA, or other commercially available cytokine detection assay kits (such as the Luminex assay kits).

In the context of detecting cytokine, such as a TNF-α and IL-12p40, the term “absence” (or grammatical variants thereof) can refer to when cytokine cannot be detected using a particular detection methodology. For example, cytokine may be considered to be absent in a sample if the sample is free of cytokine, such as, 95% free, 96% free, 97% free, 98% free, 99% free, 99.9% free, or 100% free of cytokine, or is undetectable as measured by the detection methodology used. Alternatively, if the level of cytokine (such as TNF-α and IL-12p40) is below a previously determined cut-off level, the cytokine may also be considered to be “absent” from the sample.

In the context of detecting cytokine, such as a TNF-α and IL-12p40, the term “presence” can refer to when a cytokine can be detected using a particular detection methodology. For example, if the level of cytokine (such as TNF-α and IL-12p40) is above a previously determined threshold level, the cytokine may be considered to be “present” in the sample.

A control sample that may be used in the methods disclosed herein includes, but is not limited to, a sample which is not contacted with one or more TLR9 agonist or a sample from a healthy subject (for example, a subject whose immune system is not deficient) which has been contacted with one or more TLR9 agonist.

In one example, the method further comprises treating the subject diagnosed with a deficient immune system by administering a composition described herein, to thereby increase the subject's immune response.

According to a sixth aspect, there is provided a method of eliciting an immune response against an infection, a neoplastic disease or an immune-related disease in a subject in need thereof, the method comprising contacting an immuno-effective amount of an TLR9 agonist with a pre-DC, wherein the TLR9 agonist stimulates precursor dendritic cells (pre-DC) that present an antigen (or a fragment thereof) associated with the infection, the neoplastic disease or the immune-related disease in the subject to secrete one or more cytokines, to thereby elicit an immune response against the infection, the neoplastic disease or the immune-related disease.

The immune response may be considered “elicited” when the humoral and/or cell-mediated immune responses are triggered, resulting in protection of the subject from subsequent infections, removal of pathogenic bacteria, virus or microorganisms, and/or inhibition of the development or progression of a disease or infection by a virus or bacteria.

According to a seventh aspect, there is provided a kit for diagnosing a deficient immune system in a subject according to the method as described herein. Other components of a kit may include, but are not limited to, one or more of the TLR9 agonist described above, one or more cytokine-specific antibodies, one or more buffers, and one or more diluents.

### BRIEF DESCRIPTION OF THE DRAWINGS

The invention will be better understood with reference to the detailed description when considered in conjunction with the non-limiting examples and the accompanying drawings, in which:

FIG. 1. MARS-seq and CyTOF identify rare CD123⁺CD33⁺ putative DC precursors (pre-DC). (A-E) Lin(CD3/CD14/CD16/CD20/CD34)⁻HLA-DR⁺CD135⁺ sorted PBMC were subjected to MARS-seq. (A) shows a t-stochastic neighbor embedding (tSNE) plot of 710 cells fulfilling all quality criteria, displayed by clusters identified by tSNE plus Seurat clustering, or by the relative signature score for pDC, cDC1 and cDC2. (B) illustrates a connectivity MAP (cMAP) analysis showing the degree of enrichment for pDC or cDC signature genes in the tSNE/Seurat clusters. (C) shows Mpath analysis applied to the tSNE/Seurat clusters defining their developmental relationship. Representations of the 710 cells by (D) Monocle, (E) Principal component analysis (PCA) and (F) Diffusion Map, highlighting the tSNE/Seurat clusters identified in (A). (G) shows violin plots of tSNE/Seurat pDC clusters, cluster #4 and cDC clusters showing the expression of pDC and cDC signature genes with differential expression between cluster #4 and pDC clusters. Adjusted P-values were calculated by Kruskal-Wallis test followed by Dunn's multiple comparisons procedure. (H, I) provide tSNE plots of CyTOF data from CD45⁺Lin(CD7/CD14/CD15/CD16/CD19/CD34)⁻HLA-DR⁺PBMC, showing in (H) gates defining the CD123⁺CD33⁺ cells and DC subsets, and in (I) relative expression of selected markers. In (J), subsets defined in (H) were overlaid onto 2D-contour plots for phenotypic comparison. The gating strategy prior to MARS-seq is shown in FIG. 7A.

FIG. 2. Characterization of human pre-DC. (A) shows flow cytometric identification of pre-DC and pDC within PBMC and spleen cell suspensions. (B) shows expression of CD303/CD304/CD123/CD11c by blood pre-DC and DC subsets. (C) shows % pre-DC within spleen (n=3) and PBMC (n=6) CD45⁺ populations. (D) shows Wright-Giemsa staining of sorted blood pre-DC and DC subsets. (E) shows electron micrographs of pre-DC and pDC (RER (arrowheads), centriole (C) and microtubules (small arrows), near RER cisterna are indicated). (F) shows DC subsets or pre-DC co-cultured for 5 days with MS-5 feeder-cells, FLT3L, GM-CSF and SCF. Their capacity to differentiate into cDC1 or cDC2 was measured by flow cytometry. (n=3) (G) shows intracellular detection of cytokines in DC subsets and pre-DC post-TLR stimulation. IFN-α and IL-12p40 production by pDC and pre-DC, alongside mean % cytokine-positive pre-DC and DC subsets exposed to LPS, LPS+IFNγ (L+I), polyI:C (pI:C), CL097 (CL) or CpG-ODN2216 (CpG) (n=4) are shown. (H) shows the proliferation of naïve CD4⁺ T cells cultured for 6 days with allogeneic pDC, total CD123⁺HLA-DR⁺ cells or pre-DC (n=2). (I) shows frequency of pDC and pre-DC from control subjects (Ctrl, n=11) and Pitt-Hopkins Syndrome (PHS) patients (n=4). P-values were calculated by Mann-Whitney test. Error bars represent mean+/−SEM.

FIG. 3. Identification of committed human pre-DC subsets. (A-B) shows single-cell mRNA sequencing (scmRNAseq) of 92 Lin(CD3/14/16/19/20)⁻HLA-DR⁺CD33⁺CD123⁺ cells (sort gating strategy shown in FIG. 14A). (A) shows the connectivity MAP (cMAP) enrichment score of cells (cDC1- vs cDC2-specific signatures). (B) shows the Mpath analysis showing the developmental relationship between “unprimed”, cDC1-primed or cDC2-primed cells defined in (A). (C) shows Lin⁻HLA-DR⁺CD33⁺PBMC analyzed by flow cytometry and visualized as 3D-PCA of three cell clusters (pre-DC, cDC1 and cDC2) and the relative expression of CADM1, CD1c and CD123. (D) shows relative expression of CD45RA, BTLA, CD327, CD141 and CD5 in the same 3D-PCA plot. The dashed black circles indicate the intermediate CD45RA⁺ population. (E) shows CD45RA/CD123 dot plots showing overlaid cell subsets defined in the 3D-PCA plot (left panel) with the relative expression of BTLA, CD327, CD141 and CD5. (F) shows overlay of the Wanderlust dimension (progression from early (dark) to late (clear) events is shown) onto the 3D-PCA and CD45RA/CD123 dot plots. (G) illustrates the gating strategy starting from live CD45⁺Lin(CD3/14/16/19/20)⁻CD34⁻HLA-DR⁺PBMC to define pre-DC subsets among CD33⁺CD45RA⁺cDC. (H) shows pre-DC subsets co-cultured for 5 days with MS-5 feeder-cells, FLT3L, GM-CSF and SCF (n=3). Their capacity to differentiate into Clec9A⁺CADM1⁺cDC1 (middle panel), or CD1c⁺CD11c⁺cDC2 (right panel) was analyzed by flow cytometry. (I) shows scanning electron microscopy of pre-DC and DC subsets (scale bar: 1 μm).

FIG. 4. DC and pre-DC subset gene expression analysis. (A) shows microarray data from sorted DC and pre-DC subsets (shown in FIG. 3) were analyzed by 3D PCA using differentially-expressed genes (DEG). For each PCA dimension (principal component, PC), the variance explained by each component is indicated. (B-D) show heat maps of DEG between (B) early pre-DC/pDC, (C) early pre-DC/pre-cDC1/cDC1 and (D) early pre-DC/pre-cDC2/cDC2. (E) shows expression profiles of 62 common genes identified from DEG analysis comparisons along the lineage progression from early pre-DC to mature cDC, for cDC1 and cDC2 respectively. The profiles were plotted with the log 2 fold-change values (versus early pre-DC). (F) shows expression level of CD327 (SIGLEC6), CD22 and AXL proteins by DC and pre-DC subsets evaluated by flow cytometry. The mean fluorescence intensities are indicated. (G) shows expression profiles of selected transcription factors.

FIG. 5. Functional analysis of DC and pre-DC subsets. (A) shows frequency of cytokine production by pre-DC and DC subsets upon TLR stimulation measured by intracellular flow cytometry. Dot plots (left panel) show IFNα, IL-12p40 and TNF-α production by pDC, early pre-DC, pre-cDC2, cDC2, pre-cDC1 and cDC1. Bar charts (right panel) show the mean relative numbers of pre-DC and DC subset cells producing IFN-α, IL-12p40 or TNF-α in response to LPS, LPS+IFNγ (L+I), CL097 (CL) or CpG ODN2216 (CpG) (n=4). (B) shows expression level (represented as mean fluorescence intensity (MHO) of costimulatory molecules (CD40, CD80, CD83, CD86) by blood pre-DC and DC subsets (n=4). (C) shows proliferation of naïve CD4⁺ T cells after 6 days of culture with allogenic pre-DC and DC subsets (n=3). P-values were calculated by Mann-Whitney test. Error bars represent mean+1/−SEM.

FIG. 6. Unsupervised mapping of DC ontogeny using CyTOF. CyTOF data from bone marrow (BM) and PBMC were analyzed using isoMAP dimensionality reduction to compare overall phenotypic relatedness of cell populations, and were automatically subdivided into clusters using the phenograph algorithm. (A, B) show IsoMAP1-2 plots showing the expression level of common DC progenitor (CDP), pDC, pre-DC and cDC specific markers within (A) BM and (B) blood Lin(CD3/CD7/CD14/CD15/CD19/CD34)⁻ HLA-DR⁺CD123⁺ cells. (C) shows phenotypic association between Lin-HLA-DR⁺CD123^{hi} BM and CD123⁺PBMC, showing progression from CDP towards pDC or pre-DC in the BM, and the clear separation of pDC and pre-DC in the blood. Cells within the pre-DC phenograph clusters (clusters #1 and #2 in the BM, and #6 in the blood) and cells within the pDC phenograph clusters (clusters #3 and #4 in the BM, and #7 in the blood) were further analyzed by isoMAP to define pre-DC subsets (left panels, and FIGS. 26, C and D) and heterogeneity among pDC (right panels, and FIGS. 26, D and E).

FIG. 7. (A) shows gating strategy for FACS of single cells from total Lin⁻HLA-DR⁺CD135⁺ cells. (B) shows the workflow of the MARS-seq single cell data analysis. (C) shows the association between molecule counts and cells. Cell IDs were sorted from highest to lowest number of unique molecular identifier (UMI) or molecule counts. The data are presented on a log₁₀ axis. The three horizontal lines correspond to molecule counts of 650 (bottom), 1,050 (middle) and 1,700 (top) per cell. The shaded area indicates the range of molecule counts from 400 to 1,200 UMIs per cell. Cells with <1,050 molecules were removed from the analysis (n=1,786 cells). A total of 710 high-quality cells were used for further downstream analyses. (D) shows a density plot (top panel) representing the distribution of cells with a certain number of molecules, and the first (middle panel) and second derivative (bottom panel) of the density function. The three lines correspond to molecule counts of 650 (left), 1,050 (middle) and 1,700 (right) per cell. (E) shows principal component analysis (PCA) after simulation at different normalization thresholds. Points were colored according to the different runs. (F) shows a correlation plot of average expression of genes in run2 (y-axis) versus average expression of genes in run1 (x-axis). The data are presented on a log₁₀ axis. The Pearson correlation coefficient was 0.99. (G) shows t-distributed stochastic neighbor embedding (tSNE) analysis of the 710 single cells, colored by run association (run 1: dark, run 2: light), showed an even distribution of the cells within the tSNE plot. Lines represent a linear fit of the points. The distributions of the points along the tSNE component 1 and component 2 were represented as density plots on the top or right panel, respectively. (H) shows frequency of cells in the five determined clusters for run1 and run2. (I) shows that the mean-variability plot showed average expression and dispersion for each gene. This analysis was used to determine highly variable gene expression (labeled by gene symbol). The 36 highly variable genes were used to perform a dimensionality reduction of the single-cell data by PCA. In (J), the highest gene loadings in the first and second principal component (PC1 and PC2) from the PCA of 710 high quality cells are shown.

FIG. 8. (A) shows the relative expression of signature genes of pDC (TCF4), cDC1 (CADM1) and cDC2 (CD1D) in Mpath clusters defined in FIG. 1C. (B) shows the weighted neighborhood network of the Mpath analysis shown in FIG. 1C. (C) shows the analysis of MARS-seq data using the Wishbone algorithm. In the 2D-t-distributed stochastic neighbor embedding (tSNE) plot (upper panels) and in the 3D-Diffusion Map (lower panels) (See FIGS. 1, A and F, respectively), cells were displayed according to the values of the Wishbone trajectory (left panels) or the values of the Wishbone branches (right panels). Line chart (top right panel) shows the expression of signature genes along Wishbone trajectory. X-axis represents pseudo-time of Wishbone trajectory. Solid line represents backbone trajectory, dotted lines represent separate trajectories along the two branches. Heat maps (bottom right panels) show the expression of signature genes along Wishbone trajectory on the two branches.

FIG. 9. (A) shows the gating strategy of CD45⁺Lin(CD7/CD14/CD15/CD16/CD19/CD34)⁻HLA-DR⁺ blood mononuclear cells from CyTOF analysis for downstream t-distributed stochastic neighbor embedding (tSNE) as shown in FIG. 1, E to G. The name of the excluded population(s) is indicated in each corresponding 2D-plot. (B) shows tSNE plots of the CyTOF data from FIG. 1, H to J showing the expression level of cDC2-, cDC1- and pDC-specific markers. (C) shows that unsupervised phenograph clustering identified 10 clusters that were overlaid onto the tSNE1/2 plot of the CyTOF data from FIGS. 1, H and I.

FIG. 10. (A) shows the gating of flow cytometry data to identify the Lin⁻HLA-DR⁺ cell population displayed in FIG. 2A (blood data displayed). (B) shows classical contour plots of CyTOF data from FIG. 1 showing the same gating strategy as applied in the flow cytometry analyses shown in FIG. 2A. (C) shows flow cytometry data of the relative expression of CD33, CX3CR1, CD2, CD141, CD11c, CD135, CD1c and CADM1 by pre-DC, pDC, cDC1 and cDC2 defined in FIG. 2A in the blood (upper panels) and spleen (lower panels). (D) shows a ring graphical representation of the proportion of pre-DC, cDC1 and cDC2 among total Lin⁻CD34⁻HLA-DR⁺CD33⁺cDC defined in FIG. 2A in the spleen (left) and blood (right). (E) shows representative electron micrographs showing morphological characteristics of a pre-DC. (F) shows histograms of the mean relative numbers of CD123⁺CD172α⁻cells, Clec9A⁺CADM1⁺cDC1 or CD172α⁺CD1c⁺cDC2 from the in vitro differentiation assays as described in FIG. 2F (n=4). Error bars represent mean±SEM.

FIG. 11. Gating strategy for the fluorescence-activated cell sorting of DC subsets and pre-DC used in the in vitro differentiation assays (FIG. 2F). (A) shows pre-sorted data and (B-E) show post-sorted re-analysis of (B) pre-DC, (C) cDC1, (D) cDC2, and (E) pDC.

FIG. 12. (A)-(C) show comparison of (A) the gating strategy from Breton et al. (32) (Pre-DC are shown in the two plots on the top right.) and (B) the gating strategy used in FIG. 2A and FIG. 10A (pre-DC displayed in purple) to define pre-DC. The relative numbers of pre-DC defined using the two gating strategies among live CD45⁺ peripheral blood mononuclear cells are indicated in the dot plots. (C) shows graphical representation of the median relative numbers of pre-DC defined using the two gating strategies among live CD45⁺ blood mononuclear cells (n=4). The median percentages of CD45⁺ values are indicated. P-values were calculated using the Mann-Whitney test. (D) illustrates a histogram showing the expression of CD117 by DC subsets and pre-DC determined by flow cytometry. (E)-(F) show identification of pre-DC, cDC1 and cDC2 among Lin⁻HLA-DR⁺ (E) ILT3⁺ILT1⁻ cells (33) or ILT3⁺ILT1⁺ (cDC), and (F) CD4⁺CD11c⁻ cells (34) or CD4^{int}CD11c⁺cDC.

FIG. 13. shows pDC, pre-DC, cDC1 and cDC2 isolated by fluorescence-activated cell sorting were stimulated in vitro with LPS, LPS+IFNγ (L+I), Flagellin (Flag), polyI:C (pI:C), CL097 (CL) or CpG ODN2216 (CpG), and the soluble mediators (as indicated above each histogram) in the culture supernatants were quantified by Luminex Multiplex Assay (n=2).

FIG. 14. shows identification of CD33⁺CX3CR1⁺ pre-DC among Lin⁻HLA-DR⁺CD303⁺CD2⁺ cells (36).

FIG. 15. shows the gating strategy for the fluorescence-activated cell sorting analysis of peripheral blood mononuclear cells from control subjects (Ctrl, n=11) and patients with Pitt-Hopkins Syndrome (PHS; n=4). pDC (circled in blue) and pre-DC (circled in purple) were defined among Lin-HLA-DR⁺CD45RA⁺CD123⁺ cells.

FIG. 16. (A) shows the gating strategy for FACS of Lin⁻HLA-DR⁺CD33⁺CD45RA⁺CD1c^{lo/-}CD2⁺CADM1^{lo/-}CD123⁺ pre-DC analyzed by C1 single cell mRNA sequencing (scmRNAseq). (B) shows quality control (removing low-quality cells and minimally-expressed genes below the limits of accurate detection; low-quality cells that were identified using SINGuLAR toolbox; minimally-expressed genes with transcripts per million (TPM) values ≥1 in <95% of the cells) and (C) shows the work flow of the C1 scmRNAseq analyses shown in FIG. 3A-B. Error bars represent the maximum, third quartile, median, first quartile and minimum.

FIG. 17. shows the relative expression levels of signature genes of cDC1 (BTLA, THBD and, LY75) and cDC2 (CD2, SIRPA and ITGAX) in Mpath clusters defined in FIG. 3B.

FIG. 18. (A) shows the expression level of markers in the 3D-Principal Component Analysis (PCA) plots from FIGS. 3, C and D. (B) shows the sequential gating strategy of flow cytometry data starting from Live CD45⁺Lin(CD3/14/16/19/20)⁻CD34⁻HLA-DR⁺ peripheral blood mononuclear cells defining CD33⁻CD123⁺CD303⁺ pDC, CD33⁺CD45RA⁻differentiated cDC (CADM1⁺cDC1, CD1c⁺cDC2), and CD33⁺CD45RA⁺ cells (comprising CD123⁺CD45RA⁺ pre-DC and CD123^{lo}CD45RA⁺ intermediate cells). (C) shows the proportion of CD45⁺ mononuclear cells in spleen (n=3) (left) and peripheral blood (n=6) (right) of the above-mentioned pre-DC subsets. (D) shows histograms of the mean proportion of CD303⁺CD172α⁻cells, Clec9A⁺CADM1⁺cDC1 or CD1c⁺CD11c⁺cDC2 obtained in the in vitro differentiation assays as described in FIG. 3H (n=3). Error bars represent mean±SEM.

FIG. 19. shows the gating strategy for sorting of pre-DC subsets used in the in vitro differentiation assays (FIG. 3G). (A) shows pre-sorted data and (B-D) show the post-sorted re-analysis of (B) early pre-DC, (C) pre-cDC1, and (D) pre-cDC2.

FIG. 20. (A) shows expression level in terms of mean fluorescence intensity (MFI) of the side scatter area (SSC-A) indicating cellular granularity of blood pre-DC and DC subsets from five individual human donors (n=5). Error bars represent mean±SEM. (B-C) show the flow cytometry data of the relative expression of (B) CD45RA, CD169, CD11c, CD123, CD33, FcεRI, CD2, Clec9A, CD319, CD141, BTLA, CD327, CD26, CD1c, CD304 or of (C) IRF4 and IRF8 by pDC, early pre-DC, pre-cDC2, cDC2, pre-cDC1 and cDC1 defined in FIG. 3G and in FIG. 18B.

FIG. 21. shows 2D-plots showing combinations of Principal Component Analysis components 1, 2 or 3 (PC1-3) using differentially-expressed genes from the microarray analysis of FIG. 4.

FIG. 22. shows heat maps of relative expression levels of all differentially-expressed genes, with magnifications of the specific genes in early pre-DC (region within the first magnified box, middle panel) and pre-cDC1 (region within the second magnified box, middle panel) from the microarray analysis of FIG. 4.

FIG. 23. shows a Venn diagram showing genes common between the lists of cDC1 DEGs (the union of DEGs from comparing pre-cDC1 vs early pre-DC and cDC1 vs pre-cDC1) and cDC2 DEGs (the union of DEGs from comparing pre-cDC2 vs early pre-DC and cDC2 vs pre-cDC2). These 62 genes were then plotted in FIG. 4E with the logₑ fold-change values (versus early pre-DC).

FIG. 24. (A-B) show the ingenuity Pathway analysis (IPA) based on genes that were differentially-expressed between (A) cDC and early pre-DC or (B) pDC and early pre-DC. Only the DC biology-related pathways were shown, and all displayed pathways were significantly enriched (P<0.05, right-tailed Fischer's Exact Test). The heights of the bars correspond to the activation z-scores of the pathways. Enriched pathways predicted to be more activated in early pre-DC pathways and enriched pathways predicted to be more activated in cDC or pDC are shown. IPA predicts pathway activation/inhibition based on the correlation between what is known about the pathways in the literature (the Ingenuity Knowledge Base) and the directional expression observed in the user's data. IPA Upstream Regulator Analysis Whitepaper (56) and IPA Downstream Effectors Analysis Whitepaper (57) provide full description of the activation z-score calculation. (C) shows gene Ontology (GO) enrichment analysis of differentially-expressed genes (DEGs) in early pre-DC and pDC indicating biological processes that were significantly enriched (Benjamini-Hochberg adjusted p value <0.05) with genes expressed more abundantly in early pre-DC as compared to pDC. No biological process was significantly enriched with genes expressed more abundantly in pDC as compared to early pre-DC.

FIG. 25. (A) shows normalized abundance of all R mRNA in DC and pre-DC subsets obtained from the microarray analysis of FIG. 4. (B) shows polarization of naïve CD4⁺ T cells into IFNγ⁺IL-17A⁻ Th1 cells, IL-4⁺ Th2 cells, IL17A⁺IFNγ⁻ Th17 cells and IL-22⁺IFNγ⁻IL-17A⁻ Th22 cells after 6 days of culture in a mixed lymphocyte reaction with allogenic pre-DC and DC subsets (n=3). Error bars represent SEM.

FIG. 26. (A) shows the isoMAP1-2 plot of bone marrow (BM) Lin(CD3/CD7/CD14/CD15/CD19/CD34)⁻CD123^{hi} cells (upper panel) and graphics of the binned median expression of defining markers along the phenotypic progression of cells defined by the isoMAP1 dimension (lower panels). (B) shows the expression level of selected markers in the isoMAP1-2-3 3D-plots (FIG. 6C, lower left panel) corresponding to cells within the pre-DC phenograph clusters (#1 and #2) of the blood Lin⁻CD123⁺ cells isoMAP analysis. (C) shows the expression level of selected markers in the isoMAP1-2 plots (FIG. 6C, upper left panel) corresponding to cells within the pre-DC phenograph clusters (#3 and #4) of the BM Lin⁻CD123^{hi} cells isoMAP analysis. (D) shows pDC defined in BM Lin⁻CD123^{hi} (phenograph clusters #3 and #4) or blood Lin⁻CD123⁺ (phenograph cluster #7) cells of FIGS. 6A and 6B, respectively, which were exported and analyzed using the isoMAP method and subdivided into clusters using the phenograph algorithm. BM and blood concatenated and overlaid BM and blood isoMAP1/3 plots are shown (left panels). Expression level of CD2 in BM (left) and blood (right) pDC is shown in the isoMAP1/3 plot. (E) Expression level of selected markers is shown in the BM and blood concatenated isoMAP1/3 plot of FIG. 6C (right panels).

FIG. 27. is a schematic representation of the expression of major pre-DC, cDC1 and cDC2 markers as pre-DC differentiate towards cDC.

FIG. 28. is a schematic representation of the expression of major pre-DC, cDC1 and cDC2 markers as pre-DC differentiate towards cDC.

### TABLES

TABLE 1
Number of detected genes per cell in
the total DC MARS-seq experiment.

| Cell | Count |
|---|---|
| SCB_105 | 787 |
| SCB_106 | 785 |
| SCB_107 | 744 |
| SCB_108 | 774 |
| SCB_109 | 779 |
| SCB_110 | 755 |
| SCB_111 | 770 |
| SCB_112 | 740 |
| SCB_113 | 766 |
| SCB_114 | 751 |
| SCB_115 | 749 |
| SCB_116 | 780 |
| SCB_117 | 764 |
| SCB_118 | 734 |
| SCB_119 | 742 |
| SCB_120 | 787 |
| SCB_121 | 766 |
| SCB_122 | 766 |
| SCB_123 | 755 |
| SCB_124 | 758 |
| SCB_125 | 762 |
| SCB_126 | 767 |
| SCB_127 | 758 |
| SCB_128 | 756 |
| SCB_129 | 783 |
| SCB_130 | 744 |
| SCB_131 | 766 |
| SCB_132 | 729 |
| SCB_133 | 717 |
| SCB_134 | 781 |
| SCB_135 | 794 |
| SCB_136 | 775 |
| SCB_137 | 745 |
| SCB_138 | 784 |
| SCB_139 | 745 |
| SCB_140 | 748 |
| SCB_141 | 771 |
| SCB_142 | 767 |
| SCB_143 | 768 |
| SCB_144 | 670 |
| SCB_145 | 701 |
| SCB_146 | 752 |
| SCB_147 | 746 |
| SCB_148 | 726 |
| SCB_149 | 750 |
| SCB_150 | 781 |
| SCB_151 | 738 |
| SCB_152 | 775 |
| SCB_153 | 750 |
| SCB_154 | 788 |
| SCB_155 | 781 |
| SCB_156 | 773 |
| SCB_157 | 770 |
| SCB_158 | 762 |
| SCB_159 | 766 |
| SCB_160 | 768 |
| SCB_161 | 752 |
| SCB_162 | 767 |
| SCB_163 | 719 |
| SCB_164 | 748 |
| SCB_165 | 774 |
| SCB_166 | 769 |
| SCB_167 | 792 |
| SCB_168 | 772 |
| SCB_169 | 721 |
| SCB_170 | 752 |
| SCB_171 | 745 |
| SCB_172 | 749 |
| SCB_173 | 774 |
| SCB_174 | 745 |
| SCB_175 | 780 |
| SCB_176 | 763 |
| SCB_177 | 770 |
| SCB_178 | 777 |
| SCB_179 | 755 |
| SCB_180 | 719 |
| SCB_181 | 756 |
| SCB_182 | 759 |
| SCB_183 | 720 |
| SCB_184 | 730 |
| SCB_185 | 741 |
| SCB_186 | 741 |
| SCB_187 | 760 |
| SCB_188 | 783 |
| SCB_189 | 760 |
| SCB_190 | 757 |
| SCB_191 | 786 |
| SCB_192 | 753 |
| SCB_193 | 786 |
| SCB_194 | 761 |
| SCB_195 | 749 |
| SCB_196 | 737 |
| SCB_197 | 720 |
| SCB_198 | 781 |
| SCB_199 | 749 |
| SCB_200 | 780 |
| SCB_201 | 793 |
| SCB_202 | 747 |
| SCB_203 | 771 |
| SCB_204 | 719 |
| SCB_205 | 754 |
| SCB_206 | 779 |
| SCB_207 | 742 |
| SCB_208 | 750 |
| SCB_209 | 751 |
| SCB_210 | 756 |
| SCB_211 | 732 |
| SCB_212 | 760 |
| SCB_213 | 734 |
| SCB_214 | 740 |
| SCB_215 | 714 |
| SCB_216 | 727 |
| SCB_217 | 748 |
| SCB_218 | 772 |
| SCB_219 | 772 |
| SCB_220 | 743 |
| SCB_221 | 686 |
| SCB_222 | 758 |
| SCB_223 | 771 |
| SCB_224 | 766 |
| SCB_225 | 755 |
| SCB_226 | 709 |
| SCB_227 | 733 |
| SCB_228 | 758 |
| SCB_229 | 756 |
| SCB_230 | 709 |
| SCB_231 | 756 |
| SCB_232 | 748 |
| SCB_233 | 782 |
| SCB_234 | 688 |
| SCB_235 | 626 |
| SCB_236 | 730 |
| SCB_237 | 757 |
| SCB_238 | 726 |
| SCB_239 | 734 |
| SCB_240 | 757 |
| SCB_241 | 773 |
| SCB_242 | 745 |
| SCB_243 | 750 |
| SCB_244 | 725 |
| SCB_245 | 725 |
| SCB_246 | 711 |
| SCB_247 | 729 |
| SCB_248 | 722 |
| SCB_249 | 734 |
| SCB_250 | 722 |
| SCB_251 | 729 |
| SCB_252 | 725 |
| SCB_253 | 763 |
| SCB_254 | 778 |
| SCB_255 | 768 |
| SCB_256 | 748 |
| SCB_257 | 787 |
| SCB_258 | 736 |
| SCB_259 | 730 |
| SCB_260 | 782 |
| SCB_261 | 753 |
| SCB_262 | 758 |
| SCB_263 | 690 |
| SCB_264 | 735 |
| SCB_265 | 735 |
| SCB_266 | 739 |
| SCB_267 | 682 |
| SCB_268 | 788 |
| SCB_269 | 729 |
| SCB_270 | 729 |
| SCB_271 | 764 |
| SCB_272 | 746 |
| SCB_273 | 774 |
| SCB_274 | 759 |
| SCB_275 | 749 |
| SCB_276 | 773 |
| SCB_277 | 777 |
| SCB_278 | 755 |
| SCB_279 | 748 |
| SCB_280 | 755 |
| SCB_281 | 752 |
| SCB_282 | 762 |
| SCB_283 | 723 |
| SCB_284 | 742 |
| SCB_285 | 776 |
| SCB_286 | 726 |
| SCB_287 | 786 |
| SCB_1 | 721 |
| SCB_2 | 768 |
| SCB_3 | 746 |
| SCB_4 | 791 |
| SCB_5 | 734 |
| SCB_6 | 754 |
| SCB_7 | 760 |
| SCB_8 | 757 |
| SCB_9 | 763 |
| SCB_10 | 706 |
| SCB_11 | 713 |
| SCB_12 | 776 |
| SCB_13 | 749 |
| SCB_14 | 765 |
| SCB_15 | 762 |
| SCB_16 | 772 |
| SCB_17 | 767 |
| SCB_18 | 705 |
| SCB_19 | 721 |
| SCB_20 | 740 |
| SCB_21 | 765 |
| SCB_22 | 774 |
| SCB_23 | 766 |
| SCB_24 | 765 |
| SCB_25 | 682 |
| SCB_26 | 772 |
| SCB_27 | 730 |
| SCB_28 | 763 |
| SCB_29 | 735 |
| SCB_30 | 754 |
| SCB_31 | 737 |
| SCB_32 | 787 |
| SCB_33 | 758 |
| SCB_34 | 768 |
| SCB_35 | 713 |
| SCB_36 | 722 |
| SCB_37 | 765 |
| SCB_38 | 741 |
| SCB_39 | 757 |
| SCB_40 | 759 |
| SCB_41 | 750 |
| SCB_42 | 776 |
| SCB_43 | 713 |
| SCB_44 | 675 |
| SCB_45 | 775 |
| SCB_46 | 757 |
| SCB_47 | 760 |
| SCB_48 | 764 |
| SCB_49 | 730 |
| SCB_50 | 755 |
| SCB_51 | 751 |
| SCB_52 | 774 |
| SCB_53 | 743 |
| SCB_54 | 714 |
| SCB_55 | 739 |
| SCB_56 | 750 |
| SCB_57 | 758 |
| SCB_58 | 755 |
| SCB_59 | 776 |
| SCB_60 | 759 |
| SCB_61 | 697 |
| SCB_62 | 721 |
| SCB_63 | 741 |
| SCB_64 | 682 |
| SCB_65 | 756 |
| SCB_66 | 766 |
| SCB_67 | 725 |
| SCB_68 | 774 |
| SCB_69 | 733 |
| SCB_70 | 710 |
| SCB_71 | 758 |
| SCB_72 | 743 |
| SCB_73 | 758 |
| SCB_74 | 740 |
| SCB_75 | 725 |
| SCB_76 | 713 |
| SCB_77 | 735 |
| SCB_78 | 768 |
| SCB_79 | 715 |
| SCB_80 | 713 |
| SCB_81 | 751 |
| SCB_82 | 745 |
| SCB_83 | 742 |
| SCB_84 | 782 |
| SCB_85 | 783 |
| SCB_86 | 753 |
| SCB_87 | 744 |
| SCB_88 | 743 |
| SCB_89 | 741 |
| SCB_90 | 736 |
| SCB_91 | 691 |
| SCB_92 | 772 |
| SCB_93 | 764 |
| SCB_94 | 748 |
| SCB_95 | 770 |
| SCB_96 | 744 |
| SCB_97 | 732 |
| SCB_98 | 749 |
| SCB_99 | 763 |
| SCB_100 | 718 |
| SCB_101 | 781 |
| SCB_102 | 711 |
| SCB_103 | 753 |
| SCB_104 | 781 |
| SCB_360 | 761 |
| SCB_361 | 754 |
| SCB_362 | 775 |
| SCB_363 | 762 |
| SCB_364 | 779 |
| SCB_365 | 782 |
| SCB_366 | 763 |
| SCB_367 | 779 |
| SCB_368 | 786 |
| SCB_369 | 748 |
| SCB_370 | 779 |
| SCB_371 | 764 |
| SCB_372 | 745 |
| SCB_373 | 754 |
| SCB_374 | 778 |
| SCB_375 | 802 |
| SCB_376 | 788 |
| SCB_377 | 732 |
| SCB_378 | 718 |
| SCB_379 | 698 |
| SCB_380 | 761 |
| SCB_381 | 747 |
| SCB_382 | 812 |
| SCB_383 | 784 |
| SCB_384 | 781 |
| SCB_385 | 715 |
| SCB_386 | 717 |
| SCB_387 | 773 |
| SCB_388 | 699 |
| SCB_389 | 703 |
| SCB_390 | 768 |
| SCB_391 | 712 |
| SCB_392 | 759 |
| SCB_393 | 747 |
| SCB_394 | 747 |
| SCB_395 | 776 |
| SCB_396 | 794 |
| SCB_397 | 788 |
| SCB_398 | 770 |
| SCB_399 | 734 |
| SCB_400 | 719 |
| SCB_401 | 752 |
| SCB_402 | 774 |
| SCB_403 | 768 |
| SCB_404 | 754 |
| SCB_405 | 764 |
| SCB_406 | 729 |
| SCB_407 | 750 |
| SCB_408 | 731 |
| SCB_409 | 784 |
| SCB_410 | 785 |
| SCB_411 | 738 |
| SCB_412 | 775 |
| SCB_413 | 722 |
| SCB_414 | 803 |
| SCB_415 | 782 |
| SCB_416 | 778 |
| SCB_417 | 768 |
| SCB_418 | 749 |
| SCB_419 | 770 |
| SCB_420 | 731 |
| SCB_421 | 785 |
| SCB_422 | 747 |
| SCB_423 | 733 |
| SCB_424 | 732 |
| SCB_425 | 732 |
| SCB_426 | 759 |
| SCB_427 | 740 |
| SCB_428 | 741 |
| SCB_429 | 769 |
| SCB_430 | 713 |
| SCB_431 | 720 |
| SCB_432 | 773 |
| SCB_433 | 753 |
| SCB_434 | 742 |
| SCB_435 | 721 |
| SCB_436 | 798 |
| SCB_437 | 756 |
| SCB_438 | 767 |
| SCB_439 | 790 |
| SCB_440 | 768 |
| SCB_441 | 771 |
| SCB_442 | 738 |
| SCB_443 | 760 |
| SCB_444 | 765 |
| SCB_445 | 770 |
| SCB_446 | 752 |
| SCB_447 | 799 |
| SCB_448 | 749 |
| SCB_449 | 712 |
| SCB_450 | 777 |
| SCB_451 | 700 |
| SCB_452 | 748 |
| SCB_453 | 795 |
| SCB_454 | 738 |
| SCB_455 | 782 |
| SCB_456 | 742 |
| SCB_457 | 763 |
| SCB_458 | 762 |
| SCB_459 | 665 |
| SCB_460 | 707 |
| SCB_511 | 787 |
| SCB_512 | 779 |
| SCB_513 | 753 |
| SCB_514 | 766 |
| SCB_515 | 775 |
| SCB_516 | 771 |
| SCB_517 | 777 |
| SCB_518 | 774 |
| SCB_519 | 757 |
| SCB_520 | 756 |
| SCB_521 | 750 |
| SCB_522 | 758 |
| SCB_523 | 719 |
| SCB_524 | 731 |
| SCB_525 | 736 |
| SCB_526 | 744 |
| SCB_527 | 765 |
| SCB_528 | 755 |
| SCB_529 | 737 |
| SCB_530 | 768 |
| SCB_531 | 769 |
| SCB_532 | 796 |
| SCB_533 | 757 |
| SCB_534 | 726 |
| SCB_535 | 741 |
| SCB_536 | 731 |
| SCB_537 | 802 |
| SCB_538 | 731 |
| SCB_539 | 715 |
| SCB_540 | 785 |
| SCB_541 | 758 |
| SCB_542 | 779 |
| SCB_543 | 800 |
| SCB_544 | 741 |
| SCB_545 | 779 |
| SCB_546 | 729 |
| SCB_547 | 737 |
| SCB_548 | 773 |
| SCB_549 | 787 |
| SCB_550 | 771 |
| SCB_551 | 750 |
| SCB_552 | 746 |
| SCB_553 | 742 |
| SCB_554 | 767 |
| SCB_555 | 743 |
| SCB_556 | 750 |
| SCB_557 | 744 |
| SCB_558 | 756 |
| SCB_559 | 765 |
| SCB_560 | 759 |
| SCB_561 | 741 |
| SCB_562 | 730 |
| SCB_563 | 762 |
| SCB_564 | 737 |
| SCB_565 | 770 |
| SCB_566 | 774 |
| SCB_567 | 720 |
| SCB_568 | 763 |
| SCB_569 | 725 |
| SCB_570 | 735 |
| SCB_571 | 713 |
| SCB_572 | 747 |
| SCB_573 | 750 |
| SCB_574 | 763 |
| SCB_575 | 768 |
| SCB_576 | 800 |
| SCB_577 | 788 |
| SCB_578 | 726 |
| SCB_579 | 761 |
| SCB_580 | 764 |
| SCB_581 | 735 |
| SCB_582 | 729 |
| SCB_583 | 812 |
| SCB_584 | 718 |
| SCB_585 | 745 |
| SCB_586 | 742 |
| SCB_587 | 728 |
| SCB_588 | 752 |
| SCB_589 | 758 |
| SCB_590 | 769 |
| SCB_591 | 742 |
| SCB_592 | 752 |
| SCB_593 | 777 |
| SCB_594 | 718 |
| SCB_595 | 777 |
| SCB_596 | 776 |
| SCB_597 | 706 |
| SCB_598 | 750 |
| SCB_599 | 777 |
| SCB_600 | 761 |
| SCB_601 | 731 |
| SCB_602 | 729 |
| SCB_603 | 776 |
| SCB_604 | 717 |
| SCB_605 | 747 |
| SCB_606 | 757 |
| SCB_607 | 737 |
| SCB_608 | 760 |
| SCB_609 | 804 |
| SCB_610 | 758 |
| SCB_611 | 771 |
| SCB_612 | 767 |
| SCB_613 | 762 |
| SCB_614 | 747 |
| SCB_615 | 764 |
| SCB_616 | 761 |
| SCB_617 | 746 |
| SCB_618 | 782 |
| SCB_619 | 777 |
| SCB_620 | 700 |
| SCB_621 | 757 |
| SCB_622 | 747 |
| SCB_623 | 770 |
| SCB_624 | 772 |
| SCB_625 | 792 |
| SCB_626 | 733 |
| SCB_627 | 776 |
| SCB_699 | 769 |
| SCB_700 | 805 |
| SCB_701 | 799 |
| SCB_702 | 712 |
| SCB_703 | 672 |
| SCB_704 | 788 |
| SCB_705 | 672 |
| SCB_706 | 755 |
| SCB_707 | 708 |
| SCB_708 | 709 |
| SCB_709 | 752 |
| SCB_710 | 718 |
| SCB_288 | 716 |
| SCB_289 | 767 |
| SCB_290 | 770 |
| SCB_291 | 720 |
| SCB_292 | 704 |
| SCB_293 | 787 |
| SCB_294 | 732 |
| SCB_295 | 728 |
| SCB_296 | 746 |
| SCB_297 | 782 |
| SCB_298 | 682 |
| SCB_299 | 760 |
| SCB_300 | 687 |
| SCB_301 | 745 |
| SCB_302 | 777 |
| SCB_303 | 701 |
| SCB_304 | 773 |
| SCB_305 | 748 |
| SCB_306 | 772 |
| SCB_307 | 795 |
| SCB_308 | 753 |
| SCB_309 | 753 |
| SCB_310 | 714 |
| SCB_311 | 758 |
| SCB_312 | 695 |
| SCB_313 | 748 |
| SCB_314 | 747 |
| SCB_315 | 750 |
| SCB_316 | 746 |
| SCB_317 | 774 |
| SCB_318 | 723 |
| SCB_319 | 753 |
| SCB_320 | 741 |
| SCB_321 | 718 |
| SCB_322 | 744 |
| SCB_323 | 750 |
| SCB_324 | 711 |
| SCB_325 | 731 |
| SCB_326 | 764 |
| SCB_327 | 699 |
| SCB_328 | 755 |
| SCB_329 | 716 |
| SCB_330 | 783 |
| SCB_331 | 739 |
| SCB_332 | 747 |
| SCB_333 | 752 |
| SCB_334 | 766 |
| SCB_335 | 715 |
| SCB_336 | 765 |
| SCB_337 | 745 |
| SCB_338 | 698 |
| SCB_339 | 770 |
| SCB_340 | 730 |
| SCB_341 | 767 |
| SCB_342 | 786 |
| SCB_343 | 709 |
| SCB_344 | 767 |
| SCB_345 | 778 |
| SCB_346 | 745 |
| SCB_347 | 778 |
| SCB_348 | 759 |
| SCB_349 | 755 |
| SCB_350 | 733 |
| SCB_351 | 759 |
| SCB_352 | 708 |
| SCB_353 | 721 |
| SCB_354 | 792 |
| SCB_355 | 761 |
| SCB_356 | 686 |
| SCB_357 | 733 |
| SCB_358 | 765 |
| SCB_359 | 756 |
| SCB_628 | 763 |
| SCB_629 | 715 |
| SCB_630 | 719 |
| SCB_631 | 774 |
| SCB_632 | 691 |
| SCB_633 | 691 |
| SCB_634 | 687 |
| SCB_635 | 706 |
| SCB_636 | 708 |
| SCB_637 | 702 |
| SCB_638 | 743 |
| SCB_639 | 752 |
| SCB_640 | 772 |
| SCB_641 | 739 |
| SCB_642 | 733 |
| SCB_643 | 767 |
| SCB_644 | 735 |
| SCB_645 | 756 |
| SCB_646 | 775 |
| SCB_647 | 728 |
| SCB_648 | 750 |
| SCB_649 | 768 |
| SCB_461 | 723 |
| SCB_462 | 804 |
| SCB_463 | 713 |
| SCB_464 | 699 |
| SCB_465 | 766 |
| SCB_466 | 768 |
| SCB_467 | 759 |
| SCB_468 | 765 |
| SCB_469 | 784 |
| SCB_470 | 702 |
| SCB_471 | 703 |
| SCB_472 | 775 |
| SCB_473 | 753 |
| SCB_474 | 764 |
| SCB_475 | 680 |
| SCB_476 | 768 |
| SCB_477 | 709 |
| SCB_478 | 761 |
| SCB_479 | 777 |
| SCB_480 | 719 |
| SCB_481 | 761 |
| SCB_482 | 784 |
| SCB_483 | 718 |
| SCB_484 | 771 |
| SCB_485 | 766 |
| SCB_486 | 733 |
| SCB_487 | 767 |
| SCB_488 | 793 |
| SCB_489 | 758 |
| SCB_490 | 768 |
| SCB_491 | 764 |
| SCB_492 | 811 |
| SCB_493 | 779 |
| SCB_494 | 691 |
| SCB_495 | 694 |
| SCB_496 | 766 |
| SCB_497 | 756 |
| SCB_498 | 780 |
| SCB_499 | 770 |
| SCB_500 | 757 |
| SCB_501 | 776 |
| SCB_502 | 806 |
| SCB_503 | 737 |
| SCB_504 | 769 |
| SCB_505 | 754 |
| SCB_506 | 736 |
| SCB_507 | 773 |
| SCB_508 | 726 |
| SCB_509 | 773 |
| SCB_510 | 756 |
| SCB_677 | 690 |
| SCB_678 | 728 |
| SCB_679 | 725 |
| SCB_680 | 749 |
| SCB_681 | 759 |
| SCB_682 | 746 |
| SCB_683 | 740 |
| SCB_684 | 689 |
| SCB_685 | 698 |
| SCB_686 | 737 |
| SCB_687 | 741 |
| SCB_688 | 729 |
| SCB_689 | 808 |
| SCB_690 | 701 |
| SCB_691 | 789 |
| SCB_692 | 775 |
| SCB_693 | 811 |
| SCB_694 | 727 |
| SCB_695 | 778 |
| SCB_696 | 718 |
| SCB_697 | 724 |
| SCB_698 | 690 |
| SCB_650 | 797 |
| SCB_651 | 736 |
| SCB_652 | 773 |
| SCB_653 | 703 |
| SCB_654 | 772 |
| SCB_655 | 769 |
| SCB_656 | 797 |
| SCB_657 | 765 |
| SCB_658 | 764 |
| SCB_659 | 741 |
| SCB_660 | 732 |
| SCB_661 | 768 |
| SCB_662 | 758 |
| SCB_663 | 773 |
| SCB_664 | 753 |
| SCB_665 | 745 |
| SCB_666 | 709 |
| SCB_667 | 705 |
| SCB_668 | 662 |
| SCB_669 | 729 |
| SCB_670 | 784 |
| SCB_671 | 726 |
| SCB_672 | 691 |
| SCB_673 | 782 |
| SCB_674 | 651 |
| SCB_675 | 760 |
| SCB_676 | 705 |

TABLE 2
DC subsets signature genes derived from Gene Expression Omnibus data
series GSE35457 and used for MARS-seq and C1 data analyses.

| | | | | cDC2 signature |
|---|---|---|---|---|
| pDC signature genes | | cDC1 signature genes | | genes |
| ABCA7 | MTMR2 | ABCB4 | STX11 | ABCG1 |
| ABCB6 | MUPCDH | ABI3 | STX6 | ACP5 |
| ABHD15 | MX1 | ABR | SVIL | ACP6 |
| ABTB2 | MYB | ACER3 | SWAP70 | ACSL1 |
| ACACB | MYBPH | ACOT11 | SYN1 | ACSL5 |
| ACN9 | MYH3 | ACPP | SYT11 | ACSS2 |
| ACSBG1 | MYL6B | ACTA2 | SYTL3 | ACTB |
| ACSM3 | N4BP2L1 | ACVRL1 | TBL1X | ACTR3 |
| ADA | N6AMT1 | ADAM15 | TBXAS1 | ADAD2 |
| ADAM19 | NADK | ADAM8 | TESC | ADAM28 |
| ADARB1 | NAT8L | ADAMTSL4 | TICAM2 | ADORA2B |
| ADAT3 | NCF1C | ADAP1 | TIMP1 | ADORA3 |
| ADC | NCLN | AGTPBP1 | TIPARP | AGPAT1 |
| ADI1 | NCRNA00153 | ALDH3B1 | TKT | AGPS |
| AEBP1 | NDST2 | ALOX5 | TLE4 | AIG1 |
| AHI1 | NEK8 | AMICA1 | TLR2 | AIM2 |
| AJAPI | NFATC2IP | AMOT | TLR5 | ALDH1A1 |
| AKR1C3 | NFX1 | ANG | TLR8 | ALDH3A2 |
| ALDH5A1 | NGLY1 | ANXA1 | TM6SF1 | AMY1A |
| ALOX5AP | NHEDC1 | ANXA2 | TMC6 | ANPEP |
| ANKRD33 | NIN | ANXA2P1 | TMEM154 | ANXA6 |
| APOBEC3D | NIPA1 | ANXA5 | TMEM173 | AP3M2 |
| APP | NLRP2 | AOAH | TMEM2 | APOL1 |
| ARHGAP25 | NLRP7 | APAF1 | TMEM71 | APOL2 |
| ARHGAP27 | NOP56 | APLP2 | TNFAIP2 | APOL3 |
| ARHGAP9 | NOTCH3 | ARAP3 | TNFRSF10D | ASAP1 |
| ARHGEF10 | NOTCH4 | ARHGAP10 | TNFRSF1A | ASB2 |
| ARHGEF4 | NPAL3 | ARL4A | TNFRSF1B | ATG3 |
| ARID3A | NPCI | ARRDC2 | TNFSF10 | ATL1 |
| ARMC5 | NPC2 | ASCL2 | TNFSF12 | ATP1A1 |
| ARMET | NR5Al | ASGR1 | TNFSF13B | AZI1 |
| ARRDC5 | NRP1 | ASGR2 | TOB1 | B4GALT5 |
| ASIP | NTAN1 | ATP1B1 | TPPP3 | BAG3 |
| ATP10A | NUCB2 | ATP6V1B2 | TREM1 | BATF3 |
| ATP13A2 | NUMA1 | BACH2 | TRIB1 | BCAR3 |
| ATP2A3 | OAS1 | BATF | TRIB2 | BCL6 |
| ATP8B2 | ODC1 | BLVRA | TSC22D3 | BEND5 |
| AUTS2 | OFD1 | BTBD11 | TSPAN32 | BIK |
| AVEN | OGT | C10orf11 | TSPAN4 | BIVM |
| B4GALT1 | OPN3 | C10orf54 | TSPO | BTLA |
| BAIAP2L1 | OPTN | C15orf39 | TTYH3 | C10orf105 |
| BCAS4 | OR3A3 | C16orf7 | UBAC1 | C10orf64 |
| BCL11A | P2RX1 | C17orf44 | UPP1 | Cl3orf15 |
| BEND6 | P4HB | C2CD2 | USP3 | C13orf31 |
| BLK | PACAP | C3orf59 | VCAN | C15orf38 |
| BLNK | PACSIN1 | C4orf18 | VENTX | C17orf58 |
| BSPRY | PAFAH2 | C9orf72 | VIPR1 | C1orf115 |
| BTAF1 | PAG1 | CA2 | VPS37C | C1orf162 |
| BTG1 | PANX2 | CACNA2D3 | VSIG4 | C1orf165 |
| C10orf141 | PAPLN | CALHM2 | XAF1 | C1orf186 |
| C10orf47 | PARP10 | CAPN2 | XYLT1 | Clorf21 |
| C10orf58 | PARVB | CARD16 | YIF1B | C1orf24 |
| C11orf24 | PBX3 | CARD9 | ZAK | Clorf51 |
| C11orf67 | PCNT | CASP1 | ZBP1 | C1orf54 |
| Cl1orf80 | PCNX | CASP4 | ZEB2 | C20orf27 |
| C12orf23 | PCSK4 | CAST | ZFAND5 | C21orf63 |
| C12orf44 | PDCD4 | CCL5 | ZFP36 | C5orf30 |
| C12orf57 | PDIA4 | CCND2 | ZNF562 | C8orf47 |
| C13orf18 | PDXP | CCR6 | ZYG11B | CADM1 |
| C14orf4 | PFKFB2 | CD14 | | CAMK2D |
| Cl4orf45 | PFKP | CD151 | | CAMP |
| C16orf33 | PGD | CD163 | | CBL |
| C16orf58 | PGM2L1 | CD1A | | CCDC6 |
| Cl6orf93 | PHACTR1 | CD1B | | CCDC62 |
| C18orf25 | PHEX | CD1C | | CCDC90A |
| C18orf8 | PHF16 | CD1D | | CCND1 |
| C1orf109 | PI4KAP2 | CD1E | | CCR9 |
| C20orf100 | PIK3AP1 | CD2 | | CD226 |
| C20orf103 | PIK3CD | CD209 | | CD38 |
| C20orf132 | PIK4CA | CD244 | | CD48 |
| C21orf2 | PLAC8 | CD300A | | CD59 |
| C2orf55 | PLAU | CD300C | | CDCA7 |
| C3orf21 | PLD6 | CD300LF | | CDH2 |
| C4BPB | PLEKHG4 | CD33 | | CDK2AP1 |
| C5orf62 | PLP2 | CD5 | | CDK6 |
| C6orf170 | PLS3 | CD52 | | CHD7 |
| C7orf41 | PMEPA1 | CD69 | | CHST2 |
| C7orf54 | PNOC | CDC42EP4 | | CLEC1A |
| C8orf13 | POLB | CDCP1 | | CLEC9A |
| C9orf127 | POLE | CDH23 | | CLNK |
| C9orf128 | POMGNT1 | CDS1 | | CLSTN2 |
| C9orf142 | POU4F1 | CEBPA | | CNTLN |
| C9orf37 | PPM1J | CEBPB | | CPNE3 |
| C9orf45 | PPP1R14A | CEBPD | | CREG1 |
| C9orf91 | PPP1R14B | CENPN | | CSRP1 |
| C9orf95 | PPP1R16B | CENTA1 | | CST3 |
| CA8 | PPP2R1B | CENTG3 | | CTPS2 |
| CADM4 | PPP2R5C | CES1 | | CXCL16 |
| CARD11 | PRAGMIN | CFD | | CXCL9 |
| CASZ1 | PRIM1 | CFP | | CYB5R3 |
| CBLB | PRKCB | CHD1 | | CYP2E1 |
| CBX4 | PRKCB1 | CHST13 | | DBN1 |
| CBX6 | PRKD1 | CIDEB | | DCLRE1A |
| CCDC102A | PROC | CLEC10A | | DCTPP1 |
| CCDC50 | PSCD4 | CLEC12A | | DFNA5 |
| CCDC69 | PSD4 | CLEC4A | | DHCR24 |
| CCR2 | PTCRA | CLEC4F | | DHRS3 |
| CCR3 | PTGDS | CMTM1 | | DLG3 |
| CCR7 | PTGR1 | COL9A2 | | DOCK7 |
| CCS | PTK7 | COQ10A | | DPP4 |
| CD164 | PTPRCAP | CPNE8 | | DSE |
| CD247 | PTPRM | CPPED1 | | DYSF |
| CD2AP | PVRL1 | CREB5 | | EGLN3 |
| CD320 | QDPR | CRTAP | | EHD4 |
| CD36 | RAB15 | CRYL1 | | ELOVL5 |
| CD4 | RAB38 | CRYZL1 | | ENOX1 |
| CD68 | RAB40B | CSF1R | | ENPP1 |
| CD7 | RAB9P1 | CSF3R | | ENPP3 |
| CD99 | RABGAP1L | CST7 | | ENPP4 |
| CDC14A | RALGPS1 | CSTA | | ERAP2 |
| CDCA7L | RASD1 | CTSH | | ERMP1 |
| CDH1 | RBM38 | CX3CR1 | | ERO1L |
| CDK2 | RECQL5 | CXCR7 | | EV12A |
| CDK5R1 | RELN | CYBRD1 | | EVL |
| CDKN2D | REX02 | CYFIP1 | | FAH |
| CDR2 | RGS1 | DAGLB | | FAM102A |
| CDYL | RGS7 | DDX60L | | FAM125B |
| CENPV | RHBDF2 | DEM1 | | FAM129A |
| CETP | RIMS3 | DENND3 | | FAM149A |
| CHST12 | RLTPR | DEPDC6 | | FAMI60A2 |
| CHST15 | RNASE6 | DHRS9 | | FAM20C |
| C1B2 | RNASEL | DOK2 | | FAM57A |
| CIRBP | RNF11 | DPEP2 | | FAR2 |
| CLDN23 | RNF121 | DPYD | | FARS2 |
| CLEC4C | RNF165 | DTD1 | | FBXL20 |
| CLIC3 | RPA1 | DTNA | | FKBP1B |
| CLN8 | RPP25 | ECGF1 | | FLJ10916 |
| CMTM3 | RPPH1 | EFNB1 | | FLJ22795 |
| CNTNAP1 | RPS6KA2 | EMP1 | | FLT3 |
| COBL | RPS6KA4 | EMR2 | | FMNL2 |
| COBLL1 | RRBP1 | EMR3 | | FNBP1 |
| COL24A1 | RSPH1 | ENHO | | FN1P2 |
| CORO1C | RTKN | ENTPD1 | | FUCA1 |
| CPLX1 | RUNX2 | EPB41L2 | | FUT8 |
| CREB3L2 | RWDD2A | EPB41L3 | | GCET2 |
| CRTC3 | SAP130 | EPSTI1 | | GFOD1 |
| CRYM | SBDS | ERMAP | | GINS2 |
| CTNS | SBF1 | ETS2 | | GLTP |
| CTSB | SCAMPS | FI3A1 | | GNAZ |
| CTSC | SCARA5 | FAM102B | | GPER |
| CTSL2 | SCARB1 | FAM104B | | GPR126 |
| CUEDC1 | SCARB2 | FAM109A | | GPRIN3 |
| CUTL1 | SCN9A | FAM1I10A | | GPSM1 |
| CUX2 | SCYL3 | FAM111A | | GPT2 |
| CXCR3 | SDC1 | FAM129B | | GSTP1 |
| CXorf12 | SDK2 | FAM38A | | GYPC |
| CXorf57 | SEC11C | FAM46A | | H2AFY2 |
| CXXC5 | SEC61A1 | FBLN2 | | HCP5 |
| CYBASC3 | SEC61A2 | FBN2 | | HLA-DOB |
| CYBB | SEC61B | FCGBP | | HLA-DPA1 |
| CYFIP2 | SEL1L3 | FCGR2A | | HLA-DPB1 |
| CYP2J2 | SELL | FCGR2B | | HLA-DQB1 |
| CYP46A1 | SELS | FCN1 | | HLA-DRB1 |
| CYSLTR1 | SEMA4D | FCRLB | | HLA-DRB3 |
| CYTH4 | SEMA5A | FGD4 | | HLA-DRB4 |
| CYYR1 | SEPHS1 | FILIP1L | | HMOX1 |
| DAAM1 | SERPINF1 | FLVCR2 | | HN1 |
| DAB2 | SERPING1 | FOSB | | HOXA9 |
| DACH1 | SETBP1 | FOXO1 | | HPS5 |
| DAPK2 | SH2D3C | FPR1 | | HSD17B8 |
| DBNDDI | SH3D 19 | FPR3 | | HSDL2 |
| DCK | SH3PXD2A | FRAT2 | | HYAL3 |
| DCPS | SHD | FXYD5 | | ICA1 |
| DDB1 | SIDT1 | FYB | | ICAM3 |
| DDIT4 | SIK1 | GABBR1 | | ID2 |
| DEDD2 | SIRPB1 | GADD45B | | IDO1 |
| DERL3 | SIVA | GALM | | IDO2 |
| DEXI | SIVA1 | GAPDH | | IFNGR2 |
| DHRS7 | SLA2 | GBP1 | | IFT20 |
| DHTKDI | SLA2MF6 | GBP2 | | IL15 |
| DIP2A | SLC15A4 | GBP3 | | INADL |
| DKFZP58611420 | SLC20A1 | GBP4 | | INDO |
| DKFZp761P0423 | SLC23A1 | GBP5 | | IRAK2 |
| DNASE2 | SLC25A4 | GHRL | | ITGB7 |
| DPPA4 | SLC29A1 | GIMAP1 | | ITPR3 |
| DRD4 | SLC2A1 | GIMAP2 | | KATNA1 |
| DSG2 | SLC2A6 | GIMAP4 | | KIAA1598 |
| DSN1 | SLC2A8 | GIMAP6 | | KIAA1688 |
| DTX2 | SLC35A3 | GIMAP7 | | KIFI6B |
| DUSP28 | SLC35C2 | GIMAP8 | | KIF20B |
| DUSP5 | SLC35F3 | GK | | KIT |
| DYRK4 | SLC37A1 | GLIPR2 | | KLHL22 |
| E2F2 | SLC39A6 | GPBARI | | KLHL5 |
| E2F5 | SLC3A2 | GPRI62 | | KLRG1 |
| EBI2 | SLC43A3 | GPR44 | | LAT |
| EIF4A3 | SLC44A2 | GRK5 | | LFNG |
| EIF4ENIF1 | SLC47A1 | HBEGF | | LIMAl |
| ELMO2 | SLC7A5 | HDAC4 | | LMNA |
| EMID2 | SLC7A6 | HK1 | | LOC100133583 |
| ENOSFI | SLC9A3R1 | HK2 | | LOC100133866 |
| ENPP2 | SLFN11 | HK3 | | LOC150223 |
| EPDR1 | SLITRK5 | HNMT | | LOC25845 |
| EPHB1 | SMARCAL1 | HSPA1A | | LOC439949 |
| ERCC1 | SMC6 | HSPA6 | | LOC642073 |
| ERN1 | SMPD3 | HSPA7 | | LOC642590 |
| ESR2 | SNAP91 | ICAM2 | | LOC645638 |
| ETS1 | SNCA | IER5 | | LOC649143 |
| FAM107B | SNRP25 | IFI30 | | LOC653344 |
| FAM108C1 | SNRPN | IFI6 | | L00730101 |
| FAM113B | SORL1 | IFIH1 | | LONRF1 |
| FAM129C | SPCS1 | IFIT1 | | LPAR5 |
| FAM167A | SPHK1 | IFIT3 | | LPCAT2 |
| FAM43A | SPIB | IFITM1 | | LRBA |
| FAM65A | SPNS3 | IFITM2 | | LRRC1 |
| FAM81A | SPOCK2 | IFITM3 | | LRRCC1 |
| FAM82A2 | SRPR | IFT57 | | LRRK2 |
| FANCD2 | SRPX | IGLL1 | | LYRM4 |
| 1413X018 | SSR4 | IGSF6 | | MARCKSL1 |
| FCHSD2 | ST3GAL2 | IL13RA1 | | MATK |
| FCRLA | ST3GAL4 | IL17RA | | MCM4 |
| FEZ2 | ST6GALNAC4 | IL1B | | MESP1 |
| FGFR3 | ST6GALNAC6 | IL1R1 | | MFNG |
| FHL1 | STAG3L2 | IL1R2 | | MGC4677 |
| FLJ21986 | STAG3L3 | IL1RN | | MIST |
| FLJ42627 | STAMBPL1 | INPP1 | | MMP25 |
| FMNL3 | STAT4 | IRAK3 | | MND1 |
| FYCO1 | STK11IP | IRF1I | | MPP3 |
| FZD3 | STK32B | ISG15 | | MYC |
| GAL3ST4 | STMN1 | ITGA5 | | MYLK |
| GARNL4 | STOX1 | ITGAM | | MYO1D |
| GAS6 | STT3A | ITSNI | | NAAA |
| GF11 | SUGT1 | JDP2 | | NAALADL1 |
| GGA2 | SUPT3H | JHDM1D | | NAP1L1 |
| GGH | SUPT5H | JUN | | NAV1 |
| GINS3 | SUSD1 | JUP | | NBEAL2 |
| GLCE | SYCP2L | KCNK13 | | NCALD |
| GLDN | SYS1 | KCNQ1 | | NCKAP5 |
| GLS | SYTL2 | KIAA0922 | | NET1 |
| GLT25D1 | TACC1 | KIAA1683 | | NET02 |
| GLT8D1 | TARBP1 | KLF11 | | NLRX1 |
| GNG7 | TATDN3 | KLF2 | | NMNAT3 |
| GPM6B | TAX1BP3 | KLF4 | | OSBPL3 |
| GPR114 | TBCID14 | KLF9 | | OSBPL9 |
| GPR183 | TBC1D16 | LACTB | | P2RY10 |
| GPRC5C | TBC1D4 | LAMP3 | | PAM |
| GPX7 | TBX19 | LAYN | | PAPSS1 |
| GRAMD1B | TCF3 | LDLR | | PARM1 |
| GR14 | TCF4 | LGALS1 | | PARP3 |
| GRIN1 | TCL1A | LILRA2 | | PDE8B |
| GSDMB | TCL1B | LILRA3 | | PDLIM7 |
| GZMB | TEX2 | LILRA6 | | PFKFB3 |
| GZMH | TFTP11 | LILRB3 | | PIGZ |
| HCST | TGFBR2 | LIMCH1 | | PIK3CB |
| HERC5 | TLCD1 | LIMS1 | | PITPNC1 |
| HERPUD1 | TLR7 | LMO2 | | PITPNM1 |
| HHAT | TLR9 | LOC100129550 | | PKP2 |
| HIGD1A | TM7SF2 | LOC100130520 | | PKP4 |
| HIST1H213D | TM9SF2 | LOC100170939 | | PLCD1 |
| HIST1H2BK | TMEM109 | LOC143941 | | PLEKHA5 |
| HOXB2 | TMEM141 | LOC153561 | | PLEKHA6 |
| HPS4 | TMEM149 | LOC338758 | | PLEKHO2 |
| HRASLS | T EM17013 | LOC391075 | | PLXNA1 |
| HSP90B1 | TMEM175 | L00644237 | | PLXNB1 |
| ITVCNI | TMEM187 | L00645626 | | PMM1 |
| IDH3A | TMEM194A | L00648984 | | PNLDC1 |
| 1F144 | TMEM194B | L00653778 | | PNMA1 |
| IF144L | TMEM44 | LOC654103 | | POLA2 |
| 1F1T2 | TMEM53 | LOC728093 | | PPA1 |
| IFNAR1 | TMEM63A | LOC728519 | | PPAP2A |
| IFNAR2 | TMEM91 | LOC728666 | | PPM1H |
| IGF2R | TMEM98 | LOC728855 | | PPM1M |
| IGFBP3 | TNFRSF17 | LOC729708 | | PPT1 |
| IGJ | TNFRSF21 | LOC730994 | | PPY |
| LI8RAP | TNNI2 | LOC731486 | | PRKCZ |
| IL28RA | TOM1 | LOC88523 | | PSEN2 |
| IL3RA | TOX2 | LRRC25 | | PSMB9 |
| INSM1 | TP53113 | LRRC33 | | PTGER2 |
| INTS12 | TPM2 | LST1 | | PTK2 |
| IRF4 | TPRG1L | LYL1 | | PTPLB |
| IRF7 | TPST2 | LYST | | QPRT |
| ISCU | TRAF3 | MAFB | | RAB11FIP4 |
| ITCH | TRO | MAP3K6 | | RAB30 |
| ITGAE | TRPM2 | MARCO | | RAB32 |
| ITM2C | TSEN54 | MBOAT7 | | RAB33A |
| KANK1 | TSPAN13 | MEFV | | RAB3IP |
| KATNAL1 | TSPAN3 | MEGF9 | | RAB7B |
| KCNA5 | TSPYL2 | MLKL | | RAB7L1 |
| KCNH8 | TUBB6 | MMD | | RAB8B |
| KCNK1 | TUBG1 | MOV10 | | RALB |
| KCNK10 | TUBG2 | MPZL2 | | RASGRP3 |
| KCNK17 | TULP4 | MS4A14 | | RGS10 |
| KCTD5 | TXN | MS4A7 | | RGS12 |
| KIAA0226 | TXNDC3 | MSLN | | RUSC1 |
| KIAA0513 | TXNDC5 | MSN | | RYK |
| KIAA1147 | UBE2E3 | MT1A | | S100A10 |
| KIAA1274 | UBE2J1 | MTMR11 | | Septin 3 |
| KIAAI370 | UBQLNL | MYBPC3 | | SERP1NB6 |
| KIAA1545 | UGCG | MYO1A | | SERPINF2 |
| KIAAI641 | ULK1 | MYO1F | | SH3RF2 |
| KIAA1984 | UNC93B1 | MYO5C | | SHE |
| KIF13B | USF2 | MYPOP | | SIGLEC10 |
| KIF26B | USP11 | NACC2 | | SIGLECP3 |
| KLHLI3 | USP24 | NCH | | SLA |
| KLHL3 | USP36 | NFE2 | | SLAMF7 |
| KMO | VASH2 | NINJ2 | | SLAMF8 |
| KRT5 | VEGFB | NLRP12 | | SLC1A3 |
| L3MBTL3 | VEZF1 | NLRP3 | | SLC24A4 |
| LA1R1 | VIPR2 | NOD2 | | SLC25A25 |
| LAMCI | WDR19 | NR1H3 | | SLC39A8 |
| LAMP1 | WDR51A | NR4A2 | | SLC44A1 |
| LAMP2 | WNT10A | OAF | | SLC46A3 |
| LAPTM4B | XBP1 | OAS3 | | SLC9A9 |
| LASS6 | YPEL1 | OLFM1 | | SLCO3A1 |
| LBH | ZC3H5 | OSCAR | | SMO |
| LDOCI | ZCCHC11 | P2RY13 | | SNORA57 |
| LEPREL1 | ZCWPW1 | P2RY2 | | SNX22 |
| LGMN | ZDHHC14 | P2RY5 | | SNX3 |
| LRFPL2 | ZDHHC17 | PAPSS2 | | SNX30 |
| LILRA4 | ZDHHC23 | PARP14 | | SP140 |
| LILRB4 | ZDHHC4 | PARP9 | | SPATS2L |
| LIME1 | ZDHHC8 | PCCA | | SPI1 |
| LMNB2 | ZDHHC9 | PCK2 | | SPIN3 |
| LOC100128410 | ZFYVE26 | PCSK5 | | SPNS1 |
| LOC100129466 | ZHX2 | PEAI5 | | SPRY2 |
| LOC100129673 | ZKSCAN4 | PFKFB4 | | ST3GAL5 |
| LOC100130633 | ZMYM6 | PHCA | | ST5 |
| LOC100131289 | ZMYND11 | PID1 | | ST6GALNAC2 |
| LOC100132299 | ZNF175 | PILRA | | ST7 |
| LOC100132740 | ZNF185 | PION | | STK39 |
| LOC100134134 | ZNF2I9 | PIP3-E | | STOM |
| LOC100190939 | ZNF521 | PIP4K2A | | STX3 |
| LOC132241 | ZNF556 | NUB | | SUOX |
| LOC201175 | ZNF589 | PLA2G7 | | SUSD3 |
| LOC221442 | ZNF706 | PLSCR3 | | TACSTD2 |
| LOC283874 | ZNF767 | PLXDC2 | | TANC2 |
| LOC285296 | ZNF789 | PNPLA6 | | TAP1 |
| LOC285359 | ZSCAN16 | PPEF1 | | TAP2 |
| LOC347544 | | PPFIA4 | | TCEA3 |
| LOC387841 | | PPFIBP2 | | TCEAL3 |
| LOC387882 | | PPM1F | | TGM2 |
| LOC389442 | | PQLC3 | | THBD |
| LOC389816 | | PRAM1 | | THEM4 |
| LOC399804 | | PRDM1 | | TJP2 |
| LOC400027 | | PRIC2S | | TLR10 |
| LOC400657 | | PRKCD | | TL23 |
| LOC442535 | | PSRC1 | | TMEM106C |
| LOC550112 | | PSTPIP2 | | TMEM14A |
| LOC641298 | | PTAFR | | TMEM97 |
| LOC642031 | | PTGER4 | | TOX |
| LOC642299 | | PTGS1 | | TPMT |
| LOC642755 | | PTGS2 | | TRAF3IP2 |
| LOC643384 | | PTK6 | | TRAF5 |
| LOC644879 | | PTPN12 | | TRIB3 |
| LOC646576 | | PYGL | | TSHZ3 |
| LOC647000 | | RAB24 | | TSPAN2 |
| LOC647886 | | RAB27A | | TSPAN33 |
| LOC650114 | | RARA | | TSPYL3 |
| LOC651957 | | RARRES3 | | TTF2 |
| LOC652128 | | RASSF4 | | TUBA4A |
| LOC653158 | | RCBTB2 | | VAC14 |
| LOC728308 | | RGL1 | | VAV3 |
| LOC728661 | | RHOU | | VCAM1 |
| LOC728715 | | RIN2 | | VPS37D |
| LOC728743 | | RIPK5 | | WARS |
| LOC729148 | | RNASE2 | | WDFY4 |
| LOC729406 | | RPGRIP1 | | WDR41 |
| LOC729764 | | RTN1 | | WDR91 |
| LOC9143I | | RXRA | | YEATS2 |
| LOXL4 | | S100Al2 | | ZBTB46 |
| LPXN | | S100A4 | | ZDHHC18 |
| LRP5 | | S100A8 | | ZFP36L1 |
| LRP8 | | S100A9 | | ZMYND15 |
| LRRC26 | | SAMD9L | | ZNF232 |
| LRRC36 | | SAP30 | | ZNF366 |
| LSS | | SCO2 | | ZNF532 |
| LTB | | SCPEP1 | | ZNF627 |
| LTK | | SDHALP1 | | ZNF662 |
| LY9 | | SERPINA1 | | ZNF788 |
| MAG | | SGK | | |
| MAGED1 | | SGK1 | | |
| MAP1A | | SGSH | | |
| MAP4K4 | | SIDT2 | | |
| MAPKAPK2 | | SIGIRR | | |
| MAST3 | | SIGLEC14 | | |
| MCM6 | | SIGLEC16 | | |
| MCOLN2 | | SIGLEC9 | | |
| MDC1 | | SIPA1L2 | | |
| MEF2D | | SIRPA | | |
| MEX3B | | SLC11A1 | | |
| MGAT4A | | SLC16A5 | | |
| MGC29506 | | SLC22A16 | | |
| MGC33556 | | SLC22A11 | | |
| MGC39900 | | SLC27A3 | | |
| MGC42367 | | SLC2A3 | | |
| MIB2 | | SLC31A2 | | |
| MIR155HG | | SLC40A1 | | |
| MKNK1 | | SLC46A2 | | |
| MLL4 | | SLC7A7 | | |
| MME | | SLITRK4 | | |
| MMP11 | | SMAGP | | |
| MMP23B | | SMAP2 | | |
| MMRN1 | | SMARCD3 | | |
| MNAT1 | | SNRK | | |
| MOXD1 | | SNTB1 | | |
| MPEG1 | | SRBD1 | | |
| MRPL36 | | SRGAP3 | | |
| MS4A4A | | ST3GAL6 | | |
| MSRB3 | | STEAP3 | | |

TABLE 3
List of anti-human antibodies used for
mass cytometry (CyTOF).

| Metal | Name | Clone | Company | Cell expression |
|---|---|---|---|---|
| 89 | CD45 | HI30 | Fluidigm | all leukocytes |
| 112/ | CD14 | TUK4 | Invitrogen | monocytes |
| 114 | | | | |
| 115 | CD15 | HI98 | Biolegend | PMN, monocytes |
| 141 | CD7 | 6B7 | Biolegend | T cells, NK cells |
| 142 | CD26 | BA26 | Biolegentd | cDC1 |
| 143 | CD62L | DREG-56 | Biolegentd | Lymphocytes, |
| | | | | monocytes, |
| | | | | granulocytes. |
| 144 | CD48 | BL40 | Biolegend | Lymphocytes, DCs |
| 145 | CD68 | KP1 | eBiosciene | pDC, mono/macro |
| 146 | CD5 | UCHT2 | Biolegend | cDC2 |
| 147 | CD86 | IT2.2 | BD Biosciences | DC |
| 148 | CD85j | 292319 | R&D | B cells, DCs, |
| | | | | monocytes, NK |
| | | | | and T cells |
| 149 | HLA-DR | L243 | BD Biosciences | APC |
| 150 | CD80 | L307.4 | BD Biosciences | DC |
| 151 | CADM1 | 3E1 | MBL | cDC1 |
| 152 | CD1c | L161 | Biolegend | cDC2 |
| 153 | FceR1 | AER-37 | eBioscience | cDC2 |
| 154 | CD327 | 767329 | R&D | pDC |
| 155 | CDI23 | 6H6 | BD Biosciences | pDC |
| 156 | CD163 | GHI | Biolegend | cDC2, mono |
| 157 | CXCR3 | 1C6 | BD Biosciences | cDC1 |
| 158 | CD56 | NCAM16.2 | BD Biosciences | NK cells, |
| | | | | DC subsets |
| 159 | CD33 | WM53 | BD Biosciences | myeloid cells |
| 160 | Clec9a | 683409 | R&D Systems | cDC1 |
| 161 | CD38 | HIT2 | Biolegend | HSCs, plasma |
| | | | | cells, NK cells T |
| | | | | and B cells |
| 162 | CD10 | HI10a | Biolegend | B cell precursors, |
| | | | | T cell |
| | | | | precursors, |
| | | | | PMN |
| 163 | BTLA | MIH26 | Fluidigm | cDC1, cDC2 |
| | | | | subset |
| 164 | CD141 | 1A4 | BD Biosciences | cDC1 |
| 165 | CD303 | 201A | Biolegend | pDC |
| 166 | CD16 | 3G8 | Biolegend | mono, NK cells |
| 167 | CX3CR1 | KO124E1 | Biolegend | cDC2, mono |
| 168 | CCR2 | KO36C2 | Biolegend | cDC, mono |
| 169 | CD116 | 4H11 | Biolegend | DC |
| 170 | CD19 | HIB19 | Biolegend | B cells |
| 171 | CD34 | 581 | Biolegend | HSC |
| 172 | CD2 | RPA-2.10 | Biolegend | cDC2 |
| 173 | CD13 | WM15 | Biolegend | cDC1 |
| 174 | CD45RA | HI100 | Biolegend | pDC |
| 175 | CD11c | B-Ly6 | BD Biosciences | cDc |
| 176 | CD11b | ICRF44 | Biolegend | cDC2 subset, |
| | | | | mono |

TABLE 4
Number of expressed genes detected per cell in the
pre-DC C1 scmRNAseq experiment.

| | Number of | Number | |
|---|---|---|---|
| | detected | of | detected |
| Cell ID | genes | Cell ID | genes |
| RMS641 | 4997 | RMS687 | 4667 |
| RMS642 | 5935 | RMS688 | 5199 |
| RMS643 | 4873 | RMS689 | 5320 |
| RMS644 | 5000 | RMS690 | 3683 |
| RMS645 | 3193 | RMS691 | 3816 |
| RMS646 | 3255 | RMS692 | 4366 |
| RMS647 | 2653 | RMS693 | 5400 |
| RMS648 | 5217 | RMS694 | 5018 |
| RMS649 | 5191 | RMS695 | 3457 |
| RMS650 | 5235 | RMS696 | 3660 |
| RMS651 | 4836 | RMS697 | 4845 |
| RMS652 | 5715 | RMS698 | 3945 |
| RMS653 | 5224 | RMS699 | 3801 |
| RMS654 | 4681 | RMS700 | 5533 |
| RMS655 | 4014 | RMS701 | 5089 |
| RMS656 | 4134 | RMS702 | 4365 |
| RMS657 | 4895 | RMS703 | 4462 |
| RMS658 | 5094 | RMS704 | 3770 |
| RMS659 | 5405 | RMS705 | 4897 |
| RMS660 | 3701 | RMS706 | 5048 |
| RMS661 | 4432 | RMS707 | 5435 |
| RMS662 | 3298 | RMS708 | 4930 |
| RMS663 | 3843 | RMS709 | 5308 |
| RMS664 | 4417 | RMS710 | 5067 |
| RMS665 | 5162 | RMS711 | 5536 |
| RMS666 | 4042 | RMS712 | 3275 |
| RMS667 | 5172 | RMS713 | 4810 |
| RMS668 | 5129 | RMS714 | 4878 |
| RMS669 | 3613 | RMS715 | 5270 |
| RMS670 | 3571 | RMS716 | 4324 |
| RMS671 | 5016 | RMS717 | 4130 |
| RMS672 | 5170 | RMS718 | 3840 |
| RMS673 | 4996 | RMS719 | 4134 |
| RMS674 | 5462 | RMS720 | 3592 |
| RMS675 | 4190 | RMS722 | 4461 |
| RMS676 | 5206 | RMS723 | 4804 |
| RMS677 | 5590 | RMS724 | 3950 |
| RMS678 | 3177 | RMS725 | 4062 |
| RMS679 | 3938 | RMS726 | 2551 |
| RMS680 | 1802 | RMS727 | 3749 |
| RMS681 | 3377 | RMS728 | 3574 |
| RMS682 | 4166 | RMS729 | 4247 |
| RMS683 | 3863 | RMS730 | 5363 |
| RMS684 | 4279 | RMS731 | 5072 |
| RMS685 | 5128 | RMS732 | 4992 |
| RMS686 | 4884 | RMS733 | 5301 |

TABLE 5
Lists of genes identified from the microarray
DEG analysis comparisons along the lineage
progression from early pre-DC to mature cDC,
for cDC1 and cDC2 respectively, and the list of
the 62 common genes.

| Profile Genes | Profile Genes | 62 common |
|---|---|---|
| cDC1 | cDC2 | elements |
| ABCA1 | ABHD8 | ACTN1 |
| ABCB9 | ACAD8 | ADAM33 |
| ABLIM1 | ACTN1 | ADAMTSL2 |
| ACAA1 | ADAM19 | ARHGAP22 |
| ACP5 | ADAM33 | AXL |
| ACP6 | ADAMTSL2 | BATF3 |
| ACSS1 | AGPAT9 | CARD11 |
| ACTN1 | AIF1 | CCDC50 |
| AGY3 | ANXA2P1 | CCND3 |
| ADAM33 | AOAH | CD22 |
| ADAMTSL2 | AP4M1 | CD52 |
| ADAP1 | APLP2 | CLEC4C |
| AIM1 | ARHGAP1 | CTSG |
| ALG5 | ARHGAP22 | CYP2S1 |
| ALOX5 | ARHGAP23 | CYP2S1 |
| ALOX5AP | AXL | EXT1 |
| AMICA1 | BACH2 | FCN1 |
| ANG | BATF3 | GPRC5C |
| ANPEP | BTBD11 | GPX7 |
| ANXA2 | C10ORF11 | GRINA |
| APOBEC3H | C10ORF84 | HAMP |
| APOL2 | C15ORF48 | HRASLS3 |
| APOL3 | C16ORF33 | HSPA12B |
| ARHGAP22 | C20ORF27 | ID2 |
| ARMET | C2ORF89 | IL3RA |
| ASB16 | C3ORF60 | IRAK3 |
| ASCL2 | CARD11 | KCNK10 |
| ATN1 | CCDC50 | LGALS3 |
| ATP2A1 | CCL3L1 | LILRA4 |
| AXL | CCND3 | LIME1 |
| B9D1 | CD1C | LIMS2 |
| BAIAP3 | CD1D | LOC387841 |
| BATF3 | CD1E | LOC387882 |
| B LK | CD207 | LOC392382 |
| BTLA | CD22 | LOC401720 |
| BUB3 | CD52 | LTK |
| C10ORF105 | CD81 | MARCKS |
| C11ORF80 | CD86 | MUPCDH |
| C15ORF39 | CEBPB | MYBPHL |
| C17ORF61 | CHST7 | NCLN |
| C19ORF10 | CLEC4C | OSBPL3 |
| C1ORF21 | CLIC3 | PLAC8 |
| C1ORF54 | COQ10A | PLP2 |
| C1RL | CREB5 | PPP1R14A |
| C20ORF100 | CSTA | RARRES3 |
| C9ORF91 | CTSG | RHOC |
| CACNA2D3 | CXCR3 | RPP21 |
| CADM1 | CYBASC3 | RTN1 |
| CALR | CYP2S1 | S100A9 |
| CAMK1G | DAB2 | SERPING1 |
| CAPN12 | DEF8 | SHD |
| CAPZB | DEK | SIGLEC6 |
| CARD11 | DEPDC6 | SLC15A2 |
| CASP1 | DFFB | SLC20A1 |
| CCDC123 | E2F7 | SLC44A2 |
| CCDC50 | ECE1 | STARD7 |
| CCNB2 | ELMO1 | STMN2 |
| CCND1 | ELOVL1 | TBC1D19 |
| CCND3 | EML4 | TCF4 |
| CD22 | EXT1 | TP53I11 |
| CD27 | FAM105A | ZBP1 |
| CD300LB | FAM1298 | ZFP36L1 |
| CD300LF | FAM179A | |
| CD38 | FAM26F | |
| CD5 | FBXL6 | |
| CD52 | FCGBP | |
| CD68 | FCGR2A | |
| CD7 | FCNI | |
| CD79A | FCRLA | |
| CD79B | FLJ22662 | |
| CDC20 | GADD45B | |
| CDC25B | GBP1 | |
| CDC45L | GPRC5C | |
| CDH1 | GPX7 | |
| CDH2 | GRINA | |
| CDKN1A | HAMP | |
| CDS1 | HAPLN3 | |
| CECR1 | HK2 | |
| CENPM | HLA-DPB1 | |
| CLEC10A | HLA-DQB1 | |
| CLEC4C | HRASLS3 | |
| CLEC9A | HSPAI2B | |
| CLNK | HSPA7 | |
| CMTM3 | HTR3A | |
| COL18A1 | ID2 | |
| COMMD4 | IL13RA1 | |
| CPNE3 | IL3RA | |
| CPNE5 | IRAK3 | |
| CPVL | IRF8 | |
| CRKRS | ITGAL | |
| CSF1R | JDP2 | |
| CSRP1 | KCNK10 | |
| CTSG | LAT2 | |
| CXCL16 | LCNL1 | |
| CYP2E1 | LGALS3 | |
| CYP2S1 | LHFPL2 | |
| DAB2 | LILRA4 | |
| DAPK1 | LIME1 | |
| DBN1 | LIME2 | |
| DEX1 | LIPT1 | |
| DIAPH3 | LOC100134361 | |
| DUS3L | LOC339352 | |
| DUSP3 | LOC387841 | |
| DYSF | LOC387882 | |
| EAF2 | LOC389816 | |
| EEF1A2 | LOC392382 | |
| ENO1 | LOC401720 | |
| ENPP1 | LOC440280 | |
| EPPB9 | LOC642299 | |
| EXT1 | LOC642367 | |
| FAIM3 | LOC644879 | |
| FAM160A2 | LOC728069 | |
| FAM30A | LOC729406 | |
| FAR2 | LOXL3 | |
| FBLN2 | LRP1 | |
| FCER1A | LRP5 | |
| FCER1G | LRRC26 | |
| FCN1 | LTK | |
| FER1L4 | MADD | |
| FERMT3 | MARCKS | |
| FIS1 | MBNL1 | |
| FKBP11 | MEFV | |
| FKBP1B | MIIP | |
| FLJ40504 | MUPCDH | |
| FNDC3B | MYB | |
| GANC | MYBPHL | |
| GAS6 | MYL6B | |
| GDPD5 | NCKAP1L | |
| GEMIN6 | NCLN | |
| GGTL3 | NOXA1 | |
| GLDC | NRP1 | |
| GMPPB | NTAN1 | |
| GPER | OGFRL1 | |
| GPR162 | OLFM1 | |
| GPRC5C | OSBPL3 | |
| GPRC5D | PACSIN1 | |
| GPS2 | PAK1 | |
| GPX7 | PARP10 | |
| GRINA | PCBP1 | |
| GZMK | PCP4L1 | |
| H2AFY2 | PCSK4 | |
| HAMP | PHYHD1 | |
| HCST | PILRA | |
| HEXIM1 | PLAC8 | |
| HK3 | PLOD3 | |
| HLA-DOB | PLP2 | |
| HN1 | POLR2I | |
| HOPX | PPM1J | |
| HRASLS2 | PPP1R14A | |
| HRASLS3 | PPP1R14B | |
| HSH2D | PROC | |
| HSPA12B | PTGS2 | |
| HSPA8 | PTGS2 | |
| HVCN1 | RAB20 | |
| ID2 | RAB7L1 | |
| IDH2 | RARRES3 | |
| IDO1 | RASSF4 | |
| IGJ | RHOC | |
| IGLL1 | RILPL2 | |
| IGLL3 | RPP21 | |
| IL3RA | RS1 | |
| IL7R | RTN1 | |
| INDO | S100A8 | |
| IRAK2 | S100A9 | |
| IRAK3 | SCMH1 | |
| IRF2BP2 | SCN9A | |
| IRF4 | SERPINA1 | |
| ISCU | SERPINF1 | |
| ISG20 | SERPING1 | |
| ITM2C | SGK | |
| ITPR3 | SGK1 | |
| JARID2 | SHANK3 | |
| KCNK10 | SHD | |
| KCNK12 | SIGLEC6 | |
| KIAA0101 | SLAMF7 | |
| KIAA0114 | SLC15A2 | |
| KIAA1191 | SLC20A1 | |
| KIAA1545 | SLC2A8 | |
| KIT | SLC35C2 | |
| KLF6 | SLC44A2 | |
| KRTI8P13 | SMARCD3 | |
| L2HGDH | SOX4 | |
| LAMP1 | SP140 | |
| LGALS3 | SPOCK2 | |
| LGALS8 | SSR1 | |
| LILRA2 | STARD7 | |
| LILRA4 | STARD8 | |
| LILRB2 | STMN2 | |
| LILRB4 | TBC1D19 | |
| LIME1 | TCF4 | |
| LIMS2 | TCL1A | |
| LMNA | TMEM14C | |
| LOC100130171 | TMEM2 | |
| LOC100130367 | TP53I11 | |
| LOC100130856 | TREM1 | |
| LOC100131727 | TRIB2 | |
| LOC100132444 | TSPAN13 | |
| LOC144383 | TXNIP | |
| LOC286076 | USP24 | |
| LOC387841 | VASN | |
| LOC387882 | VCAN | |
| LOC392382 | VEGFB | |
| LOC399988 | VENTX | |
| LOC401720 | VSIG4 | |
| LOC642113 | ZAK | |
| LOC642755 | ZBP1 | |
| LOC645381 | ZFP36L1 | |
| LOC647506 | ZNF469 | |
| LOC648366 | ZNF503 | |
| LOC649210 | | |
| LOC649923 | | |
| LOC652493 | | |
| LOC652694 | | |
| LOC653468 | | |
| LOC653566 | | |
| LOC654191 | | |
| LOC728014 | | |
| LOC728093 | | |
| LOC728557 | | |
| LOC729086 | | |
| LPXN | | |
| LST1 | | |
| LTK | | |
| LYN | | |
| MARCKS | | |
| MBOAT2 | | |
| MBOAT7 | | |
| MCM4 | | |
| MED12L | | |
| MED27 | | |
| MEI1 | | |
| MGC13057 | | |
| MGC29506 | | |
| MGC33556 | | |
| MIF | | |
| MIR939 | | |
| MIST | | |
| MLKL | | |
| MS4A6A | | |
| MUPCDH | | |
| MYBPHL | | |
| MYO1D | | |
| MYO5C | | |
| NADK | | |
| NAV1 | | |
| NCF4 | | |
| NCLN | | |
| NDRG1 | | |
| NDRG2 | | |
| NFATC2IP | | |
| NGFRAP1 | | |
| NLRC3 | | |
| NRM | | |
| NRSN2 | | |
| NT5DC2 | | |
| NUBP1 | | |
| NUCB2 | | |
| OSBPL10 | | |
| OSBPL3 | | |
| PARM1 | | |
| PARP3 | | |
| PCNA | | |
| PDE9A | | |
| PDIA4 | | |
| PEPD | | |
| PIK3CD | | |
| PLAC8 | | |
| PLCD1 | | |
| PLD3 | | |
| PLEKHG2 | | |
| PLP2 | | |
| PLXNB2 | | |
| PMS2L4 | | |
| POP5 | | |
| POU2AF1 | | |
| PPM1H | | |
| PPP1R14A | | |
| PRDM1 | | |
| PRDX4 | | |
| PRKCZ | | |
| PRKD2 | | |
| PRR5 | | |
| PRSSL1 | | |
| PSEN2 | | |
| PSMB8 | | |
| PSORS1C1 | | |
| PTGER2 | | |
| PTTG1 | | |
| PTTG3P | | |
| RAB30 | | |
| RAB32 | | |
| RAB43 | | |
| RARRES3 | | |
| RASGRP2 | | |
| RASSF2 | | |
| RHBDF2 | | |
| RHOC | | |
| RNF130 | | |
| RNU6-15 | | |
| RPP21 | | |
| RPSI9BP1 | | |
| RPS27L | | |
| RTN1 | | |
| RUFY1 | | |
| S100A4 | | |
| S100A9 | | |
| SAMD3 | | |
| SCPEP1 | | |
| SDF2L1 | | |
| SEC11C | | |
| SEMA4C | | |
| SEPT3 | | |
| SERPINF2 | | |
| SERPING1 | | |
| SH2D3A | | |
| SHD | | |
| SHE | | |
| SHMT2 | | |
| SIAH1 | | |
| SIGLEC6 | | |
| SLC15A2 | | |
| SLC15A3 | | |
| SLC20A1 | | |
| SLC25A4 | | |
| SLC35A5 | | |
| SLC41A2 | | |
| SLC44A1 | | |
| SLC44A2 | | |
| SLC9A3R1 | | |
| SLCO3A1 | | |
| SMO | | |
| SNCA | | |
| SNN | | |
| SNX22 | | |
| SNX29 | | |
| SNX3 | | |
| SPATS2 | | |
| SSR4 | | |
| ST6GALNAC2 | | |
| STARD5 | | |
| STARD7 | | |
| STMN2 | | |
| SULF2 | | |
| SUSD3 | | |
| TACSTD2 | | |
| TBC1D19 | | |
| TCF4 | | |
| TDRD1 | | |
| TFPI | | |
| TGM2 | | |
| TLR3 | | |
| TMEM109 | | |
| TMEM167B | | |
| TMEM216 | | |
| TMEM97 | | |
| TNFRSF13B | | |
| TNFRSFI7 | | |
| TNFRSF21 | | |
| TNFSF12 | | |
| TNNI2 | | |
| TOP2A | | |
| TOX2 | | |
| TP53I11 | | |
| TP53INP1 | | |
| TRIB1 | | |
| TRPM2 | | |
| TSEN34 | | |
| TSEN54 | | |
| TSPAN33 | | |
| TSPYL1 | | |
| TUFT1 | | |
| TXNDC5 | | |
| TYMS | | |
| TYROBP | | |
| UBE2C | | |
| UBXN11 | | |
| UGCGL2 | | |
| UNC119 | | |
| UNG | | |
| VAC14 | | |
| VISA | | |
| VPS37B | | |
| VPS37D | | |
| WDFY4 | | |
| WDR34 | | |
| WFS1 | | |
| WWC3 | | |
| XBP1 | | |
| ZBP1 | | |
| ZBTB32 | | |
| ZFP36L1 | | |
| ZNF662 | | |
| ZNF821 | | |

TABLE 6
List of anti-human antibodies used for flow cytometry.

| Name | Clone | Fluorophore | Source |
|---|---|---|---|
| CADM1 | 3E1 | Purified | MBL |
| CD116 | 4H1 | Biotion | Biolegend |
| CD117 | 104D2 | BV421 | Biolegend |
| CD11c | B-ly6 | V450 | BD Biosciences |
| CD11c | 3.9 | BV605 | Biolegend |
| CD123 | 7G3 | BUV395 | BD Biosciences |
| CD123 | 6H6 | PercP/Cy5.5 | BD Biosciences |
| CD135 | 4G8 | PE | BD Pharmigen |
| CD135 | 4G8 | BV711 | BD Biosciences |
| CD14 | RMO52 | ECD | Beckman Coulter |
| CD14 | M5E2 | BV711 | Biolegend |
| CD14 | M5E2 | BV650 | BD Biosciences |
| CD141 | AD5-14H12 | PE/Vio770 | Miltenyi Biotec |
| CD16 | 3G8 | APC/Cy7 | Biolegend |
| CD16 | 3G8 | BV650 | BD Biosciences |
| CD169 | 7-239 | PE | BD Biosciences |
| CD172α | SE5a5 | PECy7 | Biolegend |
| CD183 | 1C6/CXCR3 | APC | BD Biosciences |
| CD19 | SJ25C1 | BV650 | BD Biosciences |
| CD1c | L161 | PercP/Cy5.5 | Biolegond |
| CD1c | L161 | PE/Cy7 | Biolegend |
| CD1c | L16I | APC/Cy7 | Biolegend |
| CD2 | RPA-2.10 | BV421 | BD Biosciences |
| CD20 | 2H7 | BV650 | BD Biosciences |
| CD22 | HIB22 | BV421 | BD Biosciences |
| CD26 | BA5b | PE/Cy7 | Biolegend |
| CD272 | MIH26 | PE | Biolegend |
| CD283 | 40C1285.6 | PE | Abcam |
| CD289 | J15A7 | PE | BD Biosciences |
| CD3 | SP34-2 | BV650 | BD Biosciences |
| CD303 | AC144 | Biotin | Miltenyi Biotec |
| CD319 | 162.1 | PE | Biolegend |
| CD327 | 767329 | APC | R&D Systems |
| CD33 | WM53 | PE-CF594 | BD Biosciences |
| CD33 | AC104.3E3 | VioBlue | Miltenyi Biotec |
| CD33 | P67.6 | PercP/Cy5.5 | BD Biosciences |
| CD335 | 9E2 | PerCP5.5 | Biolegend |
| CD34 | 581 | Alexa Fluor 700 | BD Biosciences |
| CD40 | 5C3 | PercP/Cy5.5 | Biolegend |
| CD45 | HI30 | V500 | BD Biosciences |
| CD45RA | 5H9 | FITC | BD Biosciences |
| CD45RA | L48 | PE/Cy7 | BD Biosciences |
| CD5 | UCHT2 | BB515 | BD Biosciences |
| CD66b | G10F5 | PerCP5.5 | Biolegend |
| CD7 | 124-1D1 | PE | eBioscience |
| CD80 | ASL24 | PE | Biolegend |
| CD80 | 2D10 | BV421 | Biolegend |
| CD83 | HB15e | PE | Biolegend |
| CD86 | 2331 (FUN-1) | Biotin | BD Biosciences |
| CD88 | S5/1 | PE/Cy7 | Biolegend |
| Clec9a | 8F9 | APC | Biolegend |
| Clec9A | 3A4/C1ec9A | PE | BD Biosciences |
| CX3CR1 | 2A9-1 | PE | Biolegend |
| CX3CR1 | K0124E1 | PE | Biolegend |
| CXCR3 | G025H7 | PE | Biolegend |
| FcεRIα | AER-37 | PerCP | Biolegend |
| FcεRIα | AER-37 | PE | Biolegend |
| HLA-DR | L243 | BV605 | Biolegend |
| HLA-DR | L243 | BV785 | Biolegend |
| IFNα | LT27:295 | FITC | Miltenyi Biotec |
| IL-12p40 | C8.6 | BV421 | BD Biosciences |
| ILT1 | REA219 | Biotin | Miltenyi Biotec |
| ILT3 | ZM4.1 | PE | Biolegend |
| IRF4 | 3E4 | PE | eBioscience |
| IRF8 | V3GYWCH | PercP/eFluor710 | eBioscience |
| TLR7 | A94B10 | PE | BD Biosciences |
| TNFα | Mab11 | Alexa Flour 700 | BD Biosciences |
| secondary | reagents: | | |
| Live/Dead blue | | equ DAPI | Life Technologies |
| Streptavidin | | BUV737 | BD Biosciences |
| Chicken IgY | | Alexa Fluor 647 | Jackson |
| | | | Immunoresearch |

### DETAILED DESCRIPTION OF THE DRAWINGS

### Examples

Non-limiting examples of the invention will be further described in greater detail by reference to specific Examples, which should not be construed as in any way limiting the scope of the invention.

### Example 1—Methods

Blood, Bone Marrow and Spleen Samples

Human samples were obtained in accordance with a favorable ethical opinion from Singapore SingHealth and National Health Care Group Research Ethics Committees. Written informed consent was obtained from all donors according to the procedures approved by the National University of Singapore Institutional Review Board and SingHealth Centralised Institutional Review Board. Peripheral blood mononuclear cells (PBMC) were isolated by Ficoll-Paque (GE Healthcare) density gradient centrifugation of apheresis residue samples obtained from volunteer donors through the Health Sciences Authorities (HSA, Singapore). Blood samples were obtained from 4 patients with molecularly confirmed Pitt-Hopkins syndrome (PHS), who all showed the classical phenotype (1). Spleen tissue was obtained from patients with tumors in the pancreas who underwent distal pancreatomy (Singapore General Hospital, Singapore). Spleen tissue was processed as previously described (2). Bone marrow mononuclear cells were purchased from Lonza.

Generation of Single Cell Transcriptomes Using MARS-Seq

MARS-Seq using the Biomek FXP system (Beckman Coulter) as previously described (3) was performed for scmRNAseq of the DC compartment of the human peripheral blood. In brief, Lineage marker (Lin)(CD3/14/16/19/20/34)⁻CD45⁺CD135⁺HLA-DR⁺CD123⁺CD33⁺ single cells were sorted into individual wells of 384-well plates filled with 2 μl lysis buffer (Triton 0.2% (Sigma Aldrich) in molecular biology grade H₂O (Sigma Aldrich), supplemented with 0.4 U/μ1 protein-based RNase inhibitor (Takara Bio Inc.), and barcoded using 400 nM IDT. Details regarding the barcoding procedure with poly-T primers were previously described (3). Samples were pre-incubated for 3 min at 80° C. and reverse transcriptase mix consisting of 10 mM DTT (Invitrogen), 4 mM dNTPs (NEB), 2.5 U/μl SuperScript III Reverse Transcriptase (Invitrogen) in 50 mM Tris-HCl (pH 8.3; Sigma), 75 mM KCl (Sigma), 3 mM MgCl₂ (Sigma), ERCC RNA Spike-In mix (Life Technologies), at a dilution of 1:80*10⁷ per cell was added to each well. The mRNA was reverse-transcribed to cDNA with one cycle of 2 min at 42° C., 50 min at 50° C., and 5 min at 85° C. Excess primers were digested with ExoI (NEB) at 37° C. for 30 min then 10 min at 80° C., followed by cleanup using SPRIselect beads at a 1.2× ratio (Beckman Coulter). Samples were pooled and second strands were synthesized using a Second Strand Synthesis kit (NEB) for 2.5 h at 16° C., followed by a cleanup using SPRIselect beads at a 1.4× ratio (Beckman Coulter). Samples were linearly amplified by T7-promoter guided in vitro transcription using the T7 High Yield RNA polymerase IVT kit (NEB) at 37° C. for 12 h. DNA templates were digested with Turbo DNase I (Ambion) for 15 min at 37° C., followed by a cleanup with SPRIselect beads at a 1.2× ratio (Beckman Coulter). The RNA was then fragmented in Zn²⁺ RNA Fragmentation Solution (Ambion) for 1.5 min at 70° C., followed by cleanup with SPRIselect beads at a 2.0 ratio (Beckman Coulter). Barcoded ssDNA adapters (IDT; details of barcode see (3)) were then ligated to the fragmented RNAs in 9.5% DMSO (Sigma Aldrich), 1 mM ATP, 20% PEG8000 and 1 U/μl T4 RNA ligase I (NEB) solution in 50 mM Tris HCl pH7.5 (Sigma Aldrich), 10 mM MgCl₂ and 1 mM DTT for 2 h at 22° C. A second reverse transcription reaction was then performed using Affinity Script Reverse Transcription buffer, 10 mM DTT, 4 mM dNTP, 2.5 U/μl Affinity Script Reverse Transcriptase (Agilent) for one cycle of 2 min at 42° C., 45 min at 50° C., and 5 min at 85° C., followed by a cleanup on SPRIselect beads at a 1.5× ratio (Beckman Coulter). The final libraries were generated by subsequent nested PCR reactions using 0.5 μM of each Illumina primer (IDT; details of primers see (3)) and KAPA HiFi HotStart Ready Mix (Kapa Biosystems) for 15 cycles according to manufacturer's protocol, followed by a final cleanup with SPRIselect beads at a 0.7× ratio (Beckman Coulter). The quality and quantity of the resulting libraries was assessed using an Agilent 2200 TapeStation instrument (Agilent), and libraries were subjected to next generation sequencing using an Illumina HiSeq1500 instrument (PE no index; read1: 61 reads (3 reads random nucleotides, 4 reads pool barcode, 53 reads sequence), read2: 13 reads (6 reads cell barcode, 6 reads unique molecular identifier)).

Pre-Processing, Quality Assessment and Control of MARS-Seq Single Cell Transcriptome Data

Cell specific tags and Unique Molecular Identifiers (UMIs) were extracted (2,496 cells sequenced) from sequenced data-pool barcodes. Sequencing reads with ambiguous plate and/or cell-specific tags, UMI sequences of low quality (Phred <27), or reads that mapped to E. coli were eliminated using Bowtie 1 sequence analysis software (4), with parameters “-M 1 -t --best --chunkmbs 64 -strata”. Fastq files were demultiplexed using the fastx_barcode_splitter from fastx_toolkit, and R1 reads (with trimming of pooled barcode sequences) were mapped to the human hg38+ERCC pseudo genome assembly using Bowtie “-m 1 -t --best --chunkmbs 64 -strata”. Valid reads were then counted using UMIs if they mapped to the exon-based gene model derived from the BiomaRt HG38 data mining tool provided by Ensembl (46). A gene expression matrix was then generated containing the number of UMIs for every cell and gene. Additionally, UMIs and cell barcode errors were corrected and filtered as previously described (3).

Normalization and Filtering of MARS-Seq Single Cell Transcriptome Data

In order to account for differences in total molecule counts per cell, a down-sampling normalization was performed as suggested by several studies (3, 5). Here, every cell was randomly down-sampled to a molecule count of 1,050 unique molecules per cell (threshold details discussed below). Cells with molecule counts <1,050 were excluded from the analysis (Table 1: number of detected genes per cell). Additionally, cells with a ratio of mitochondrial versus endogenous genes exceeding 0.2, and cells with <90 unique genes, were removed from the analysis. Prior to Seurat analysis (4), expression tables were filtered to exclude mitochondrial and ribosomal genes to remove noise.

Analysis of MARS-Seq Single Cell Transcriptome Data

Analysis of the normalized and filtered single-cell gene expression data (8,657 genes across 710 single cell transcriptomes used in the final expression table) was achieved using Mpath (6), PCA, tSNE, connectivity MAP (cMAP) (7) and several functions of the Seurat single cell analysis package. cMAP analysis was performed using DEGs between pDC and cDC derived from the gene expression omnibu data series GSE35457 (2). For individual cells, cMAP generated enrichment scores that quantified the degree of enrichment (or “closeness”) to the given gene signatures. The enrichment scores were scaled and assigned positive or negative values to indicate enrichment for pDC or cDC signature genes, respectively. A permutation test (n=1,000) between gene signatures was performed on each enrichment score to determine statistical significance. For the tSNE/Seurat analysis, a Seurat filter was used to include genes that were detected in at least one cell (molecule count=1), and excluded cells with <90 unique genes. To infer the structure of the single-cell gene expression data, a PCA was performed on the highly variable genes determined as genes exceeding the dispersion threshold of 0.75. The first two principle components were used to perform a tSNE that was combined with a DBSCAN clustering algorithm (8) to identify cells with similar expression profiles. DBSCAN was performed by setting 10 as the minimum number of reachable points and 4.1 as the reachable epsilon neighbourhood parameter; the latter was determined using a KNN plot integrated in the DBSCAN R package (9) (https://cran.r-project.org/web/packages/dbscan/). The clustering did not change when using the default minimal number of reachable points.

To annotate the clusters, the gene signatures of blood pDC, cDC1 and cDC2 were derived from the Gene Expression Omnibus data series GSE35457 (2) (Table 2: lists of signature genes, data processing described below) to calculate the signature gene expression scores of cell type-specific gene signatures, and then these signature scores were overlaid onto the tSNE plots. Raw expression data of CD141⁺ (cDC1), CD1c⁺ (cDC2) DCs and pDC samples from blood of up to four donors (I, II, V and VI) was imported into Partek® Genomics Suite® software, version 6.6 Copyright©; 2017 (PGS), where they were further processed. Data were quantile-normalized and log 2-transformed, and a batch-correction was performed for the donor using PGS. Differential probe expression was calculated from the normalized data (ANOVA, Fold-Change ≥2 and FDR-adj. p-value <0.05) for the three comparisons of every cell type against the remaining cell types. The three lists of differentially-expressed (DE) probes were intersected and only exclusively-expressed probes were used for the cell-type specific gene signatures. The probes were then reduced to single genes, by keeping the probe for a corresponding gene with the highest mean expression across the dataset. Resulting gene signatures for blood pDCs, CD1c⁺ and CD141⁺ DCs contained 725, 457 and 368 genes, respectively. The signature gene expression score was calculated as the mean expression of all signature genes in a cluster. In order to avoid bias due to outliers, the trimmed mean (trim=0.08) was calculated.

Monocle analysis was performed using the latest pre-published version of Monocle v.2.1.0 (10). The data were loaded into a monocle object and then log-transformed. Ordering of the genes was determined by dispersion analysis if they had an average expression of ≥0.5 and at least a dispersion of two times the dispersion fit. The dimensionality reduction was performed using the reduceDimension command with parameters max_components=2, reduction_method=“DDRTree” and norm_method=“log”. The trajectory was then built using the plot_cell_trajectory command with standard parameters.

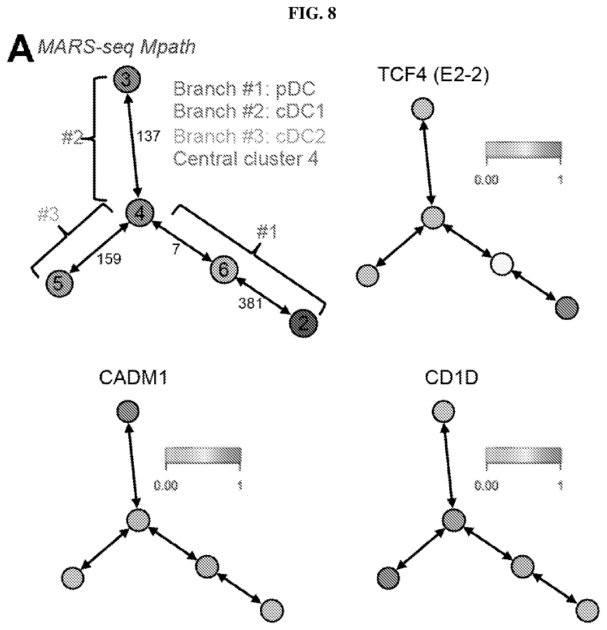

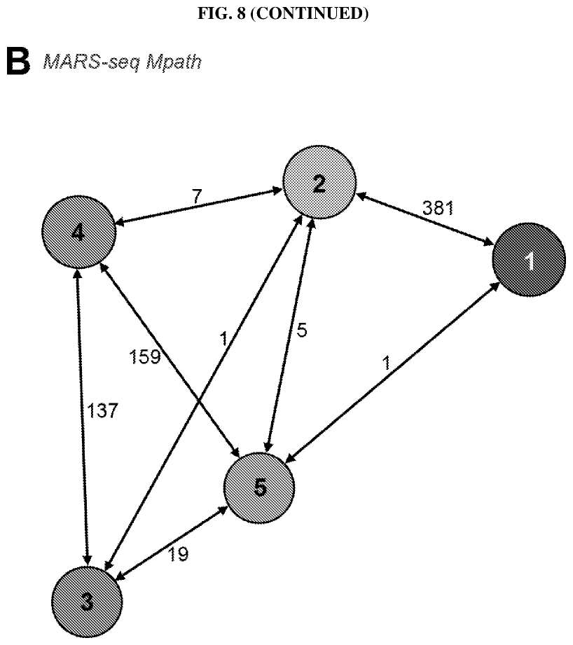

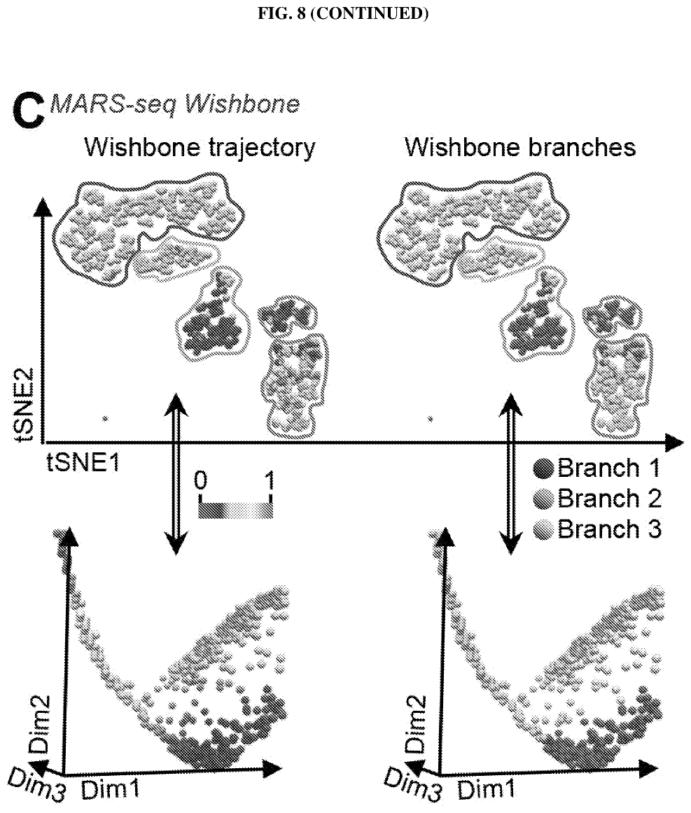

Wishbone analysis (11) was performed using the Python toolkit downloaded from https://github.com/ManuSetty/wishbone. MARS-seq data were loaded using the wishbone.wb.SCData.from_csv function with the parameters data_type=′sc-seq′ and normalize=True. Wishbone was then performed using wb.run_wishbone function with parameter start_cell=“run1_CATG_AAGACA”, components_list=[1, 2, 3, 4], num_waypoints=150, branch=True. Start_cell was randomly selected from the central cluster #4. Diffusion map analysis was performed using the scdata.run_diffusion_map function with default parameters (12). Wishbone revealed three trajectories giving rise to pDC, cDC1 and cDC2 respectively. Along each trajectory, the respective signature gene shows increasing expression (FIG. 8C). Although Wishbone results might be interpreted to suggest that cDC2 are early cells and differentiate into pDC and cDC1 on two separate branches, this is simply because Wishbone allows a maximum of two branches and assumes all cells fall on continuous trajectories. Nevertheless, it is able to delineate the three trajectories that are in concordance with Mpath, monocle, and diffusion map analysis.

C1 Single Cell mRNA Sequencing

Lin(CD3/14/16/19/20)⁻HLA-DR⁺CD33⁺CD123⁺ cells at 300 cells/μl were loaded onto two 5-10 μm C1 Single-Cell Auto Prep integrated fluidic circuits (Fluidigm) and cell capture was performed according to the manufacturer's instructions. Individual capture sites were inspected under a light microscope to confirm the presence of single, live cells. Empty capture wells and wells containing multiple cells or cell debris were discarded for quality control. A SMARTer Ultra Low RNA kit (Clontech) and Advantage 2 PCR Kit (Clontech) was used for cDNA generation. An ArrayControl™ RNA Spots and Spikes kit (with spike numbers 1, 4 and 7) (Ambion) was used to monitor technical variability, and the dilutions used were as recommended by the manufacturer. The concentration of cDNA for each single cell was determined by Quant-iT™ PicoGreen® dsDNA Reagent, and the correct size and profile was confirmed using DNA High Sensitivity Reagent Kit and DNA Extended Range LabChip (Perkin Elmer). Multiplex sequencing libraries were generated using the Nextera XT DNA Library Preparation Kit and the Nextera XT Index Kit (Illumina). Libraries were pooled and subjected to an indexed PE sequencing run of 2×51 cycles on an Illumina HiSeq 2000 (Illumina) at an average depth of 2.5-million row reads/cell.

C1 Single Cell Analysis

Raw reads were aligned to the human reference genome GRCh38 from GENCODE (13) using RSEM program version 1.2.19 with default parameters (14). Gene expression values in transcripts per million were calculated using the RSEM program and the human GENCODE annotation version 22. Quality control and outlier cell detection was performed using the SINGuLAR (Fluidigm) analysis toolset. cMAP analysis was performed using cDC1 versus cDC2 DEGs identified from Gene Expression Omnibus data series GSE35457 (2), and the enrichment scores were obtained. Similar to the gene set enrichment analyses, cMAP was used to identify associations of transcriptomic profiles with cell-type characteristic gene signatures.

Mpath Analysis of MARS- or C1 Single Cell mRNA Sequencing Data

Developmental trajectories were defined using the Mpath algorithm (6), which constructs multi-branching cell lineages and re-orders individual cells along the branches. In the analysis of the MARS-seq single cell transcriptomic data, the Seurat R package was first used to identify five clusters: for each cluster, Mpath calculated the centroid and used it as a landmark to represent a canonical cellular state; subsequently, for each single cell, Mpath calculated its Euclidean distance to all the landmarks, and identified the two nearest landmarks. Each individual cell was thus assigned to the neighborhood of its two nearest landmarks. For every pair of landmarks, Mpath then counted the number of cells that were assigned to the neighborhood, and used the determined cell counts to estimate the possibility of the transition between landmarks to be true. A high cell count implied a high possibility that the transition was valid. Mpath then constructed a weighted neighborhood network whereby nodes represented landmarks, edges represented a putative transition between landmarks, and numbers allocated to the edges represented the cell-count support for the transition. Given that single cell transcriptomic data tend to be noisy, edges with low cell-count support were considered likely artifacts. Mpath therefore removed the edges with a low cell support by using (0-n) (n-n represents cell count) to quantify the distance between nodes followed by applying a minimum spanning tree algorithm to find the shortest path that could connect all nodes with the minimum sum of distance. Consequently, the resulting trimmed network is the one that connects all landmarks with the minimum number of edges and the maximum total number of cells on the edges. Mpath was then used to project the individual cells onto the edge connecting its two nearest landmarks, and assigned a pseudo-time ordering to the cells according to the location of their projection points on the edge. In the analysis of the C1 single cell transcriptome data, the cMAP analysis was first used to identify cDC1-primed, un-primed, and cDC2-primed clusters, and then Mpath was used to construct the lineage between these three clusters. The Mpath analysis was carried out in an un-supervised manner without prior knowledge of the starting cells or number of branches. This method can be used for situations of non-branching networks, bifurcations, and multi-branching networks with three or more branches.

Mass Cytometry Staining, Barcoding, Acquisition and Data Analysis

For mass cytometry, pre-conjugated or purified antibodies were obtained from Invitrogen, Fluidigm (pre-conjugated antibodies), Biolegend, eBioscience, Becton Dickinson or R&D Systems as listed in Table 3. For some markers, fluorophore- or biotin-conjugated antibodies were used as primary antibodies, followed by secondary labeling with anti-fluorophore metal-conjugated antibodies (such as the anti-FITC clone FIT-22) or metal-conjugated streptavidin, produced as previously described (15). Briefly, 3×10⁶ cells/well in a U-bottom 96 well plate (BD Falcon, Cat #3077) were washed once with 200 μL FACS buffer (4% FBS, 2 mM EDTA, 0.05% Azide in 1×PBS), then stained with 100 μL 200 μM cisplatin (Sigma-Aldrich, Cat #479306-1G) for 5 min on ice to exclude dead cells. Cells were then incubated with anti-CADM1-biotin and anti-CD19-FITC primary antibodies in a 50 μL reaction for 30 min on ice. Cells were washed twice with FACS buffer and incubated with 50 μL heavy-metal isotope-conjugated secondary mAb cocktail for 30 min on ice. Cells were then washed twice with FACS buffer and once with PBS before fixation with 200 μL 2% paraformaldehyde (PFA; Electron Microscopy Sciences, Cat #15710) in PBS overnight or longer. Following fixation, the cells were pelleted and resuspended in 200 uL 1× permeabilization buffer (Biolegend, Cat #421002) for 5 mins at room temperature to enable intracellular labeling. Cells were then incubated with metal-conjugated anti-CD68 in a 50 μL reaction for 30 min on ice. Finally, the cells were washed once with permeabilization buffer and then with PBS before barcoding.

Bromoacetamidobenzyl-EDTA (BABE)-linked metal barcodes were prepared by dissolving BABE (Dojindo, Cat #B437) in 100 mM HEPES buffer (Gibco, Cat #15630) to a final concentration of 2 mM. Isotopically-purified PdCl2 (Trace Sciences Inc.) was then added to the 2 mM BABE solution to a final concentration of 0.5 mM. Similarly, DOTA-maleimide (DM)-linked metal barcodes were prepared by dissolving DM (Macrocyclics, Cat #B-272) in L buffer (MAXPAR, Cat #PN00008) to a final concentration of 1 mM. RhCl₃ (Sigma) and isotopically-purified LnCl₃ was then added to the DM solution at 0.5 mM final concentration. Six metal barcodes were used: BABE-Pd-102, BABE-Pd-104, BABE-Pd-106, BABE-Pd-108, BABE-Pd-110 and DM-Ln-113.

All BABE and DM-metal solution mixtures were immediately snap-frozen in liquid nitrogen and stored at −80° C. A unique dual combination of barcodes was chosen to stain each tissue sample. Barcode Pd-102 was used at 1:4000 dilution, Pd-104 at 1:2000, Pd-106 and Pd-108 at 1:1000, Pd-110 and Ln-113 at 1:500. Cells were incubated with 100 μL barcode in PBS for 30 min on ice, washed in permeabilization buffer and then incubated in FACS buffer for 10 min on ice. Cells were then pelleted and resuspended in 100 μL nucleic acid Ir-Intercalator (MAXPAR, Cat #201192B) in 2% PFA/PBS (1:2000), at room temperature. After 20 min, cells were washed twice with FACS buffer and twice with water before a final resuspension in water. In each set, the cells were pooled from all tissue types, counted, and diluted to 0.5×10⁶ cells/mL. EQ Four Element Calibration Beads (DVS Science, Fluidigm) were added at a 1% concentration prior to acquisition. Cell data were acquired and analyzed using a CyTOF Mass cytometer (Fluidigm).

The CyTOF data were exported in a conventional flow-cytometry file (.fcs) format and normalized using previously-described software (16). Events with zero values were randomly assigned a value between 0 and −1 using a custom R script employed in a previous version of mass cytometry software (17). Cells for each barcode were deconvolved using the Boolean gating algorithm within FlowJo. The CD45⁺Lin (CD7/CD14/CD15/CD16/CD19/CD34)⁻HLA-DR⁺ population of PBMC were gated using FlowJo and exported as a .fcs file. Marker expression values were transformed using the logicle transformation function (18). Random sub-sampling without replacement was performed to select 20,000 cell events. The transformed values of sub-sampled cell events were then subjected to t-distributed Stochastic Neighbor Embedding (tSNE) dimension reduction (19) using the markers listed in Table 3, and the Rtsne function in the Rtsne R package with default parameters. Similarly, isometric feature mapping (isoMAP) (20) dimension reduction was performed using vegdist, spantree and isomap functions in the vegan R package (21).

The vegdist function was run with method=“euclidean”. The spantree function was run with default parameters. The isoMAP function was run with ndim equal to the number of original dimensions of input data, and k=5. Phenograph clustering (22) was performed using the markers listed in Table 3 before dimension reduction, and with the number of nearest neighbors equal to 30. The results obtained from the tSNE, isoMAP and Phenograph analyses were incorporated as additional parameters in the .fcs files, which were then loaded into FlowJo to generate heat plots of marker expression on the reduced dimensions. The above analyses were performed using the cytofkit R package which provides a wrapper of existing state-of-the-art methods for cytometry data analysis (23).

Human Cell Flow Cytometry: Labeling, Staining, Analysis and Cell Sorting

All antibodies used for fluorescence-activated cell sorting (FACS) and flow cytometry were mouse anti-human monoclonal antibodies (mAbs), except for chicken anti-human CADM1 IgY primary mAb. The mAbs and secondary reagents used for flow cytometry are listed in Table 6. Briefly, 5×10⁶ cells/tube were washed and incubated with Live/Dead blue dye (Invitrogen) for 30 min at 4° C. in phosphate buffered saline (PBS) and then incubated in 5% heat-inactivated fetal calf serum (FCS) for 15 min at 4° C. (Sigma Aldrich). The appropriate antibodies diluted in PBS with 2% fetal calf serum (FCS) and 2 mM EDTA were added to the cells and incubated for 30 min at 4° C., and then washed and detected with the secondary reagents. For intra-cytoplasmic or intra-nuclear labeling or staining, cells were fixed and permeabilized with BD Cytofix/Cytoperm (BD Biosciences) or with eBioscience FoxP3/Transcription Factor Staining Buffer Set (eBioscience/Affimetrix), respectively according to the manufacturer's instructions. Flow cytometry was performed using a BD LSRII or a BD FACSFortessa (BD Biosciences) and the data analyzed using BD FACSDiva 6.0 (BD Biosciences) or FlowJo v.10 (Tree Star Inc.). For isolation of precursor dendritic cells (pre-DC), PBMC were first depleted of T cells, monocytes and B cells with anti-CD3, anti-CD14 and anti-CD20 microbeads (Miltenyi Biotec) using an AutoMACS Pro Separator (Miltenyi Biotec) according to the manufacturer's instructions. FACS was performed using a BD FACSAriaII or BD FACSAriaIII (BD Biosciences). Wanderlust analysis (33) of flow cytometry data was performed using the CYT tool downloaded from https://www.c2b2.columbia.edu/danapeerlab/html/cyt-download.html. As Wanderlust requires users to specify a starting cell, one cell was selected at random from the CD45RA⁺CD123⁺ population.

Cytospin and Scanning Electron Microscopy

Cytospins were prepared from purified cells and stained with the Hema 3 system according to the manufacturer's protocol (Fisher Diagnostics). Images were analyzed at 100× magnification with an Olympus BX43 upright microscope (Olympus). Scanning electron microscopy was performed as previously described (2).

Dendritic Cell (DC) Differentiation Co-Culture Assay on MS-5 Stromal Cells

MS-5 stromal cells were maintained and passaged as previously described (24). MS-5 cells were seeded in 96-well round-bottom plates (Corning) at 3,000 cells per well in complete alpha-Minimum Essential Media (α-MEM) (Life Technologies) supplemented with 10% fetal bovine serum (FBS) (Serana) and 1% penicillin/streptomycin (Nacalai Tesque). A total of 5,000 sorted purified cells were added 18-24 h later, in medium containing 200 ng/mL Flt3L (Miltenyi Biotec), 20 ng/mL SCF (Miltenyi Biotec), and 20 ng/mL GM-CSF (Miltenyi Biotec), and cultured for up to 5 days. The cells were then resuspended in their wells by physical dissociation and filtered through a cell strainer into a polystyrene FACS tube.

Intracellular Cytokine Detection Following Stimulation with TLR Ligands

A total of 5×10⁶ PBMC were cultured in Roswell Park Memorial Institute (RPMI)-1640 Glutmax media (Life Technologies) supplemented with 10% FBS, 1% penicillin/streptomycin and stimulated with either lipopolysaccharide (LPS, 100 ng/mL; InvivoGen), LPS (100 ng/mL)+interferon gamma (IFNγ, 1,000 U/mL; R&D Systems), Flagellin (100 ng/mL, Invivogen), polyI:C (10 μg/mL; InvivoGen), Imidazoquinoline (CL097; Invivogen) or CpG oligodeoxynucleotides 2216 (ODN, 5 μM; InvivoGen) for 2 h, after which 10 μg/ml Brefeldin A solution (eBioscience) was added and the cells were again stimulated for an additional 4 h. After the 6 h stimulation, the cells were labeled with cytokine-specific antibodies and analyzed by flow cytometry, as described above.

Mixed Lymphocyte Reaction

Naïve T cells were isolated from PBMC using Naïve Pan T-Cell Isolation Kit (Miltenyi Biotec) according to the manufacturer's instructions, and labeled with 0.2 μM carboxyfluorescein succinimidyl ester (CFSE) (Life Technologies) for 5 min at 37° C. A total of 5,000 cells from sorted DC subsets were co-cultured with 100,000 CFSE-labeled naïve T cells for 7 days in Iscove's Modified Dulbecco's Medium (IMDM; Life Technologies) supplemented with 10% KnockOut™ Serum Replacement (Life Technologies). On day 7, the T cells were stimulated with 10 μg/ml phorbol myristate acetate (InvivoGen) and 500 μg/ml ionomycin (Sigma Aldrich) for 1 h at 37° C. 10 μg/ml Brefeldin A solution was added for 4 h, after which the cells were labeled with cytokine-specific antibodies and analyzed by flow cytometry, as described above.

Electron Microscopy

Sorted cells were seeded on poly-lysine-coated coverslips for 1 h at 37° C. The cells were then fixed in 2% glutaraldehyde in 0.1 M cacoldylate buffer, pH 7.4 for 1 h, post fixed for 1 h with 2% buffered osmium tetroxide, then dehydrated in a graded series of ethanol solutions, before embedding in epoxy resin. Images were acquired with a Quemesa (SIS) digital camera mounted on a Tecnai 12 transmission electron microscope (FEI Company) operated at 80 kV.

Microarray Analysis

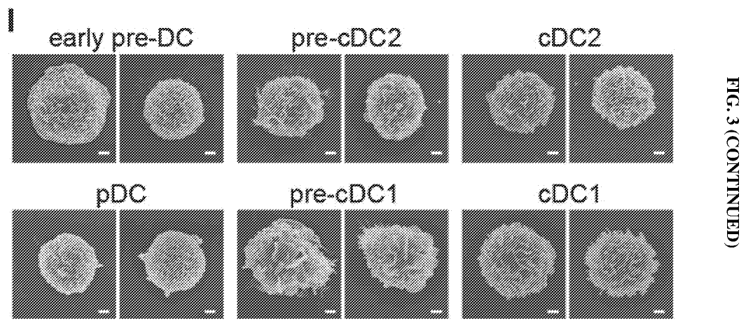

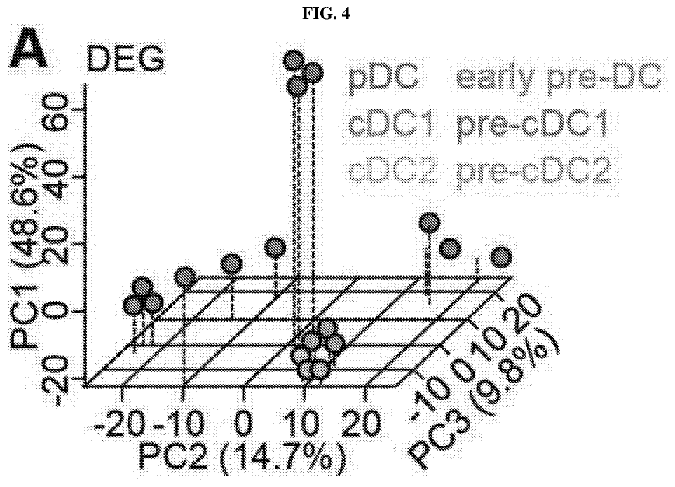

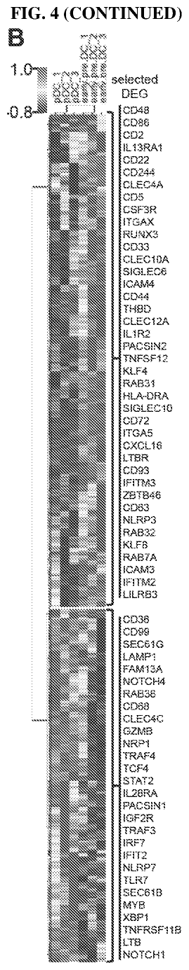

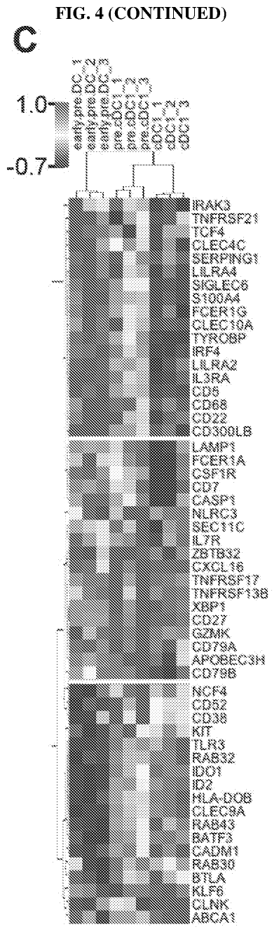

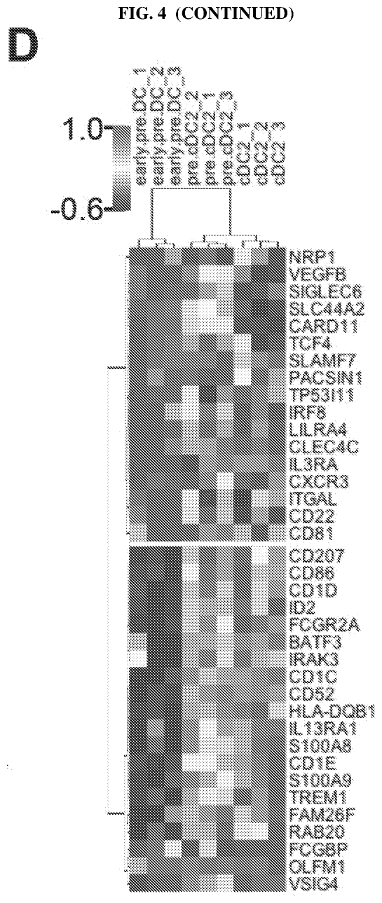

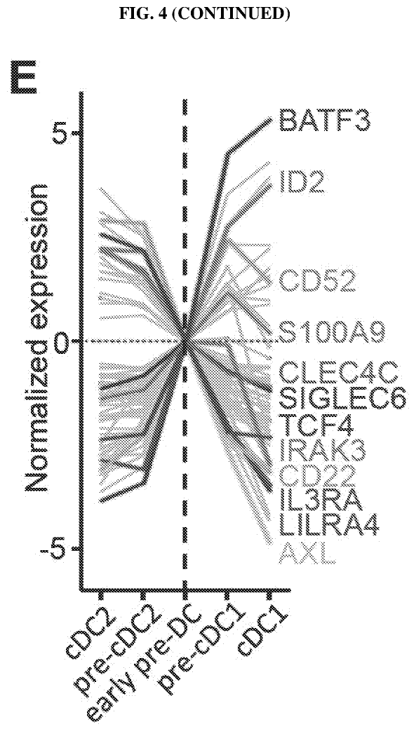

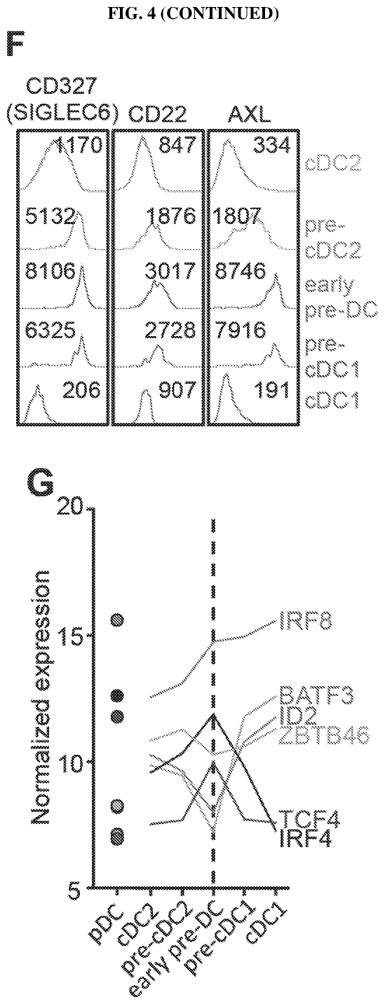

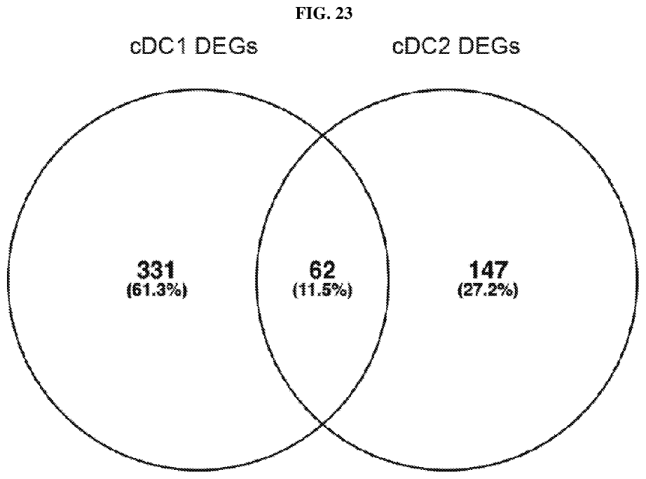

Total RNA was isolated from FACS-sorted blood pre-DC and DC subsets using a RNeasy® Micro kit (Qiagen). Total RNA integrity was assessed using an Agilent Bioanalyzer (Agilent) and the RNA Integrity Number (RIN) was calculated. All RNA samples had a RIN ≥7.1. Biotinylated cRNA was prepared using an Epicentre TargetAmp™ 2-Round Biotin-aRNA Amplification Kit 3.0 according to the manufacturer's instructions, using 500 pg of total RNA starting material. Hybridization of the cRNA was performed on an Illumina Human-HT12 Version 4 chip set (Illumina). Microrarray data were exported from GenomeStudio (Illumina) without background subtraction. Probes with detection P-values >0.05 were considered as not being detected in the sample, and were filtered out. Expression values for the remaining probes were logₑ transformed and quantile normalized. For differentially-expressed gene (DEG) analysis, comparison of one cell subset with another was carried out using the limma R software package (25) with samples paired by donor identifiers. DEGs were selected with Benjamini-Hochberg multiple testing (26) corrected P-value <0.05. In this way, limma was used to select up and down-regulated signature genes for each of the cell subsets in the pre-DC data by comparing one subset with all other subsets pooled as a group. Expression profiles shown in FIG. 4E were from 62 common genes identified from the union of DEGs from comparing pre-cDC1 versus early pre-DC and cDC1 versus pre-cDC1, and the union of DEGs from comparing pre-cDC2 versus early pre-DC and cDC2 versus pre-cDC2 (see Table 5 for the lists of DEGs for cDC1 lineage and cDC2 lineage, and the lists of the 62 common genes; FIG. 23 for Venn diagram comparison of the two lists of DEGs and identification of the 62 common genes).

Luminex® Drop Array™ Assay on Sorted and Stimulated Pre-DC and DC Populations

A total of 2,000 cells/well of sorted pre-DC and DC subsets were seeded in V-bottom 96 well plates and then incubated for 18 h in 50 μL complete RPMI-1640 Glutmax media (Life Technologies) supplemented with 10% FBS and 1% penicillin/streptomycin, and stimulated with either LPS, LPS+IFNγ, Flagellin, polyI:C, Imidazoquinoline or CpG oligodeoxynucleotides (ODN) 2216. Cells were then pelleted and 30 μL supernatant was collected. A Luminex® Drop Array™ was performed using 5 μL of the supernatant. Human G-CSF, GM-CSF, IFN-α2, IL-10, IL-12p40, IL-12p70, IL-15, IL-1RA, IL-1a, IL-1b, IL-6, IL-7, IL-8, MIP-1b, TNF-α, TNF-β were tested by multiplexing (EMD Millipore) with DropArray-bead plates (Curiox) according to the manufacturer's instructions. Acquisition was performed using xPONENT 4.0 (Luminex) acquisition software, and data analysis was performed using Bio-Plex Manager 6.1.1 (Bio-Rad).

Statistical Analyses

The Mann-Whitney test was used to compare data derived from patients with Pitt-Hopkins Syndrome and controls and the intracellular detection of IL-12p40 and TNF-α in pre-DC stimulated with LPS or poly I:C versus CpG ODN 2216. The Kruskal-Wallis test, followed by the Dunn's multiple comparison test, was used to compare the expression level of individual genes in single cells in the MARS-seq single cell RNAseq dataset. Differences were defined as statistically significant when adjusted P<0.05. All statistical tests were performed using GraphPad Prism 6.00 for Windows (GraphPad Software). Correlation coefficients were calculated as Pearson's correlation coefficient.

### Example 2—Results

Unbiased Identification of DC Precursors by Unsupervised Single-Cell RNAseq and CyTOF

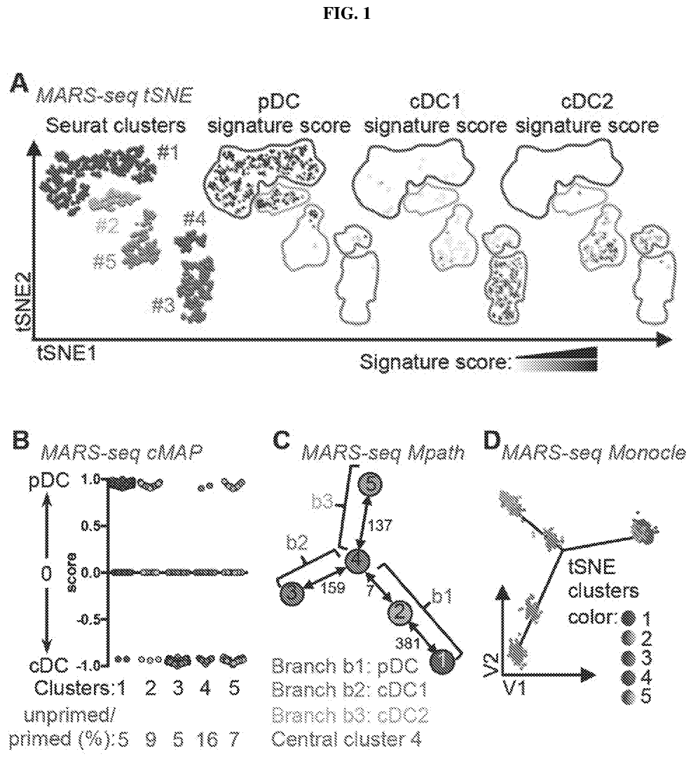

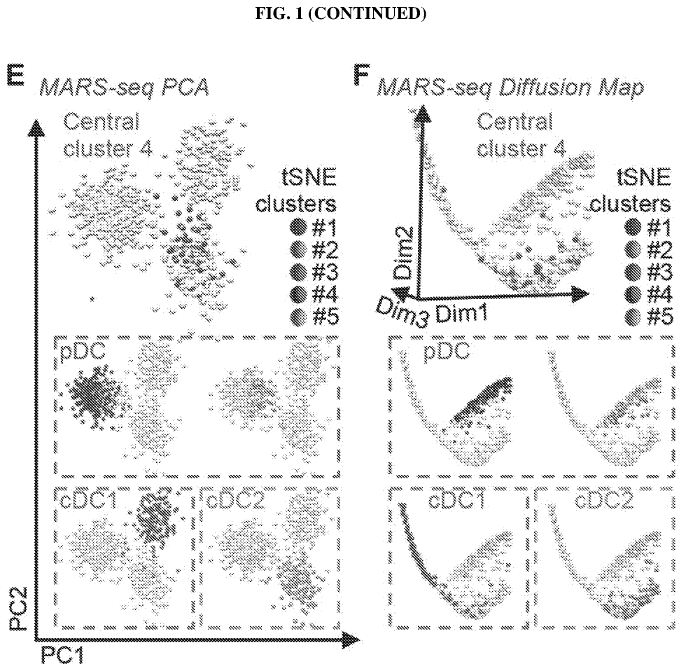

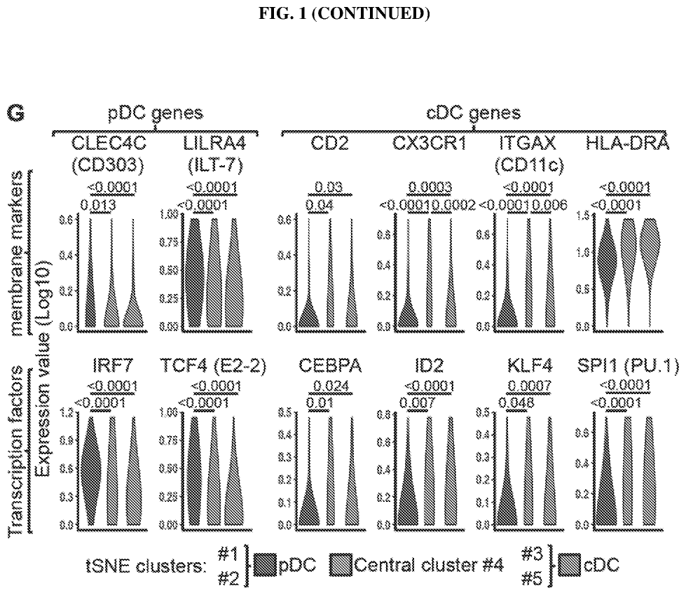

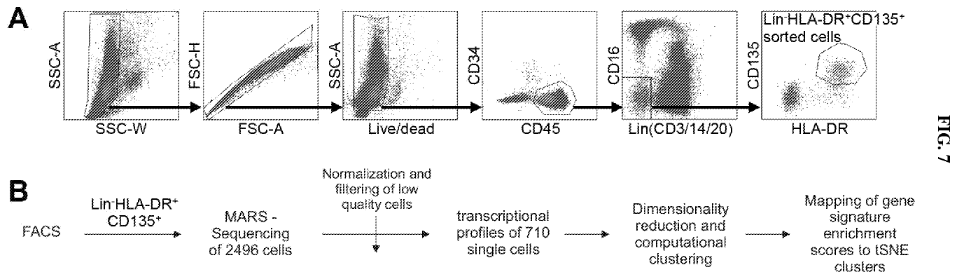

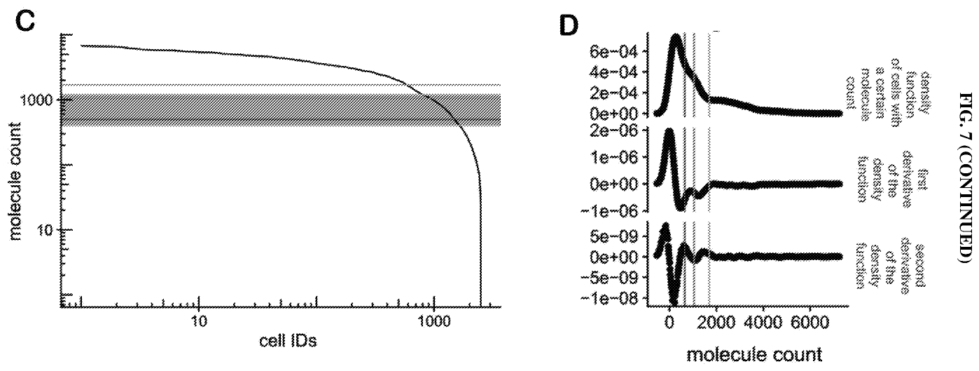

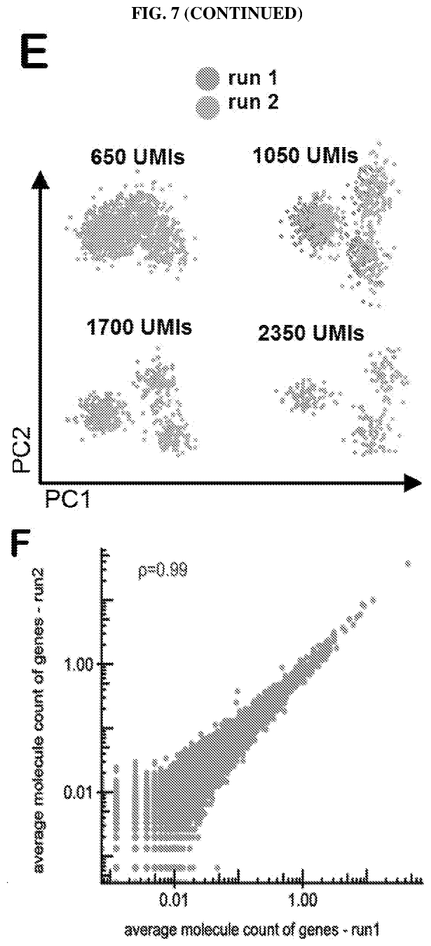

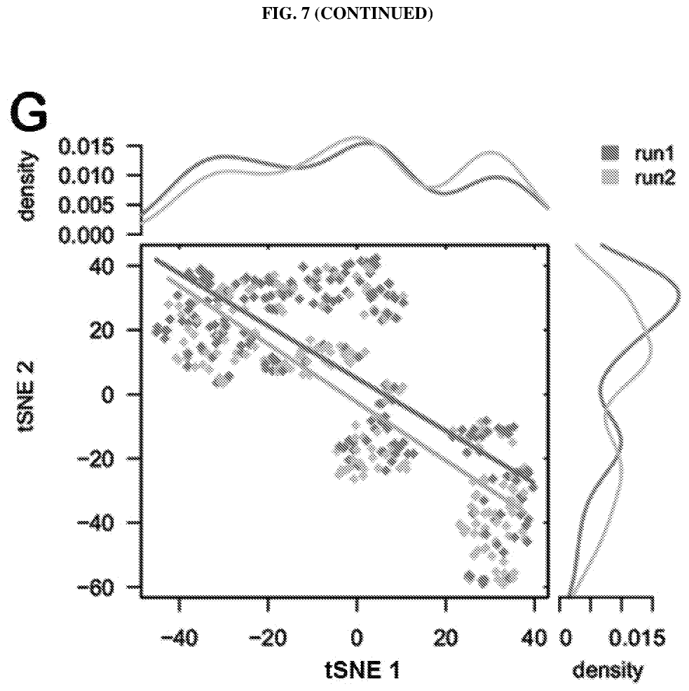

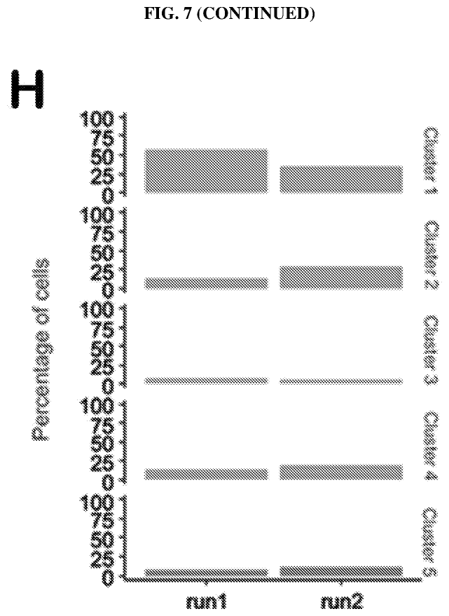

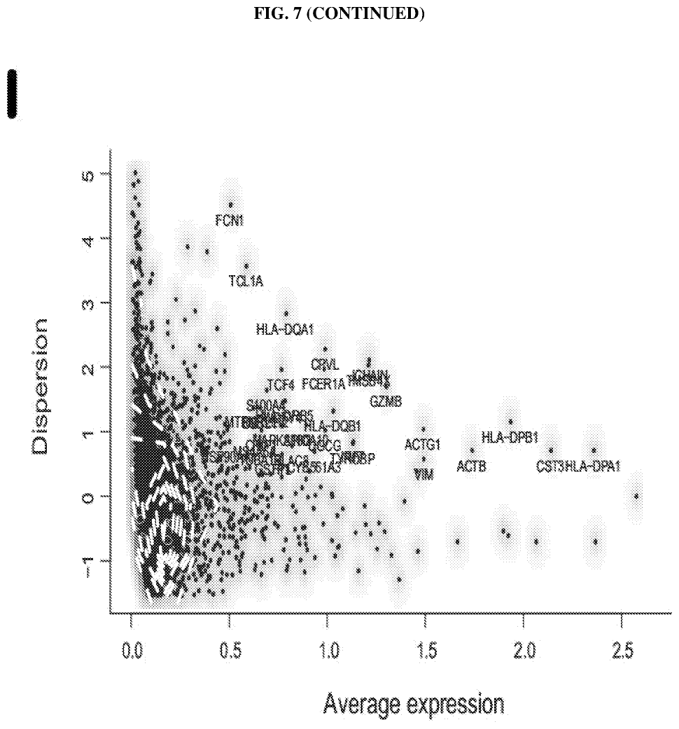

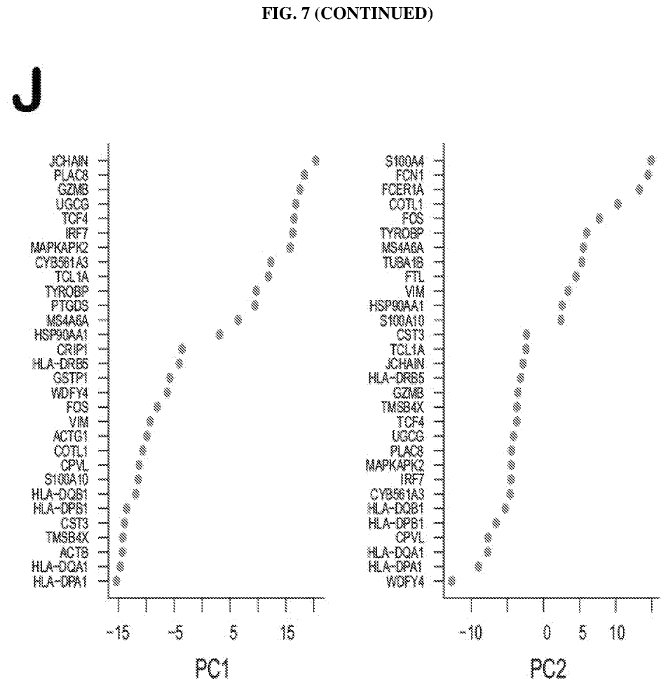

Using PBMC isolated from human blood, massively-parallel single-cell mRNA sequencing (MARS-seq) (3) was performed to assess the transcriptional profile of 710 individual cells within the lineage marker (Lin)(CD3/CD14/CD16/CD20/CD34)⁻, HLA-DR⁺CD135⁺ population (FIG. 1, A to G, and FIG. 7A: sorting strategy, FIG. 7, B to J: workflow and quality control, Table 1: number of detected genes). The MARS-seq data were processed using non-linear dimensionality reduction via t-stochastic neighbor embedding (tSNE), which enables unbiased visualization of high-dimensional similarities between cells in the form of a two-dimensional map (15, 27, 19). Density-based spatial clustering of applications with noise (DBSCAN) (8) on the tSNE dimensions identified five distinct clusters of transcriptionally-related cells within the selected PBMC population (FIG. 1A, and FIG. 7G). To define the nature of these clusters, gene signature scores were calculated for pDC, cDC1 and cDC2 (as described in (2), Table 2: lists of signature genes), and the expression of the signatures attributed to each cell was overlaid onto the tSNE visualization. Clusters #1 and #2 (containing 308 and 72 cells, respectively) were identified as pDC, cluster #3 (containing 160 cells) was identified as cDC1, and cluster #5 (containing 120 cells) was identified as cDC2. Cluster #4 (containing 50 cells) laid in between the cDC1 (#3) and cDC2 (#5) clusters and possessed a weak, mixed pDC/cDC signature (FIG. 1A). A connectivity MAP (cMAP) analysis (7) was employed to calculate the degree of enrichment of pDC or cDC signature gene transcripts in each individual cell. This approach confirmed the signatures of pDC (#1 and #2) and cDC (#3 and #5) clusters, and showed that most cells in cluster #4 expressed a cDC signature (FIG. 1B).

The Mpath algorithm (6) was then applied to the five clusters to identify hypothetical developmental relationships based on these transcriptional similarities between cells (FIG. 1C, and FIGS. 8, A and B). Mpath revealed that the five clusters were grouped into three distinct branches with one central cluster (cluster #4) at the intersection of the three branches (FIG. 1C, and FIG. 8A). The Mpath edges connecting cluster #4 to cDC1 cluster #3 and cDC2 cluster #5 have a high cell count (159 and 137 cells, respectively), suggesting that the transition from cluster #4 to clusters #3 and #5 is likely valid, and indicates that cluster #4 could contain putative cDC precursors (FIG. 1C). In contrast, the edge connecting cluster #4 and pDC cluster #2 has a cell count of only 7 (FIG. 1C, and FIG. 8B), which suggests that this connection is very weak. The edge connecting cluster #4 and #2 was retained when Mpath trimmed the weighted neighborhood network (FIG. 8B), simply due to the feature of the Mpath algorithm that requires all clusters to be connected (6). Monocle (10), principal component analyses (PCA), Wishbone (11) and Diffusion Map algorithms (12) were used to confirm these findings. Monocle and PCA resolved the cells into the same three branches as the original Mpath analysis, with the cells from the tSNE cluster #4 again falling at the intersection (FIGS. 1, D and E). Diffusion Map and Wishbone analyses indicated that there was a continuum between clusters #3 (cDC1), #4 and #5 (cDC2): cells from cluster #4 were predominantly found in the DiffMap_dim2^{low} region, and cells from clusters #3 and #5 were progressively drifting away from the DiffMap_dim2^{low} region towards the left and right, respectively. The pDC clusters (#1 and #2) were clearly separated from all other clusters (FIG. 1F, and FIG. 8C). In support of this observation, cells from these pDC clusters had a higher expression of pDC-specific markers and transcription factors (TF) than the cDC clusters (#3 and #5) and central cluster #4. Conversely, cells in cluster #4 expressed higher levels of markers and TF associated with all cDC lineage than the pDC clusters (FIG. 1G). This points to the possibility that cluster #4 represented a population of putative uncommitted cDC precursors.

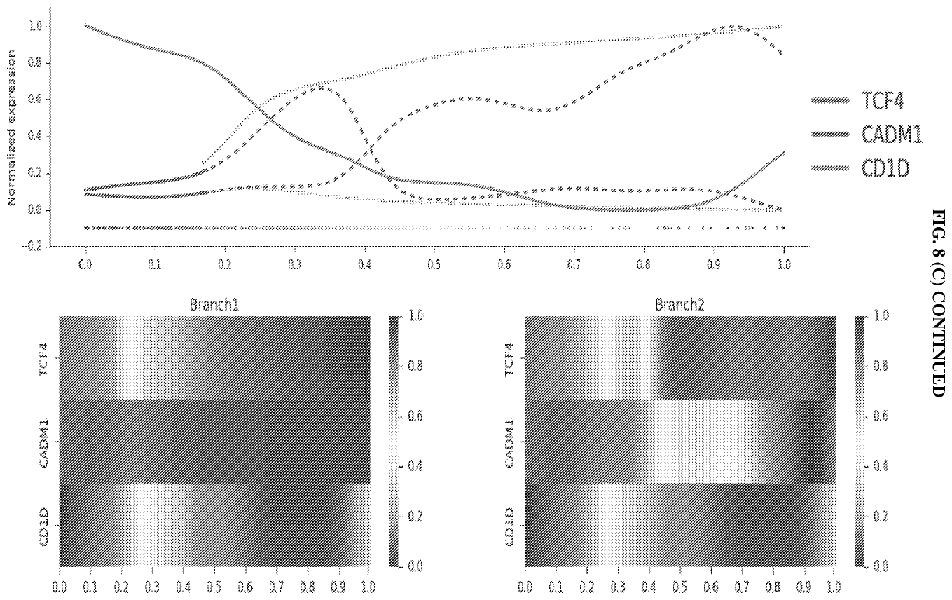

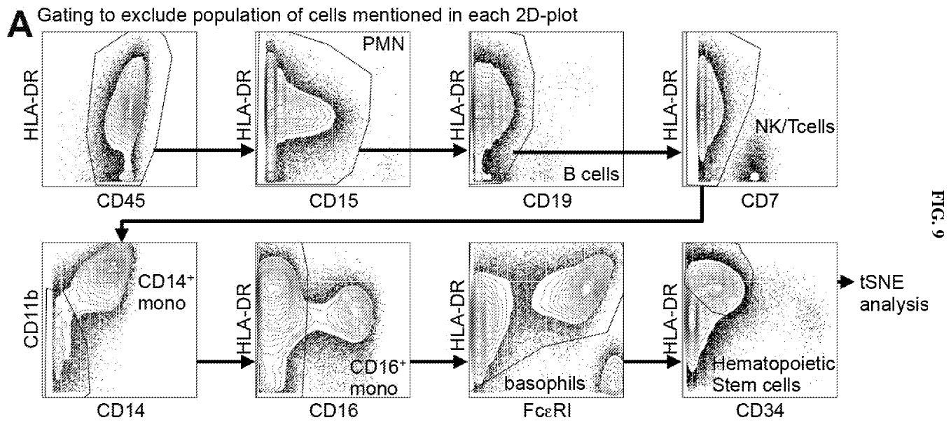

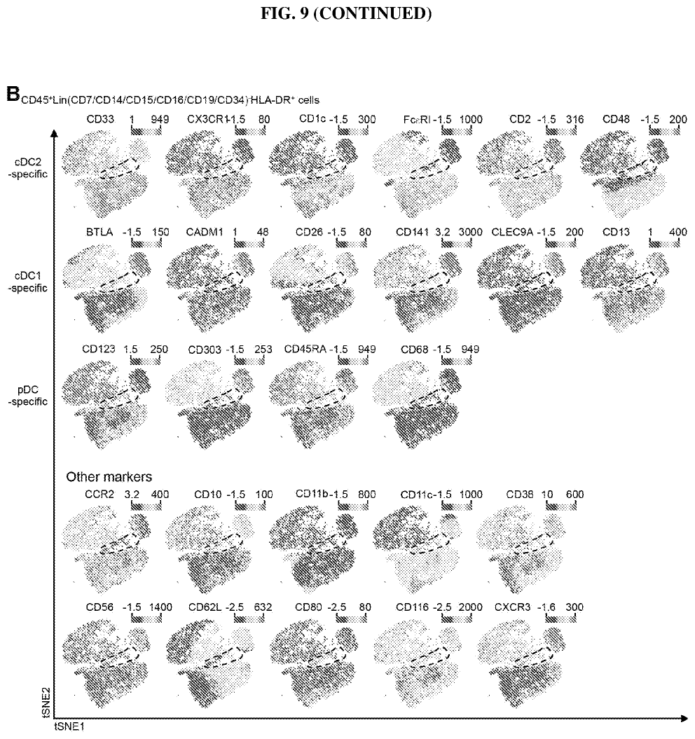

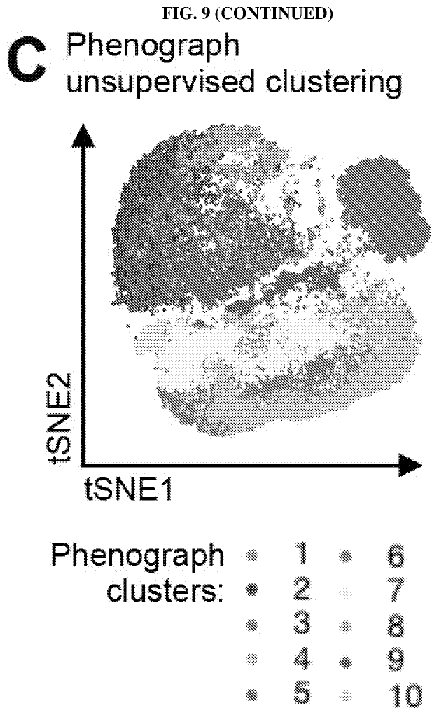

Next, CyTOF, which simultaneously measures the intensity of expression of up to 38 different molecules at the single cell level, was employed to further understand the composition of the delineated sub-populations. A panel of 38 labeled antibodies were designed to recognize DC lineage and/or progenitor-associated surface molecules (Table 3, FIG. 1, H to J, and FIG. 9), and the molecules identified in cluster #4 by MARS-seq, such as CD2, CX3CR1, CD11c and HLA-DR (FIG. 1I). Using the tSNE algorithm, the CD45⁺Lin(CD7/CD14/CD15/CD16/CD19/CD34)⁻HLA-DR⁺PBMC fraction (FIG. 9A) resolved into three distinct clusters representing cDC1, cDC2 and pDC (FIG. 1H). An intermediate cluster at the intersection of the cDC and pDC clusters that expressed both cDC-associated markers (CD11c/CX3CR1/CD2/CD33/CD141/BTLA) and pDC-associated markers (CD45RA/CD123/CD303) (FIG. 1, I to J, and FIG. 9B) corresponded to the MARS-seq cluster #4. The delineation of these clusters was confirmed when applying the phenograph unsupervised clustering algorithm (22) (FIG. 9C). The position of the intermediate CD123⁺CD33⁺ cell cluster was distinct, and the cells exhibited high expression of CD5, CD327, CD85j, together with high levels of HLA-DR and the cDC-associated molecule CD86 (FIG. 1, I to J). Taken together, these characteristics raise the question of whether CD123⁺CD33⁺ cells might represent circulating human pre-DC.

Pre-DC Exist within the pDC Fraction and Give Rise to cDC

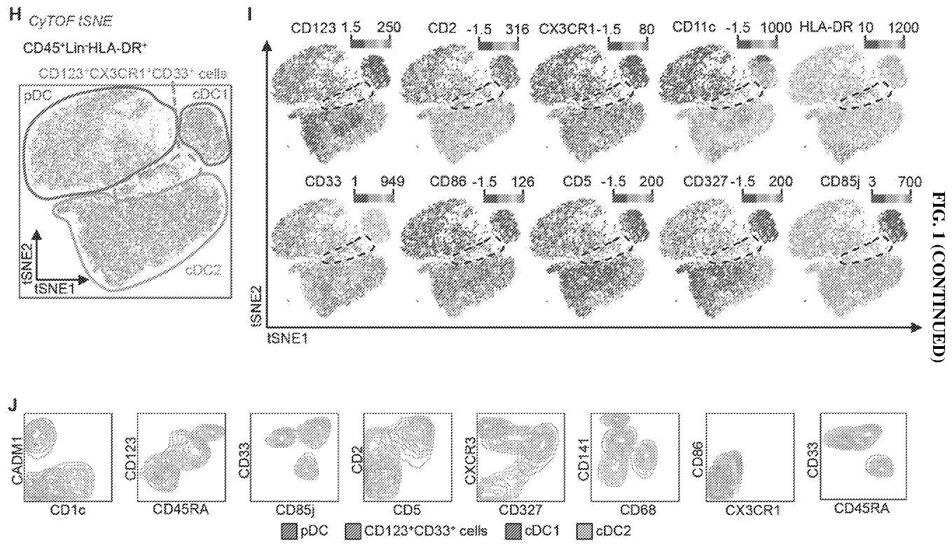

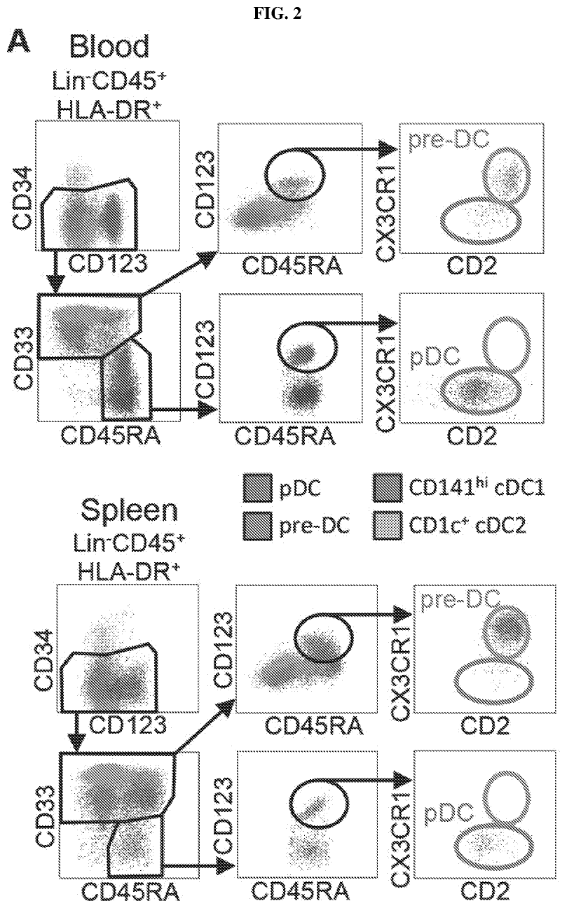

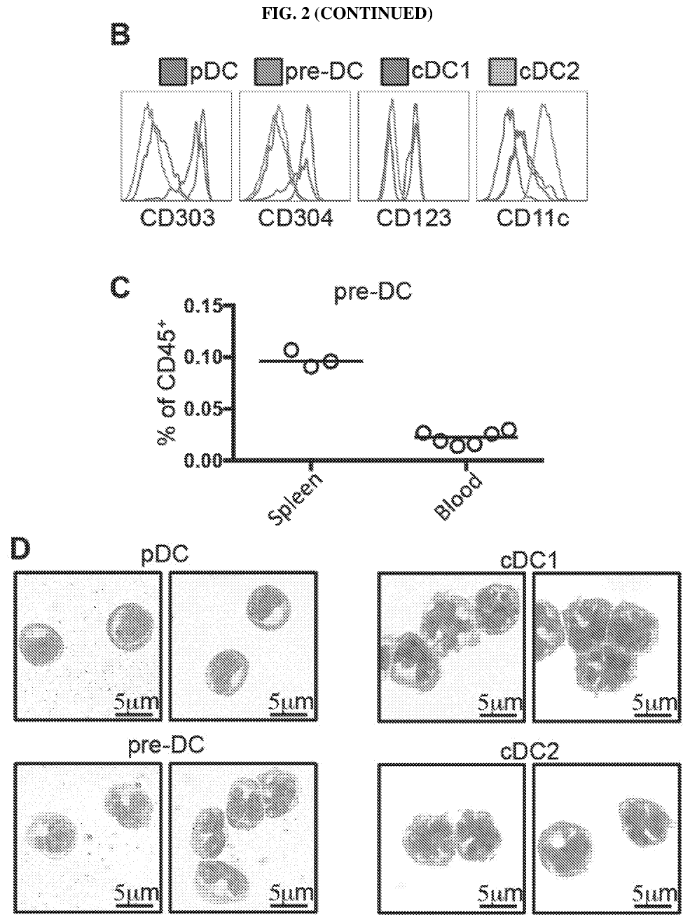

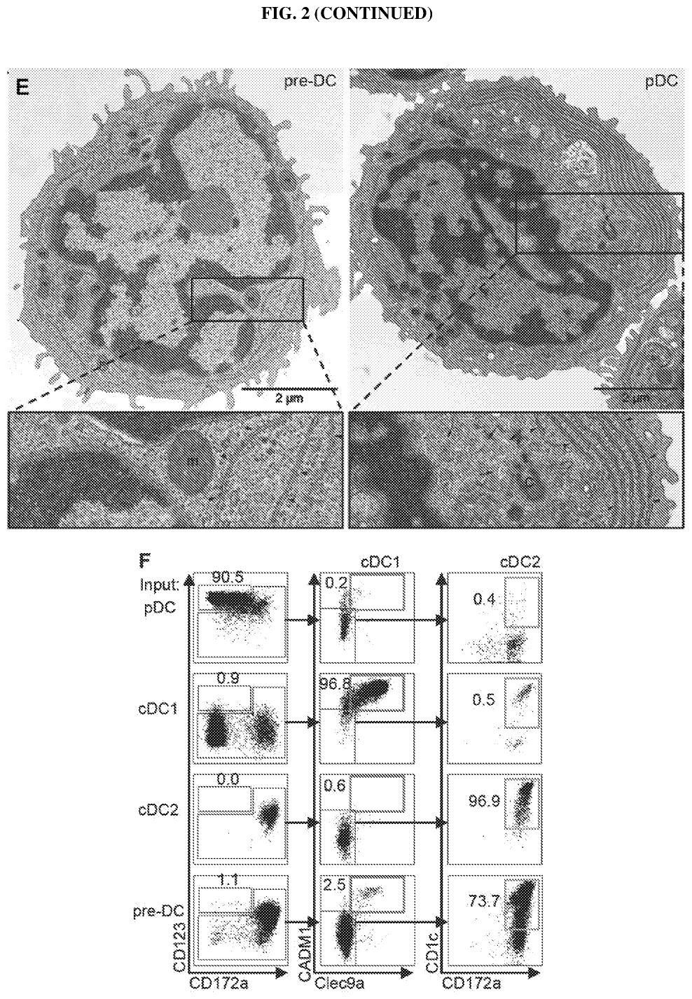

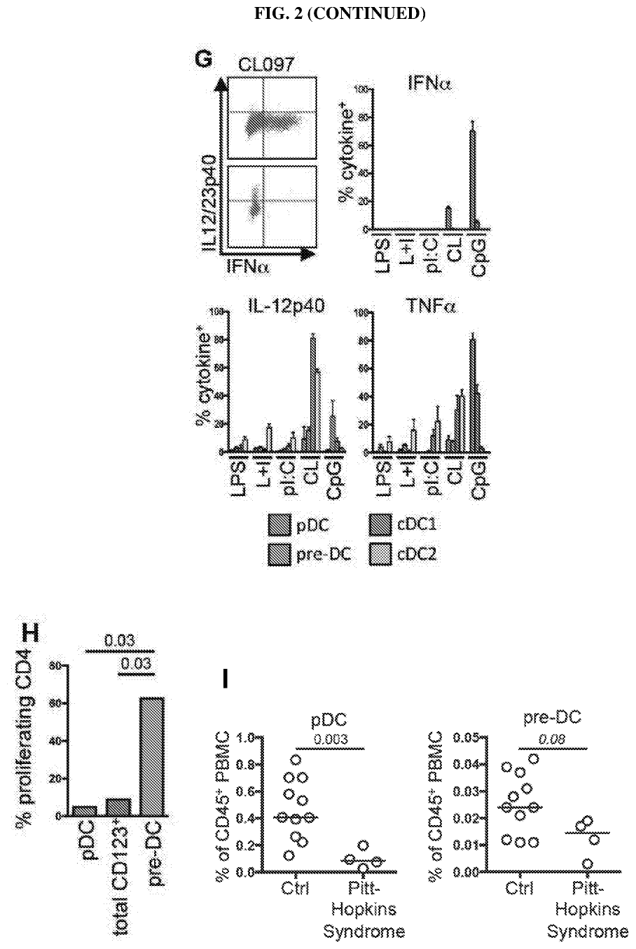

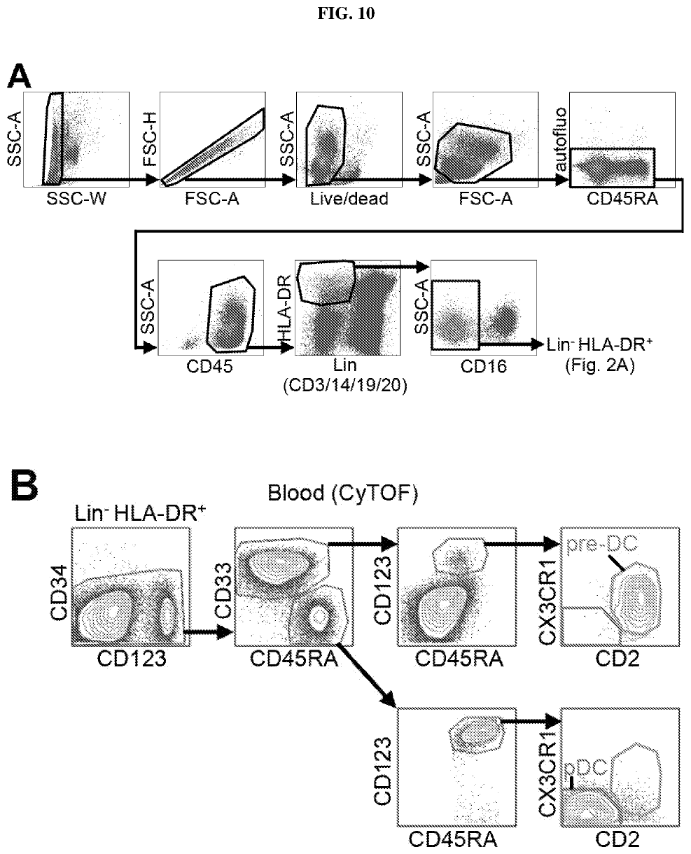

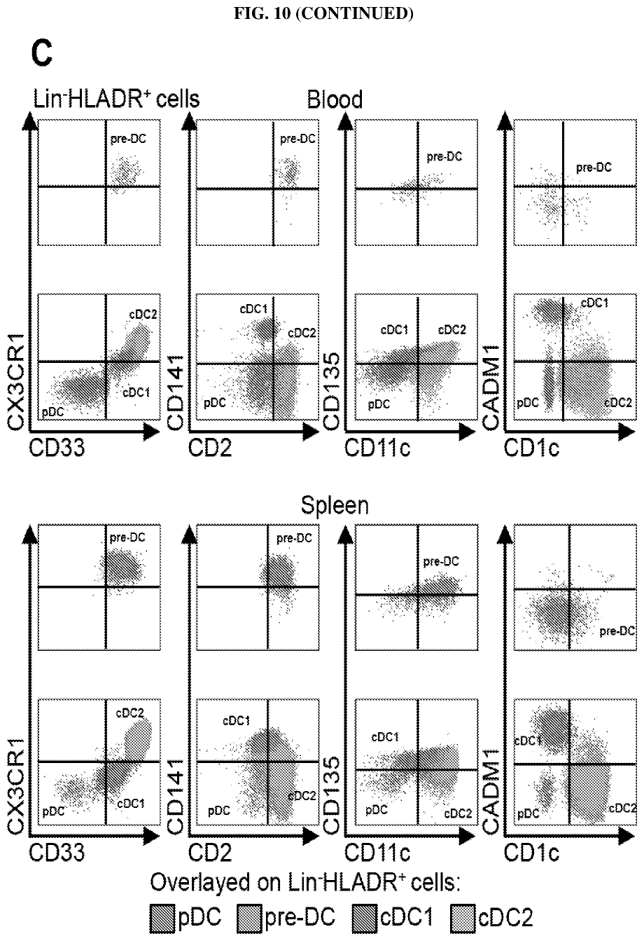

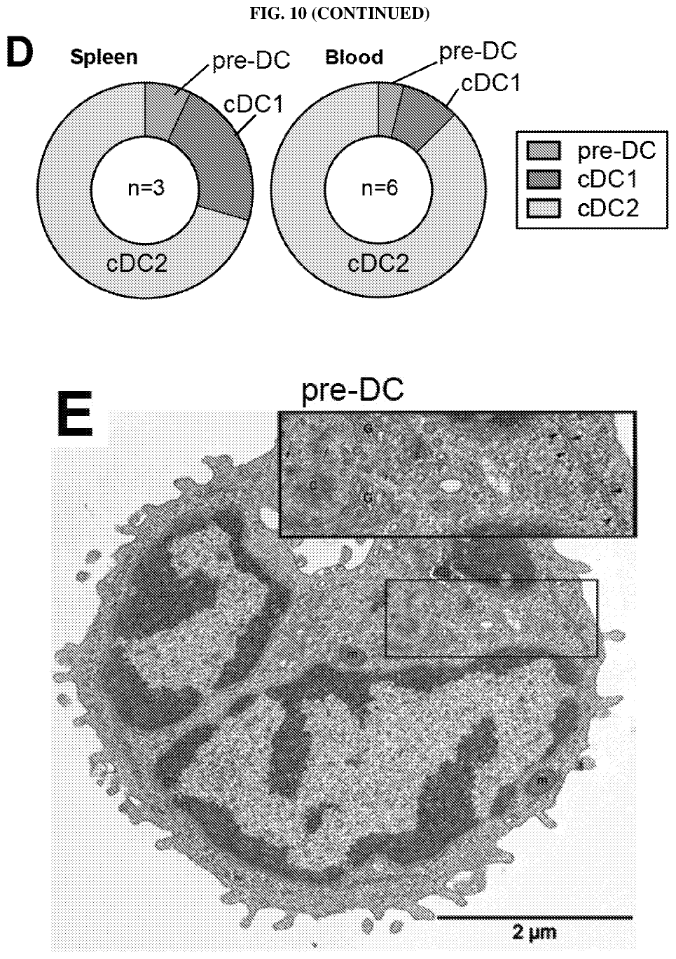

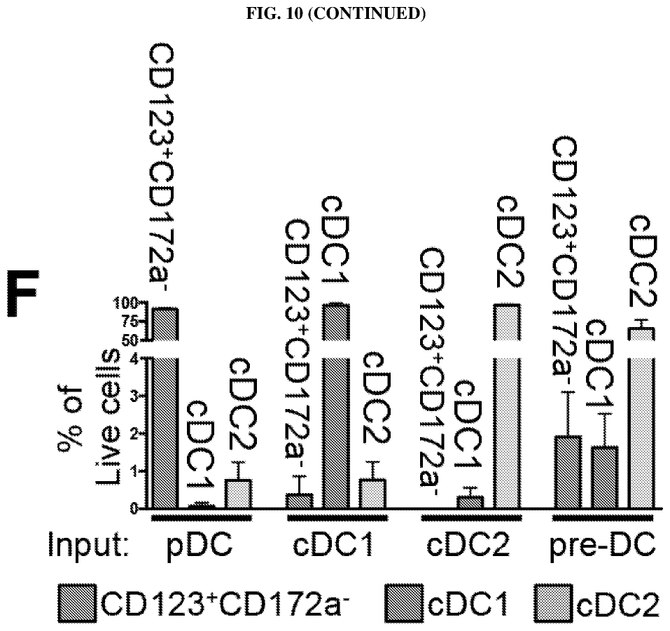

The CD123⁺CD33⁺ cell cluster within the Lin⁻HLA-DR⁺ fraction of the PBMC was analyzed by flow cytometry. Here, CD123⁺CD33⁻ pDC, CD45RA^{+/-}CD123⁻cDC1 and cDC2, and CD33⁺CD45RA⁺CD123⁺ putative pre-DC were identified (FIG. 2A, and FIG. 10A). The putative pre-DC expressed CX3CR1, CD2, CD303 and CD304, with low CD11c expression, whereas CD123⁺CD33⁻ pDC exhibited variable CD2 expression (FIGS. 2, A and B, and FIGS. 10, B and C).

The analysis was extended to immune cells from the spleen and a similar putative pre-DC population was identified, which was more abundant than in blood and expressed higher levels of CD11c (FIGS. 2, A and C, and FIG. 10D).

Both putative pre-DC populations in the blood and spleen expressed CD135 and intermediate levels of CD141 (FIG. 10C). Wright-Giemsa staining of putative pre-DC sorted from the blood revealed an indented nuclear pattern reminiscent of classical cDC, a region of perinuclear clearing, and a basophilic cytoplasm reminiscent of pDC (FIG. 2D).

At the ultra-structural level, putative pre-DC and pDC exhibited distinct features, despite their morphological similarities (FIG. 2E, and FIG. 10E): putative pre-DC possessed a thinner cytoplasm, homogeneously-distributed mitochondria (m), less rough endoplasmic reticulum (RER), an indented nuclear pattern, a large nucleus and limited cytosol, compared to pDC; pDC contained a smaller nucleus, abundant cytosol, packed mitochondria, well-developed and polarized cortical RER organized in parallel cisterna alongside numerous stacks of rough ER membranes, suggesting a developed secretory apparatus, in agreement with previously-published data (28).

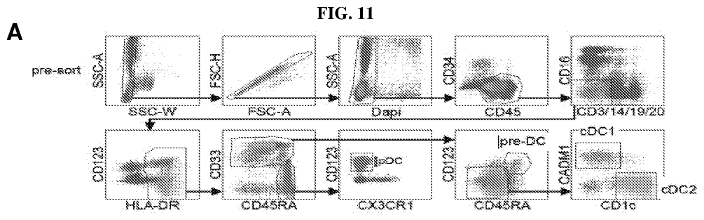

The differentiation capacity of pre-DC to that of cDC and pDC, through stromal culture in the presence of FLT3L, GMCSF and SCF was compared, as previously described (24). After 5 days, the pDC, cDC1 and cDC2 populations remained predominantly in their initial states, whereas the putative pre-DC population had differentiated into cDC1 and cDC2 in the known proportions found in vivo (29, 2, 30, 31) (FIG. 2F, FIG. 10F, and FIG. 11). Altogether, these data suggest that CD123⁺CD33⁺CD45RA⁺CX3CR1⁺CD2⁺ cells are circulating pre-DC with cDC differentiation potential.

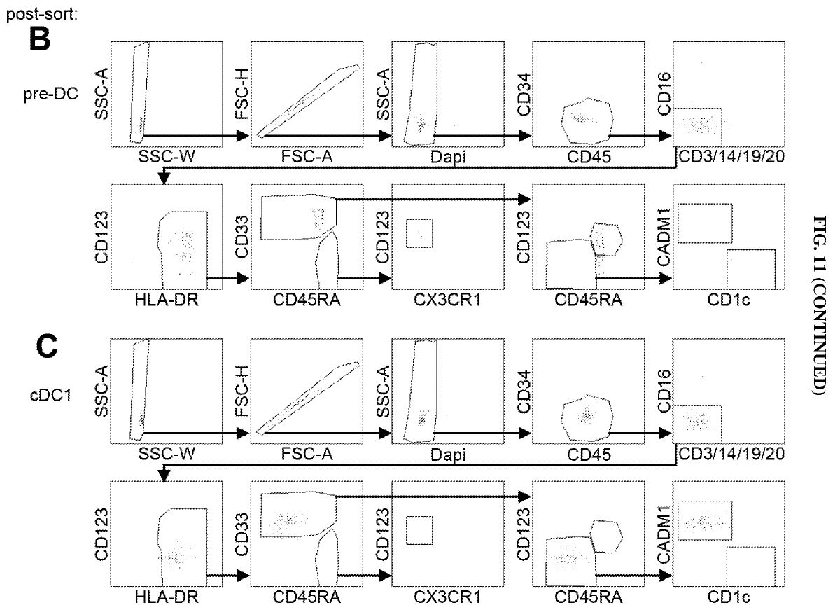

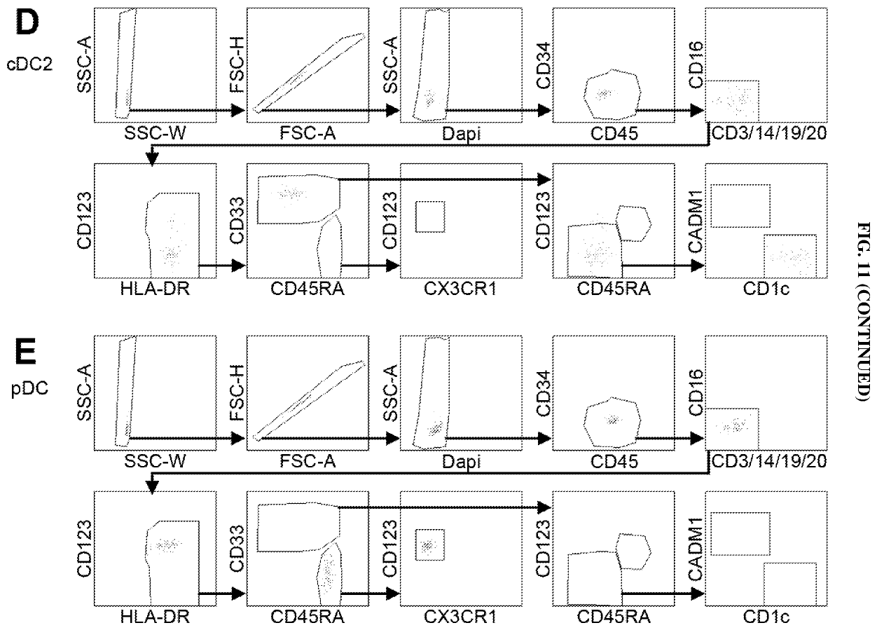

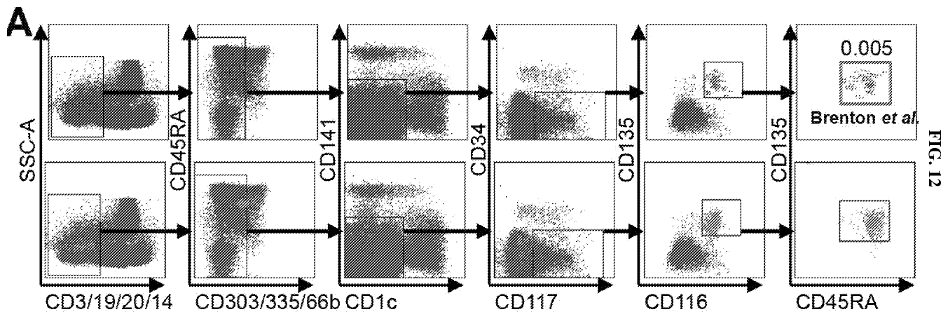

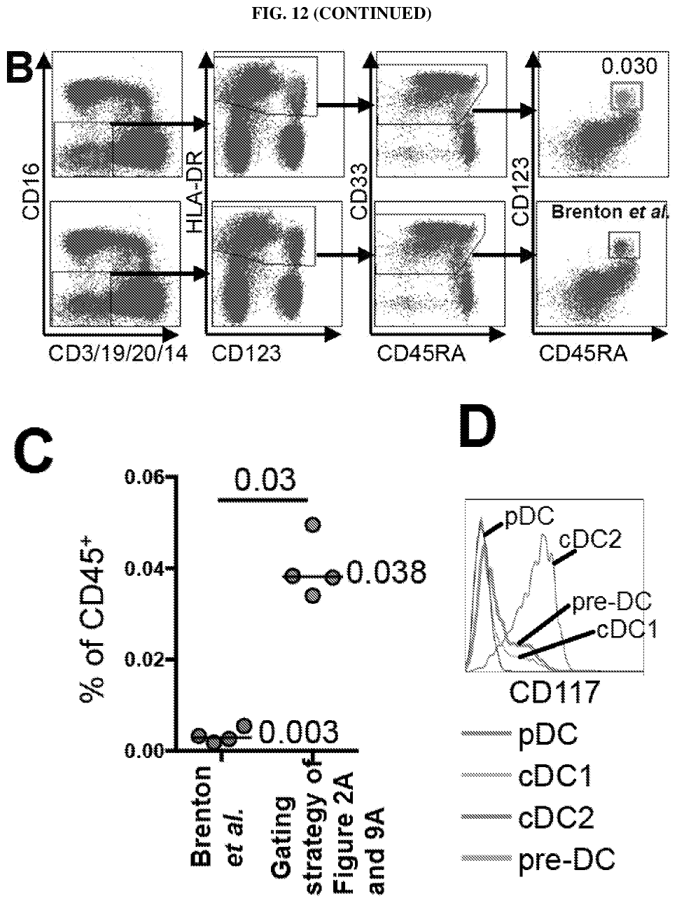

Breton and colleagues (32) recently reported a minor population of human pre-DC (highlighted in FIG. 12A), which shares a similar phenotype with the Lin⁻ CD123⁺CD33⁺CD45RA⁺ pre-DC defined here (FIGS. 12, A and B). The present results reveal that the pre-DC population in blood and spleen is markedly larger than the one identified within the minor CD303⁻CD141⁻CD117⁺ fraction considered previously (FIGS. 12, C and D).

Pre-DC are Functionally Distinct from pDC

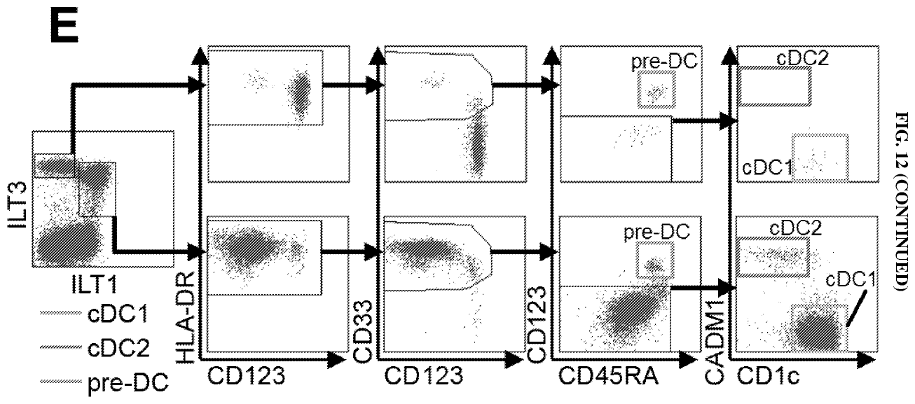

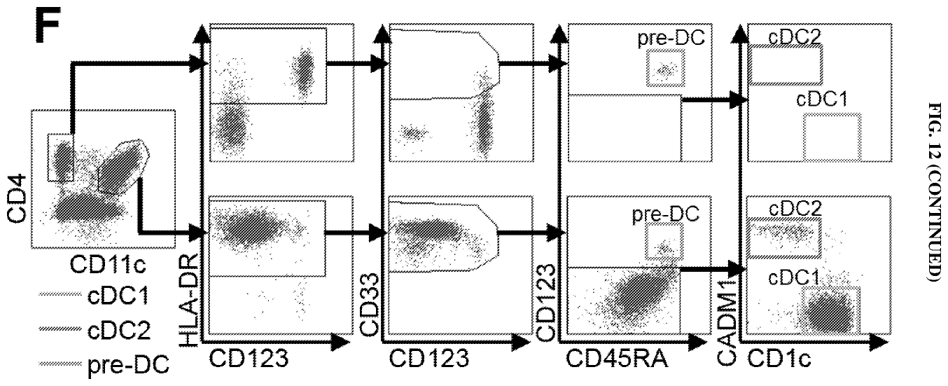

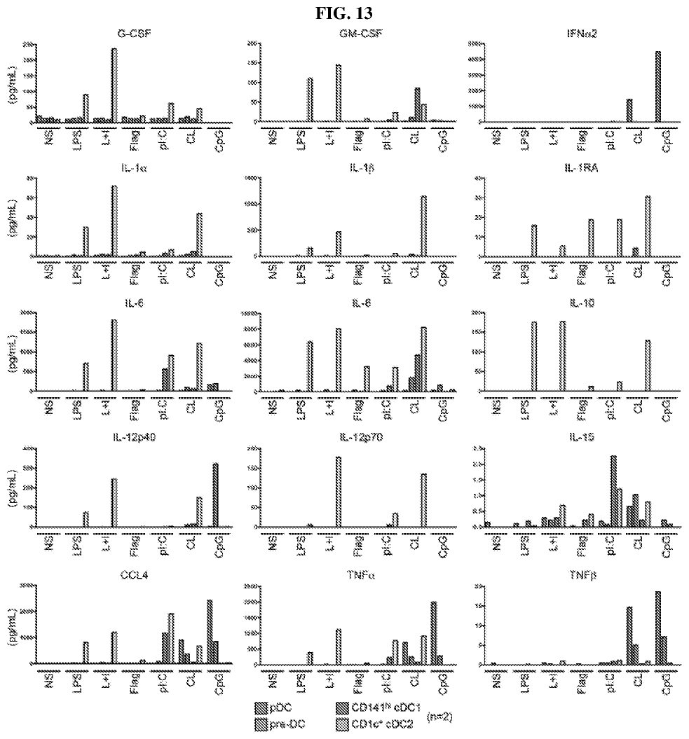

IFNα-secreting pDC can differentiate into cells resembling cDC when cultured with IL-3 and CD40L (33, 34), and have been considered DC precursors (34). However, when traditional ILT3⁺ILT1⁻ (33) or CD4⁺CD11c⁻ (34) pDC gating strategies were used, a “contaminating” CD123⁺CD33⁺CD45RA⁺ pre-DC sub-population in both groups was detected (FIGS. 12, E and F). This “contaminating” sub-population result raises the question on whether other properties of traditionally-classified “pDC populations” might be attributed to pre-DC. TLR7/8 (CL097) or TLR9 (CpG ODN 2216) stimulation of pure pDC cultures resulted in abundant secretion of IFNα, but not IL-12p40, whereas pre-DC readily secreted IL-12p40 but not IFN-α (FIG. 2G, and FIG. 13). Furthermore, while pDC were previously thought to induce proliferation of naïve CD4⁺ T cells (32, 35), here only the pre-DC sub-population was found to exhibit this attribute (FIG. 2H). Reports of potent allostimulatory capacity and IL-12p40 production by CD2⁺ pDC (35) might then be explained by CD2⁺ pre-DC “contamination” (36) (FIG. 14).

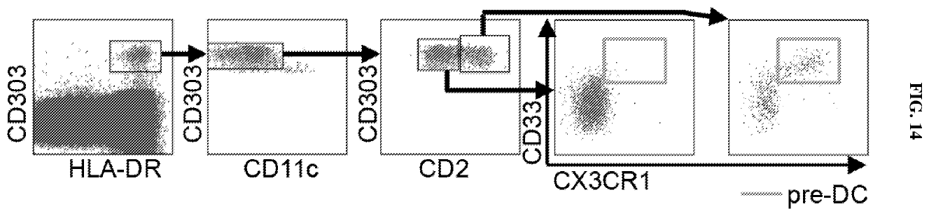

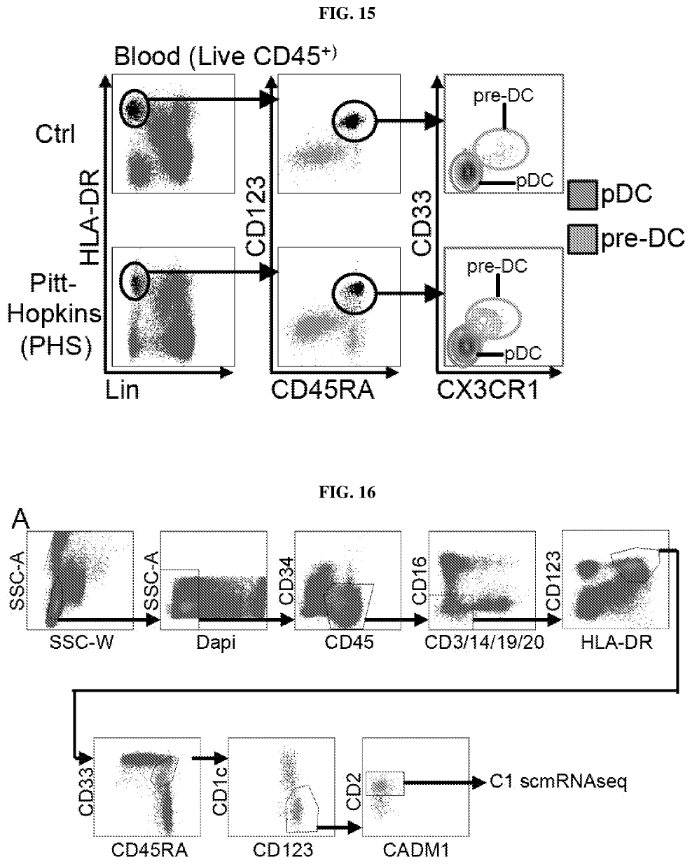

Pitt-Hopkins Syndrome (PHS) is characterized by abnormal craniofacial and neural development, severe mental retardation, and motor dysfunction, and is caused by haplo-insufficiency of TCF4, which encodes the E2-2 transcription factor—a central regulator of pDC development (37). Patients with PHS had a marked reduction in their blood pDC numbers compared to healthy individuals, but retained a population of pre-DC (FIG. 2I, and FIG. 15), which likely accounts for the unexpected CD45RA⁺CD123⁺CD303^{lo} cell population reported in these patients (57). Taken together, the present data indicate that, while pre-DC and pDC share some phenotypic features, they can be separated by their differential expression of several markers, including CD33, CX3CR1, CD2, CD5 and CD327. pDC are bona fide IFNα-producing cells, but the reported IL-12 production and CD4⁺ T-cell allostimulatory capacity of pDC can likely be attributed to “contaminating” pre-DC, which can give rise to both cDC1 and cDC2.

Identification and Characterization of Committed Pre-DC Subsets

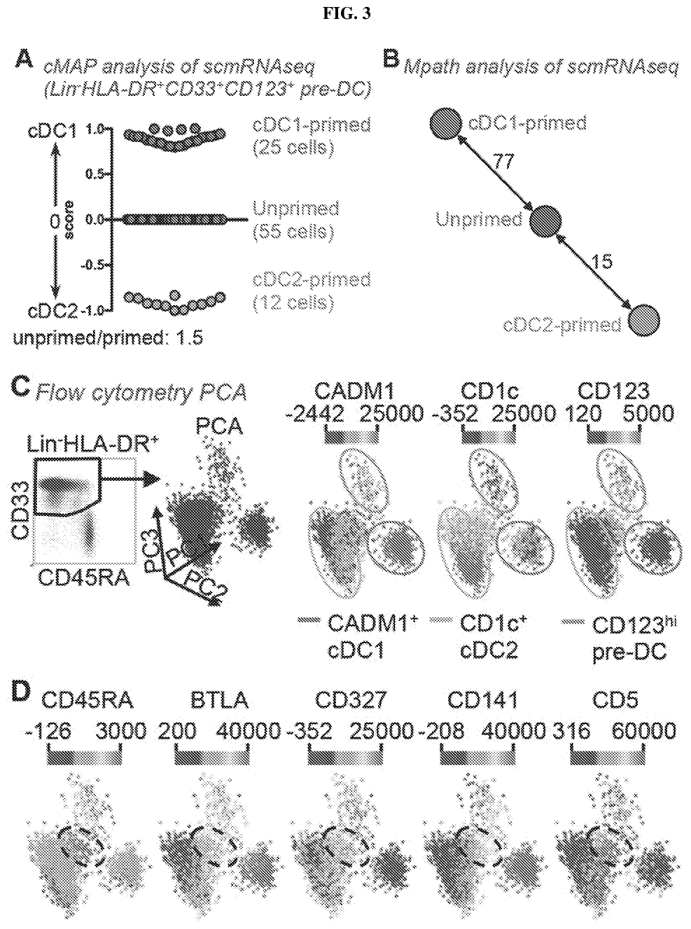

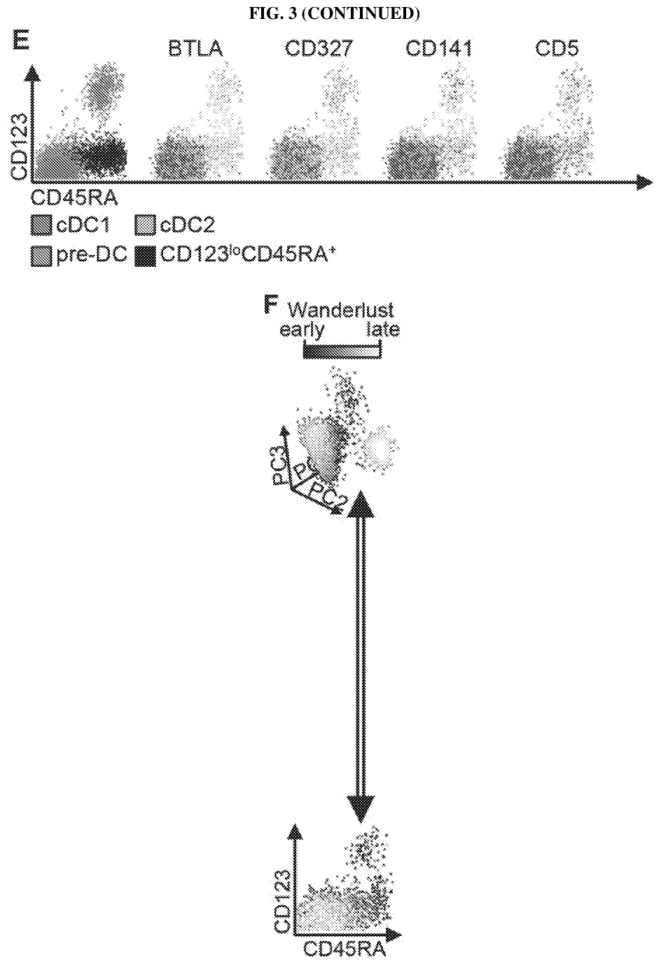

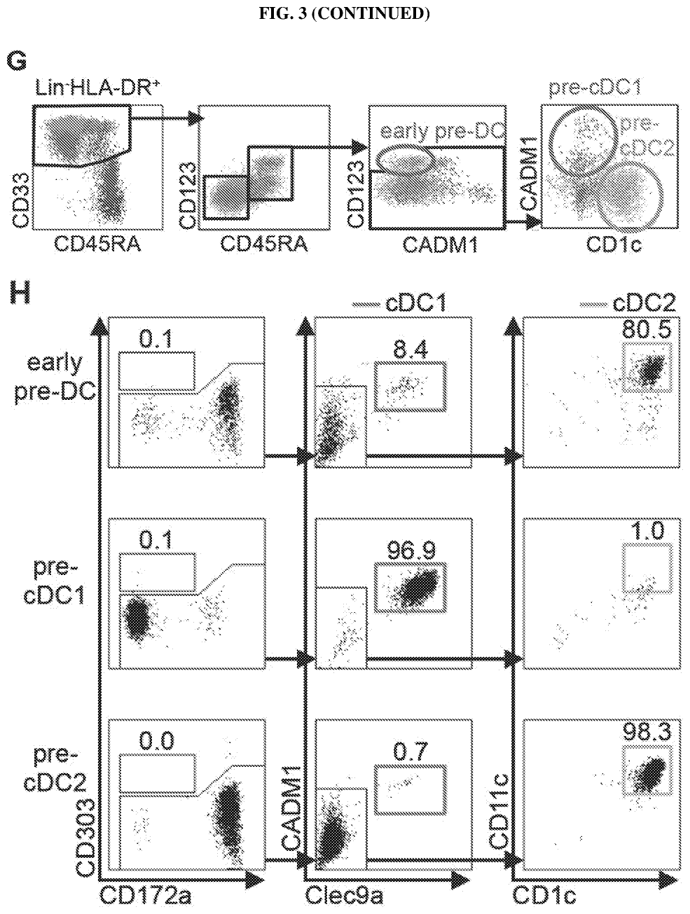

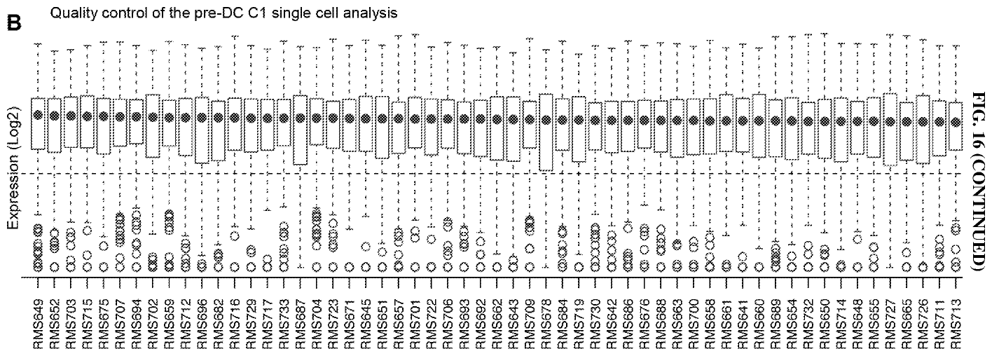

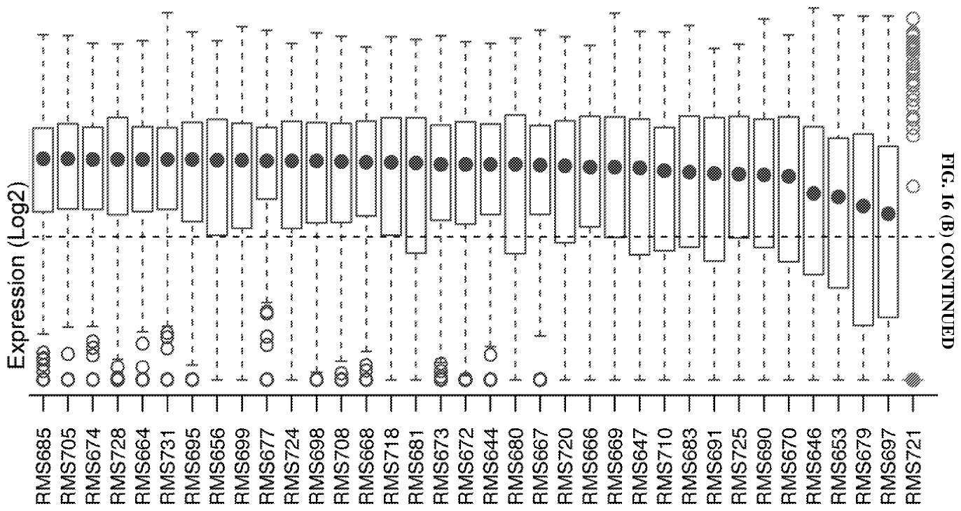

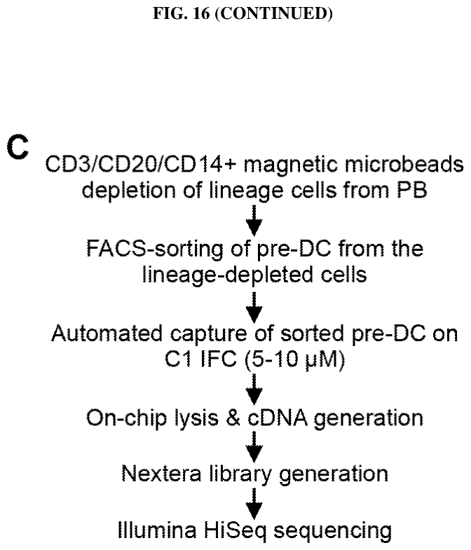

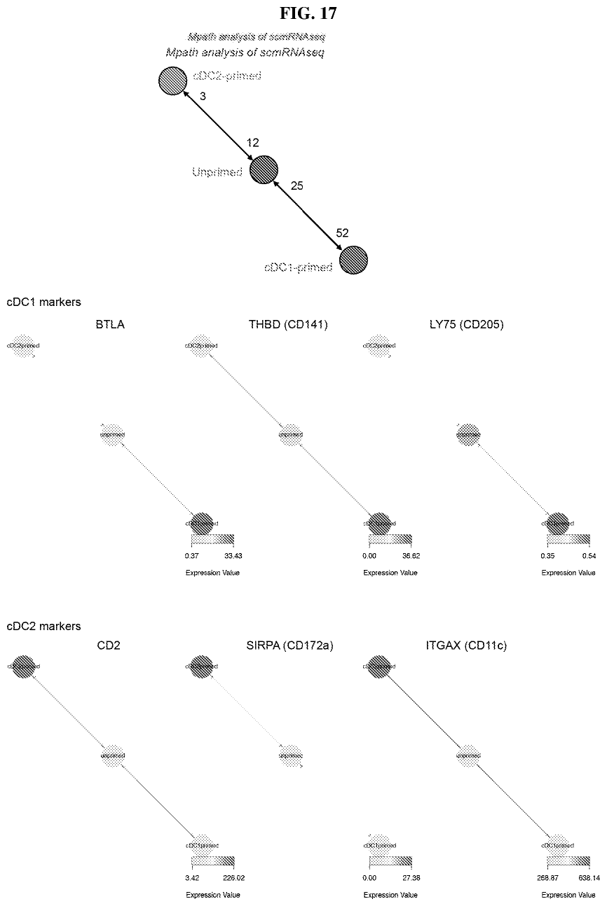

The murine pre-DC population contains both uncommitted and committed pre-cDC1 and pre-cDC2 precursors (38). Thus, microfluidic scmRNAseq was used to determine whether the same was true for human blood pre-DC, (FIG. 16A: sorting strategy, FIGS. 16, B and C: workflow and quality control, Table 4: number of expressed genes). The additional single cell gene expression data relative to the MARS-seq strategy used for FIG. 1, A to G (2.5 million reads/cell and an average of 4,742 genes detected per cell vs 60,000 reads/cell and an average of 749 genes detected per cell, respectively) was subjected to cMAP analysis, which calculated the degree of enrichment for cDC1 or cDC2 signature gene transcripts (2) for each single cell (FIG. 3A). Among the 92 analyzed pre-DC, 25 cells exhibited enrichment for cDC1 gene expression signatures, 12 cells for cDC2 gene expression signatures, and 55 cells showed no transcriptional similarity to either cDC subset. Further Mpath analysis showed that these 55 “unprimed” pre-DC were developmentally related to cDC1-primed and cDC2-primed pre-DC, and thus their patterns of gene expression fell between the cDC1 and cDC2 signature scores by cMAP (FIG. 3B, and FIG. 17). These data suggest that the human pre-DC population contains cells exhibiting transcriptomic priming towards cDC1 and cDC2 lineages, as observed in mice (38).

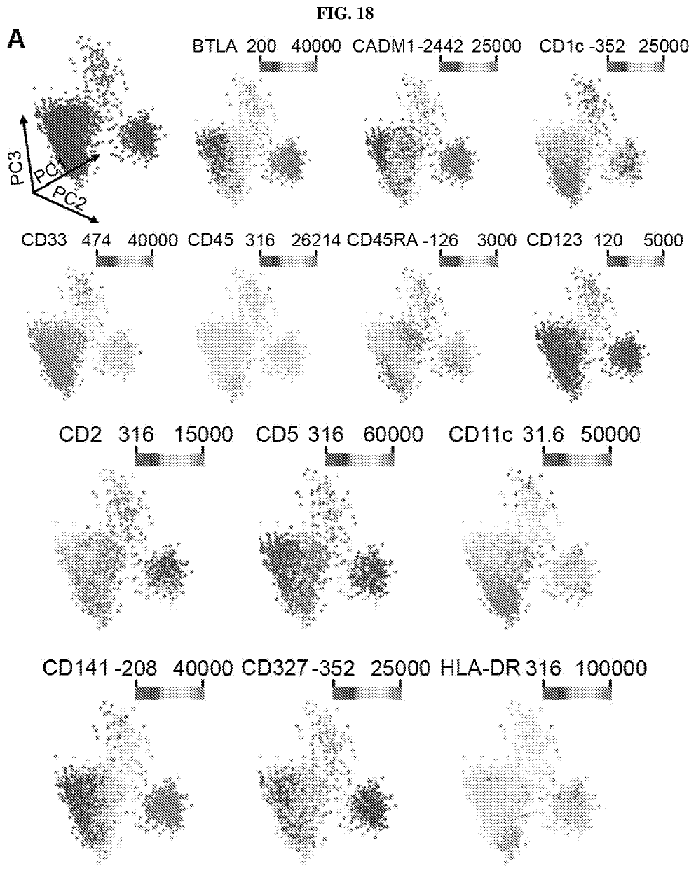

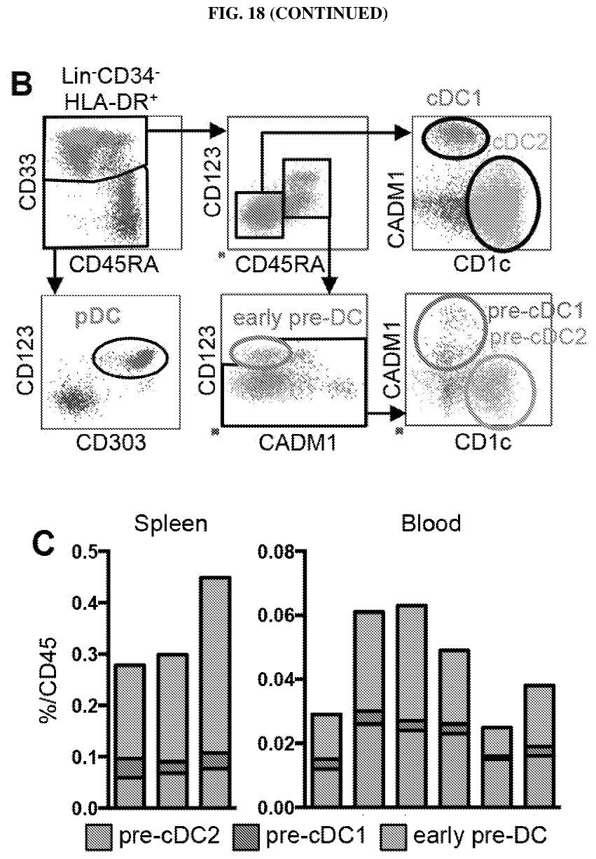

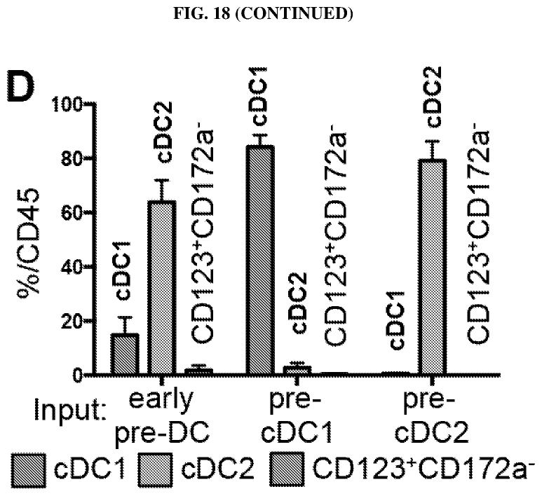

This heterogeneity within the pre-DC population by flow cytometry were further subjected to identification using either pre-DC-specific markers (CD45RA, CD327, CD5) or markers expressed more intensely by pre-DC compared to cDC2 (BTLA, CD141). 3D-PCA analysis of the Lin⁻HLA-DR⁺CD33⁺ population (containing both differentiated cDC and pre-DC) identified three major cell clusters: CADM1⁺cDC1, CD1c⁺cDC2 and CD123⁺ pre-DC (FIG. 3C, and FIG. 18A). Interestingly, while cells located at the intersection of these three clusters (FIG. 3D) expressed lower levels of CD123 than pre-DC, but higher levels than differentiated cDC (FIG. 3C), they also expressed high levels of pre-DC markers (FIG. 3D, and FIG. 18A). It is possible that these CD45RA⁺CD123^{lo} cells might be committed pre-DC that are differentiating into either cDC1 or cDC2 (FIG. 3E). The Wanderlust algorithm (39), which orders cells into a constructed trajectory according to their maturity, confirmed the developmental relationship between pre-DC (early events), CD45RA⁺CD123^{lo} cells (intermediate events) and mature cDC (late events) (FIG. 3F). Flow cytometry of PBMC identified CD123⁺CADM1⁻CD1c⁻ putative uncommitted pre-DC, alongside putative CADM1⁺CD1c⁻ pre-cDC1 and CADM1⁻CD1c⁺ pre-cDC2 within the remaining CD45RA⁺ cells (FIG. 3G, and FIG. 18B). These three populations were also present, and more abundant, in the spleen (FIG. 18C). Importantly, in vitro culture of pre-DC subsets sorted from PBMC did not give rise to any CD303⁺ cells (which would be either undifferentiated pre-DC or differentiated pDC), whereas early pre-DC gave rise to both cDC subsets, and pre-cDC1 and pre-cDC2 differentiated exclusively into cDC1 and cDC2 subsets, respectively (FIG. 3H, FIG. 18D, and FIG. 19).

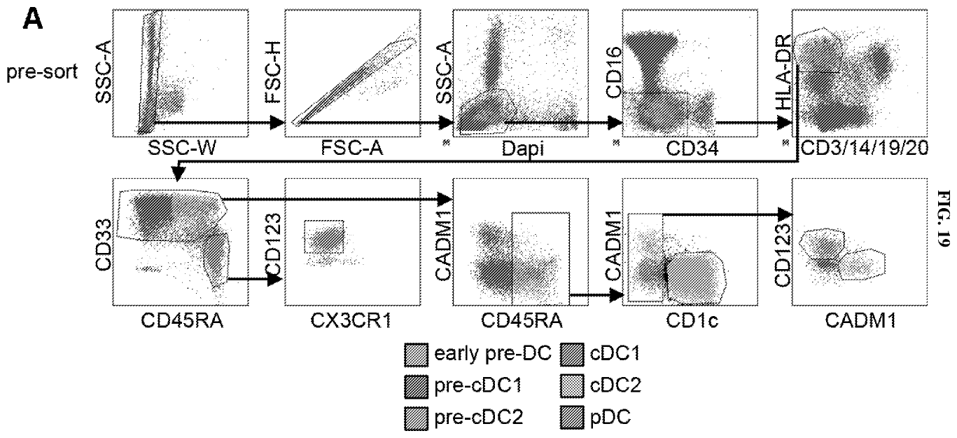

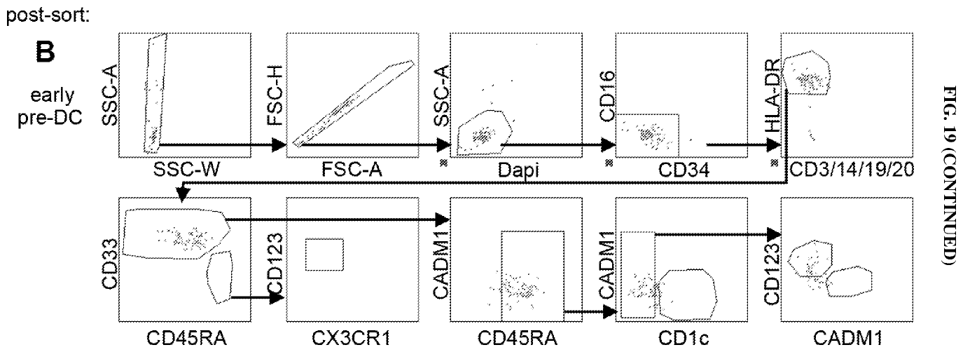

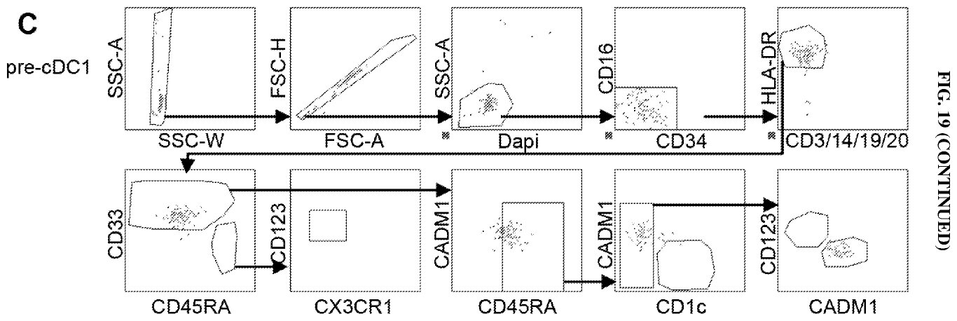

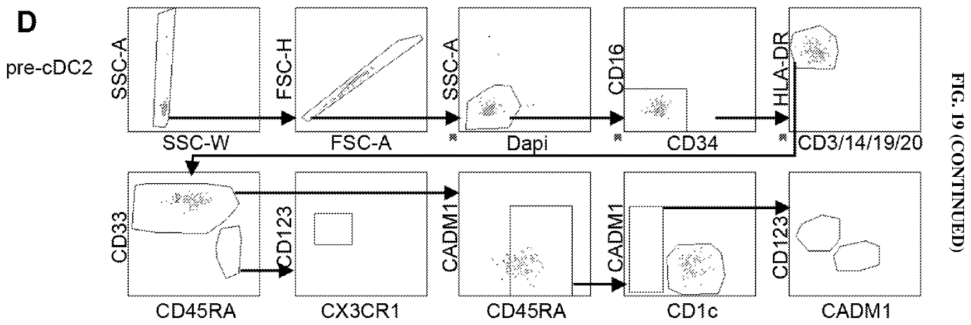

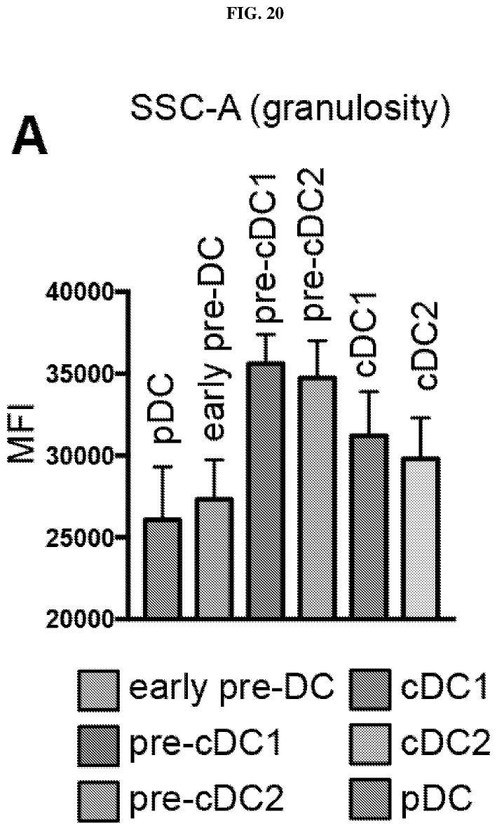

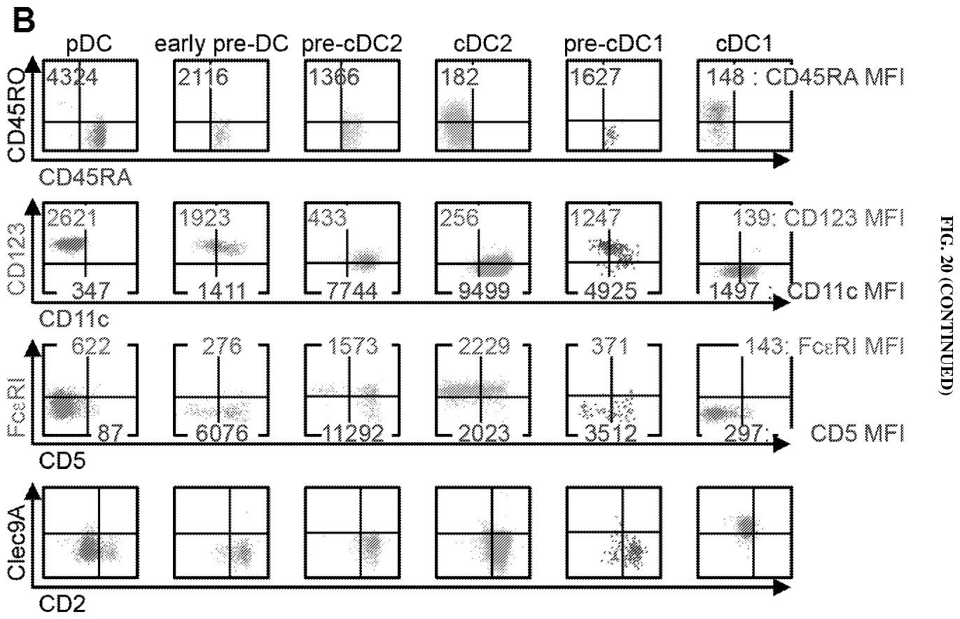

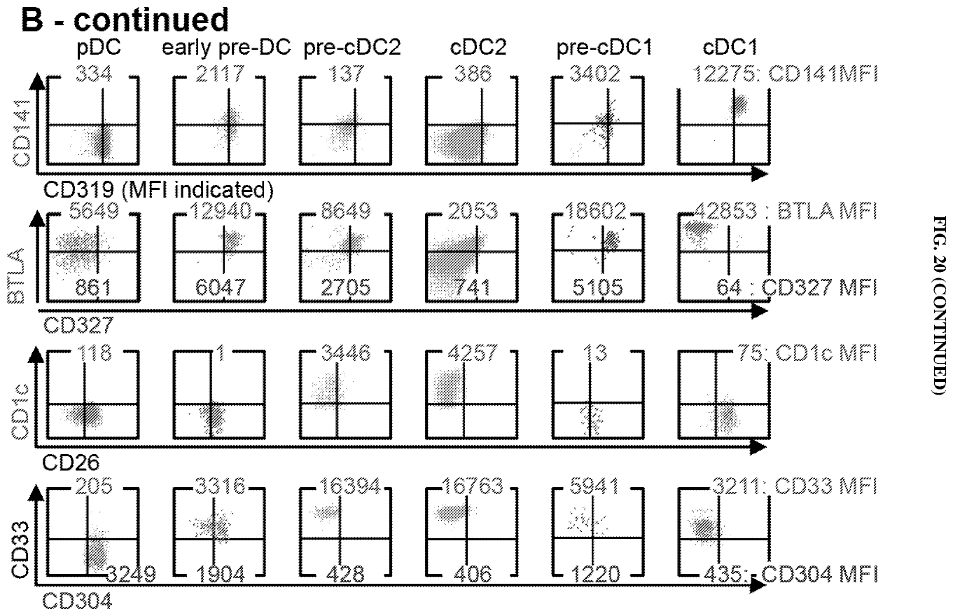

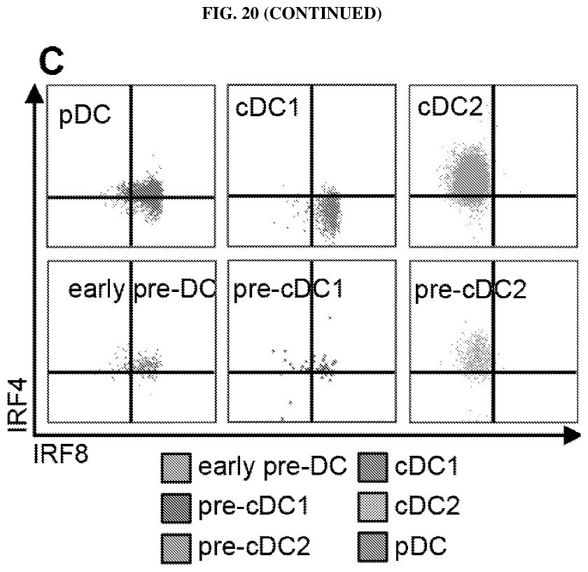

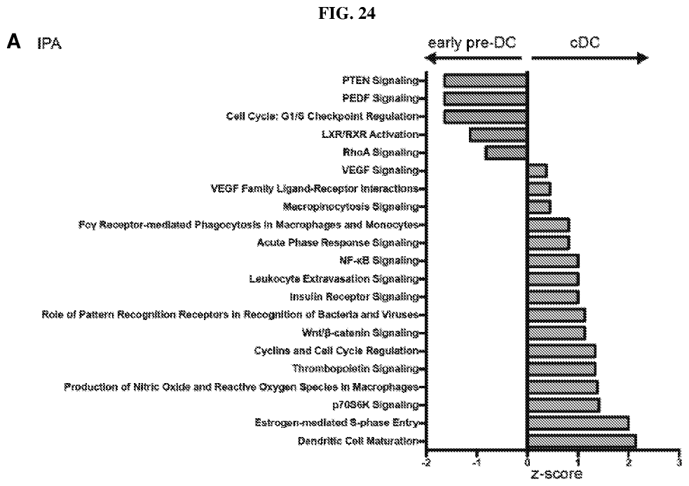

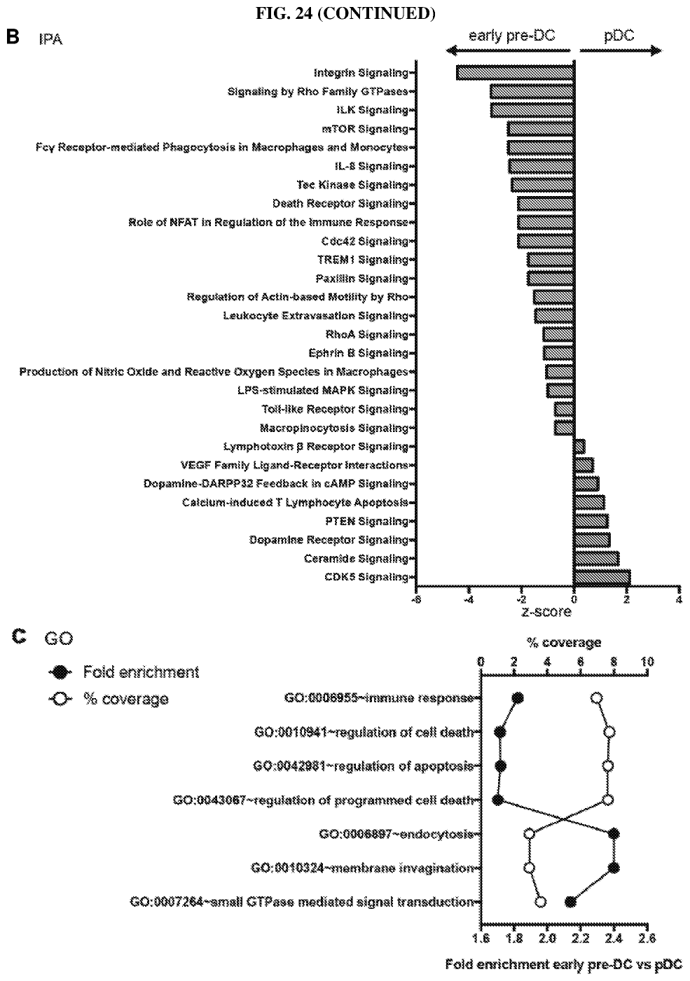

Scanning electron microscopy confirmed that early pre-DC are larger and rougher in appearance than pDC, and that committed pre-DC subsets closely resemble their mature cDC counterparts (FIG. 3I, and FIG. 20A). Phenotyping of blood pre-DC by flow cytometry (FIG. 24B) identified patterns of transitional marker expression throughout the development of early pre-DC towards pre-cDC1/2 and differentiated cDC1/2. Specifically, CD45RO and CD33 were acquired in parallel with the loss of CD45RA; CD5, CD123, CD304 and CD327 were expressed abundantly by early pre-DC, intermediately by pre-cDC1 and pre-cDC2, and rarely if at all by mature cDC and pDC; FccRI and CD1c were acquired as early pre-DC commit towards the cDC2 lineage, concurrent with the loss of BTLA and CD319 expression; early pre-DC had an intermediate expression of CD141 that dropped along cDC2 differentiation but was increasingly expressed during commitment towards cDC1, with a few pre-cDC1 already starting to express Clec9A; and IRF8 and IRF4—transcription factors regulating cDC lineage development (40, 41)—were expressed by early pre-DC and pre-cDC1, while pre-cDC2 maintained only IRF4 expression (FIG. 20C).

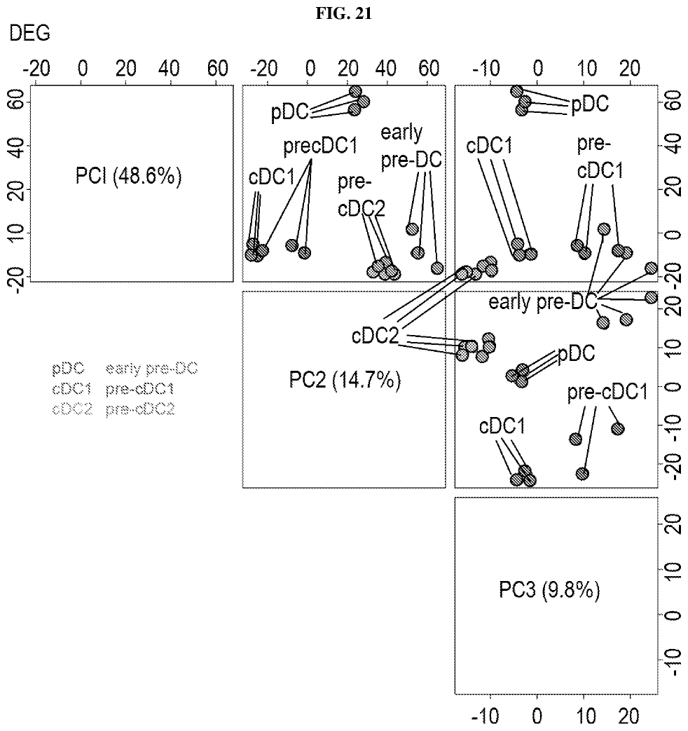

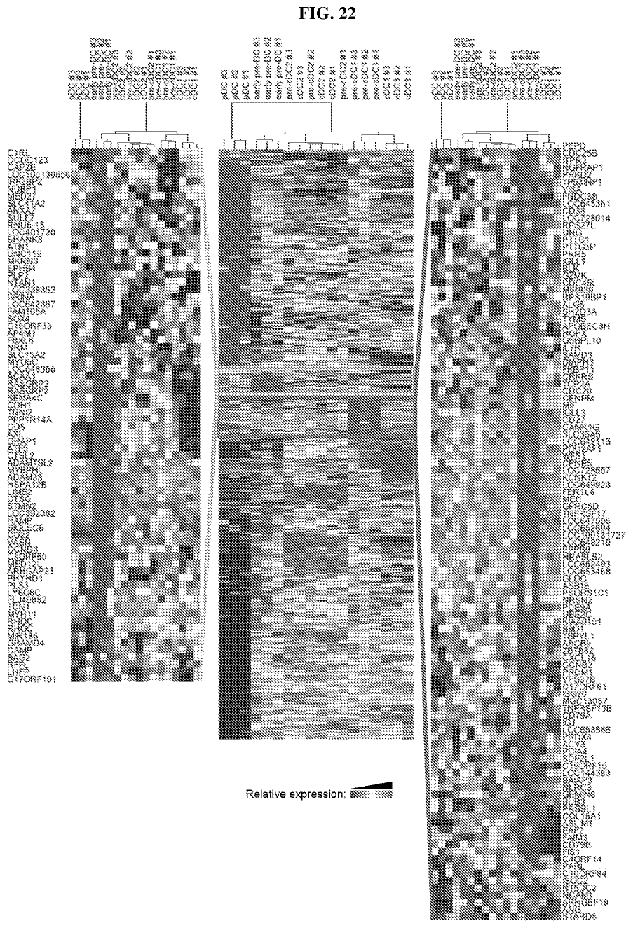

Pre-DC and DC subsets were next sorted from blood and microarray analyses were performed to define their entire transcriptome. 3D-PCA analysis of the microarray data showed that pDC were clearly separated from other pre-DC and DC subsets along the horizontal PC1 axis (FIG. 4A, and FIG. 21). The combination of the PC2 and PC3 axes indicated that pre-cDC1 occupied a position between early pre-DC and cDC1 and, although cDC2 and pre-cDC2 exhibited similar transcriptomes, pre-cDC2 were positioned between cDC2 and early pre-DC along the PC3 axis (FIG. 4A). Hierarchical clustering of differentially-expressed genes (DEG) confirmed the similarities between committed pre-DC and their corresponding mature subset (FIG. 22). The greatest number of DEG was between early pre-DC and pDC (1249 genes) among which CD86, CD2, CD22, CD5, ITGAX (CD11c), CD33, CLEC10A, SIGLEC6 (CD327), THBD, CLEC12A, KLF4 and ZBTB46 were more highly expressed by early pre-DC, while pDC showed higher expression of CD68, CLEC4C, TCF4, PACSIN1, IRF7 and TLR7 (FIG. 4B). An evolution in the gene expression pattern was evident from early pre-DC, to pre-cDC1 and then cDC1 (FIG. 4C), whereas pre-cDC2 were similar to cDC2 (FIG. 4D, and FIG. 22). The union of DEGs comparing pre-cDC1 versus early pre-DC and cDC1 versus pre-cDC1 has 62 genes in common with the union of DEGs from comparing pre-cDC2 versus early pre-DC and cDC2 versus pre-cDC2. These 62 common genes include the transcription factors BATF3, ID2 and TCF4 (E2-2), and the pre-DC markers CLEC4C (CD303), SIGLEC6 (CD327), and IL3RA (CD123) (FIG. 4E, FIG. 23 and Table 5). The progressive reduction in transcript abundance of SIGLEC6 (CD327), CD22 and AXL during early pre-DC to cDC differentiation was also mirrored at the protein level (FIG. 4F). Key transcription factors involved in the differentiation and/or maturation of DC subsets showed a progressive change in their expression along the differentiation path from pre-DC to mature cDC (FIG. 4G). Finally, pathway analyses revealed that pre-DC exhibited an enrichment of cDC functions relative to pDC, and were maintained in a relatively immature state compared to mature cDC (FIG. 24).

Committed Pre-DC Subsets are Functional

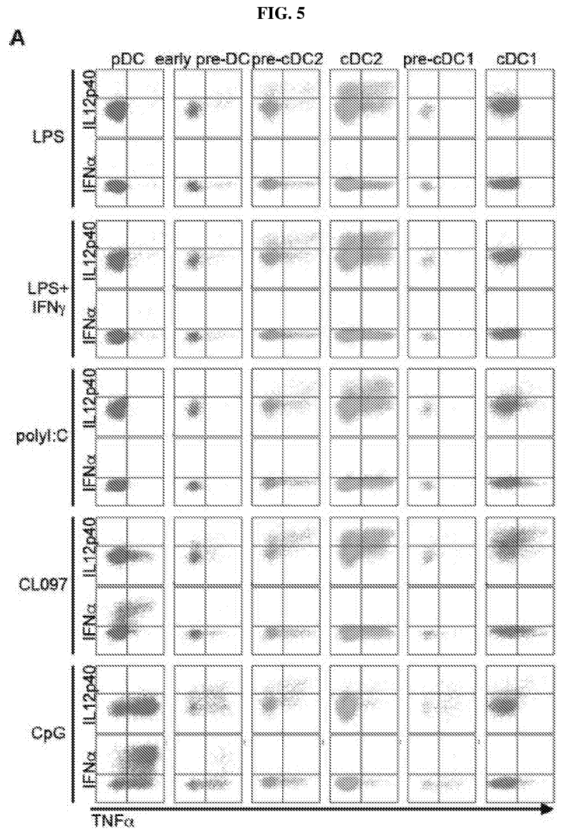

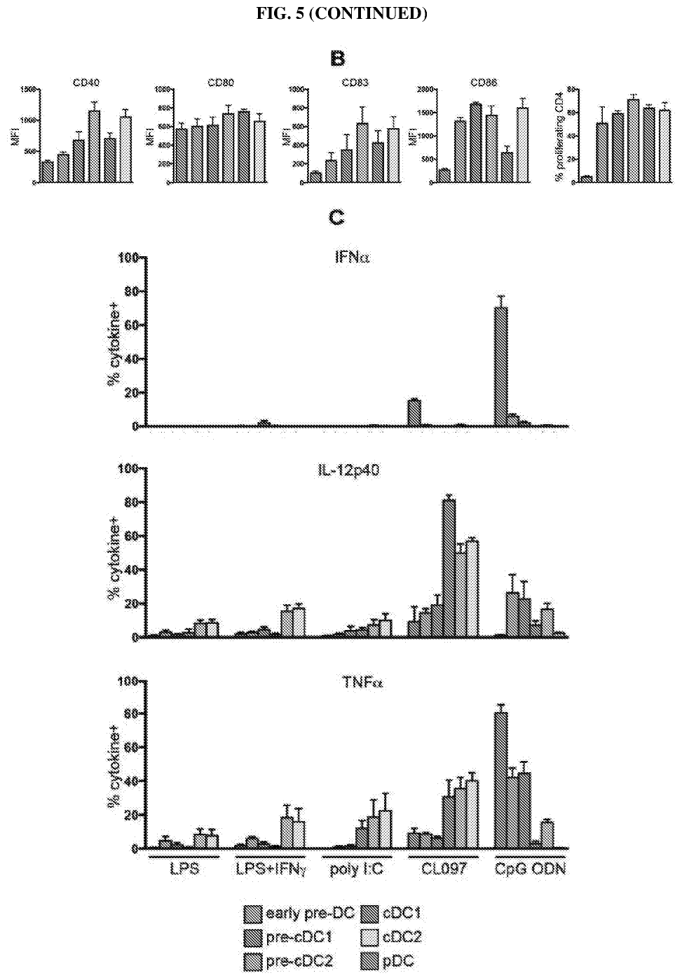

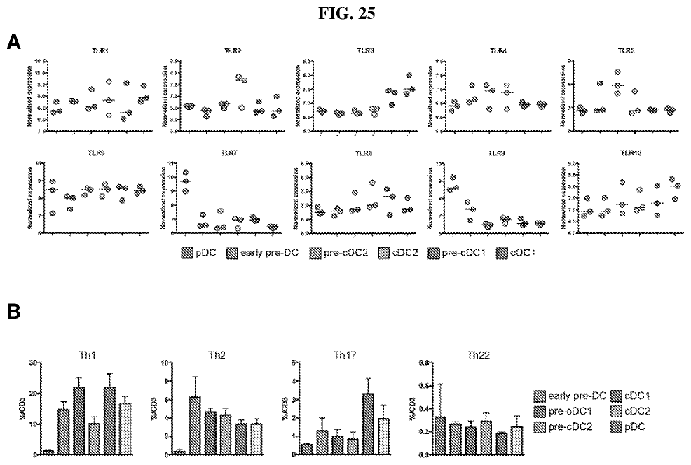

The present invention then investigated to what extent the functional specializations of DC (42, 43) were acquired at the precursor level by stimulating PBMC with TLR agonists and measuring their cytokine production (FIG. 5A). Pre-DC produced significantly more TNF-α and IL-12p40 when exposed to CpG ODN 2216 (TLR9 agonist), than to either LPS (TLR4 agonist) or polyI:C (TLR3 agonist) (p=0.03, Mann-Witney test). It was confirmed that pDC were uniquely capable of robust IFN-α production in response to CL097 and CpG ODN 2216. CpG ODN 2216 stimulation also triggered IL-12p40 and TNF-α production by early pre-DC, pre-cDC1, and to a lesser extent pre-cDC2. Although TLR9 transcripts were detected only in early pre-DC (FIG. 25A), these data indicate that, contrary to differentiated cDC1 and cDC2, pre-cDC1 and pre-cDC2 do express functional TLR9 and hence can be activated using TLR9 agonists. Interestingly, while pre-cDC2 resembled cDC2 at the gene expression level, their responsiveness to TLR ligands was intermediate between that of early pre-DC and cDC2. Pre-DC subsets also expressed T-cell co-stimulatory molecules (FIG. 5B) and induced proliferation and polarization of naïve CD4⁺ T cells to a similar level as did mature cDC (FIG. 5C, and FIG. 25B).

Unsupervised Mapping of DC Ontogeny

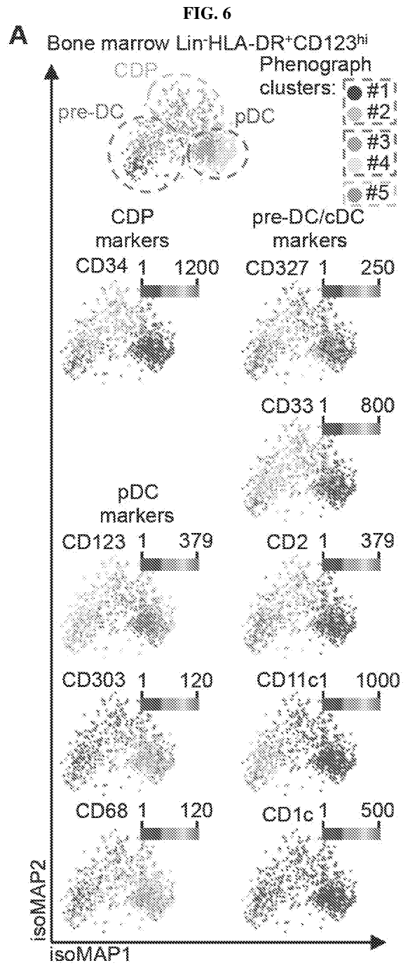

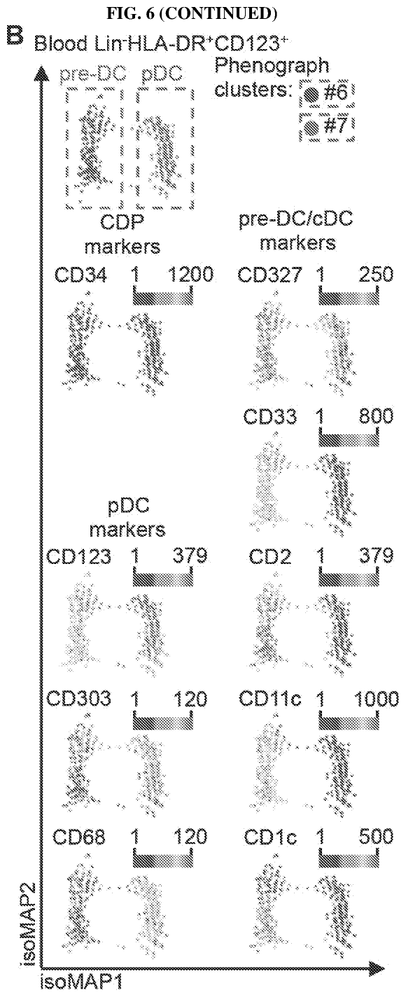

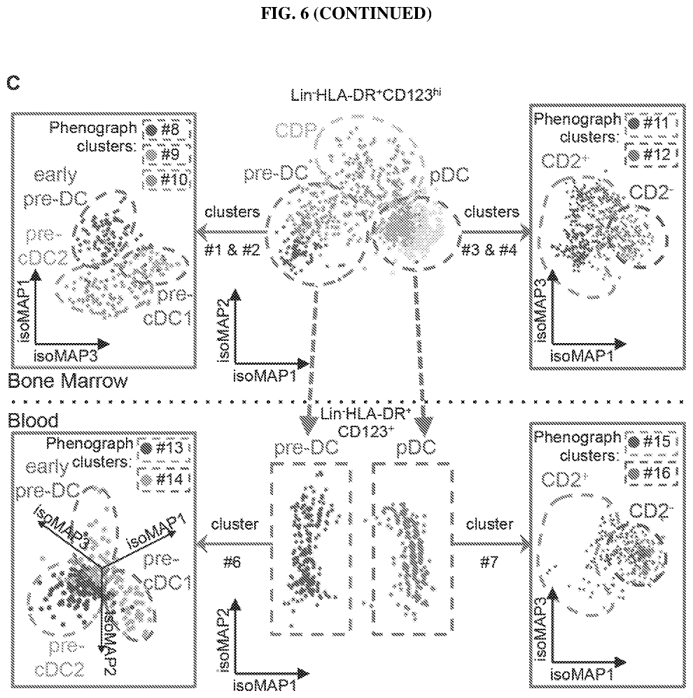

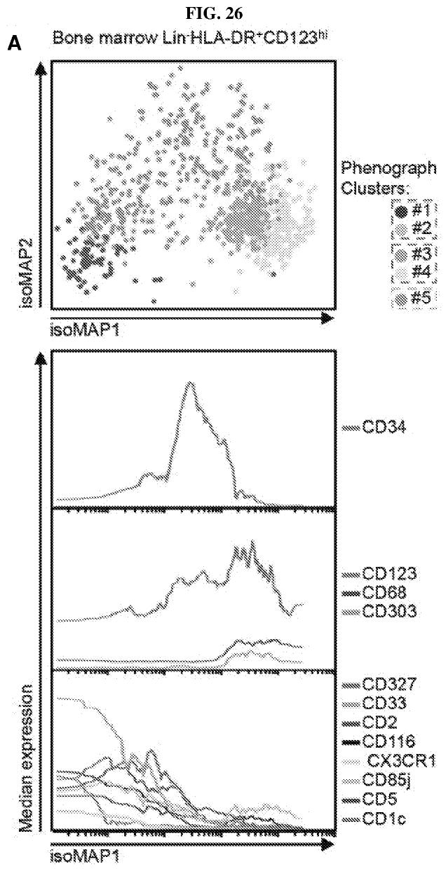

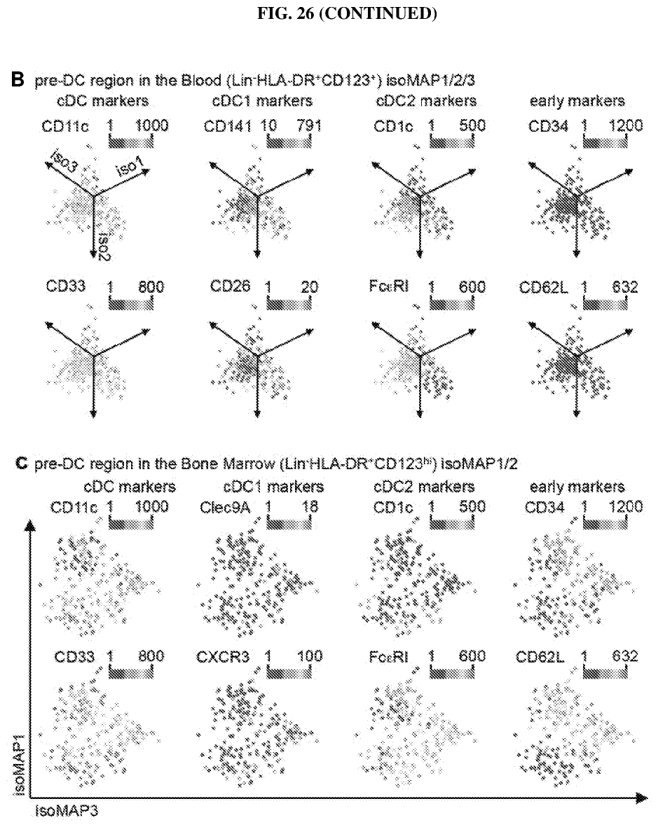

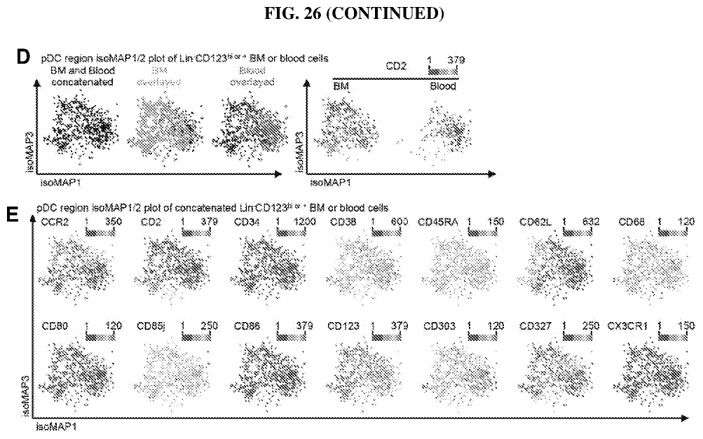

To understand the relatedness of the cell subsets, an unsupervised isoMAP analysis (20) was performed of human BM cells, obtained from CyTOF analysis, for non-linear dimensionality reduction (FIG. 6A, and FIG. 26A). This analysis focused on the Lin⁻CD123^{hi} fraction and identified CD123^{hi}CD34⁺CDP (phenograph cluster #5), from which branched CD34⁻CD123⁺CD327⁺CD33⁺ pre-DC (clusters #1 and #2) and CD34⁻D123⁺CD303⁺CD68⁺ pDC (clusters #3 and #4) which both progressively acquired their respective phenotypes. Cells in the pre-DC branch increasingly expressed CD2, CD11c, CD116 and, at a later stage, CD1c. IsoMAP analysis of Lin⁻CD123⁺ cells in the peripheral blood identified two parallel lineages, corresponding to pre-DC and pDC, in which a CDP population was not detected (FIG. 6B). IsoMAP and phenograph analysis of pre-DC extracted from the isoMAP analysis of FIG. 6A (BM, clusters #1 and #2) and FIG. 6B (blood, cluster #6) revealed the three distinct pre-DC subsets (FIG. 6C) as defined by their unique marker expression patterns (FIGS. 26, B and C).

In summary, the developmental stages of DC from the BM to the peripheral blood through CyTOF were traced, which shows that the CDP population in the BM bifurcates into two pathways, developing into either pre-DC or pDC in the blood (FIG. 6, A to C). This pre-DC population is heterogeneous and exists as distinct subsets detectable in both the blood and BM (FIG. 6C, and FIGS. 26, B and C). Furthermore, an intriguing heterogeneity in blood and BM pDC was uncovered, which warrants further investigation (FIG. 6C, and FIGS. 26, D and E).

Validation of Down Sampling Threshold for Normalization of MARS-Seq Single Cell Transcriptome Data

High variance in terms of quality of single-cell transcriptomes is expected in a single-cell RNA sequencing experiment due to the low quantity of RNA input material. This caveat necessitates stringent quality control in order to avoid a bias introduced by low quality single-cell transcriptomes. In single-cell transcriptomics it is, therefore, common practice to remove low quality transcriptomes to ensure an unbiased and biologically meaningful analysis (44, 45). Different strategies have been used to filter out low quality cells, including an empirically determined cutoff for cell filtering (45), a down sampling strategy to normalize and filter low quality cells (3), and various filtering cutoffs from 600 UMIs/cell or 400 UMIs/cells (3), <500 molecule counts per cell (46) and <200 UMIs/cell (47). A mathematically determined cut-off was not reported in any of these studies. As these previous studies were performed on murine cells, and quality filters in single-cell data have to be established within the respective dataset, the present approach had adapted the filtering strategy to human cells. To determine the quality threshold for the present dataset, several diagnostics were used to estimate the optimal cutoff for down sampling of molecule counts. Firstly, the cumulative distribution of molecule counts were visualized, where cells on the x-axis were ordered by decreasing UMI count (FIG. 7C). Here, in a certain region there was a period of strong decline in the number of molecule counts per cell. This region corresponded to a range of molecule counts between 400 and 1200 UMIs per cell. The next metric used to judge an objective threshold (FIG. 7D) was the molecule count distribution of all cells. Many of the cell barcodes had <650 molecule counts—these cell barcodes most likely represented the background signal of the present MARS-seq data set. The number of cell barcodes with a certain number of molecules decreases with increasing molecule count per cell; through this visualization, natural breakpoints in the distribution that could be used as an objective threshold for filtering and normalization were identified, as these breakpoints mark a change in the data structure and quality, and indicate the transition from background to signal, or from low-quality transcriptomes to high-quality transcriptomes. Here, three notable points were identified (FIG. 7D), which corresponded to molecule counts of 650 (left), 1,050 (middle) and 1,700 (right) per cell. To objectively determine which of these points represented a shift in data quality from low to high quality transcriptomes, a turning point needed to be identified (FIG. 7D). In the density plot (FIG. 7D, top panel), the three lines (left, middle, right) are the breakpoints where the slope of the density function (1st derivative of density, FIG. 7D, middle panel) has a sudden change. On the left line, the downward slope (1st derivative) changes from being very steep to less steep, so that the 2nd derivative is the highest at this point. Similarly, on the middle line, the downward slope changes from less steep to more steep, so the 2nd derivative is the lowest. Based on these observations, the three turning points were identified by the 2nd derivative (FIG. 7D, bottom panel). When a cutoff of 650 was applied, the number of molecule counts per cell was too low and the three DC populations—plasmacytoid DC (pDC) and conventional DC (cDC) subsets cDC1 and cDC2, could not be distinguished by principal component analysis (PCA; FIG. 7E). When a cutoff of 1,700 was applied, the number of cells retained was too low. Therefore, the 1,050 cutoff was an optimal tradeoff between the number of cells analyzed (cells retained after filtering by down sampling normalization) and the number of molecule counts in a cell (gene expression information that remains after discarding molecule counts by down sampling).

To ensure data reproducibility, stability and independence of the chosen molecule cutoff, the initial analyses were stimulated using cutoffs of 650, 1,050, 1,700 and 2,350 molecule counts (FIG. 7E). All four chosen simulation values exhibited the same general data topology if the data were dimensionally-reduced using PCA, thus proving that the biological data structure was robust and independent of filtering thresholds. In addition, the influence of the filtering threshold on the gene loadings within the first two principal components were correlated. Principal component 1 (PC1) of the dataset down-sampled to 1,050 molecule counts was highly correlated with PC1 of the datasets down-sampled to either 650 or 1,700 molecule counts (Pearson=0.996 and 0.999, respectively). The same was true for PC2 (Pearson=0.960 and 0.925, respectively). These results indicated that the chosen filtering cutoff of 1,050 was representative and objectively-derived.

The MARS-seq data obtained in this disclosure were generated by two independent experiments (run1 and run2), which were combined for further data analysis. After normalization, the correlation between the average molecule count of all genes in run1 vs run2 was assessed (FIG. 7F, which shows the high correlation between the average molecular counts in both runs (r=0.994)). When assessing for a batch effect, it is important to ensure that runs do not determine the clustering itself. The t-distributed stochastic neighbor embedding (tSNE) values were plotted (FIG. 7G) (cells of run1 and run2 in equal proportions) together with their density estimates. This analysis showed that the general distribution and, therefore, the clustering was not governed by the run, which is in line with the observation that the present clustering identified biologically reasonable groups that clearly corresponded to the three DC populations (pDC, cDC1 and cDC2) (FIG. 1A). Consequently, the observed clusters were not explained by the variance between the runs, but by biology.

The frequencies of cell types were compared, as determined by the clustering, within the two runs (FIG. 7H). This showed that the ratio between the cells in different clusters was comparable between the two runs. Of note, the ratio does not need to be identical in both runs (46). In addition, this analysis showed that no cluster dominated a single run. Due to the fact that we are taking relatively small samples from a large total population, the frequencies of cell types are expected to show natural variation between runs, which could explain slight shifts in cellular frequencies.

### DISCUSSION

Using unsupervised scmRNAseq and CyTOF analyses, the complexity of the human DC lineage at the single cell level was unraveled, revealing a continuous process of differentiation that starts in the BM with CDP, and diverges at the point of emergence of pre-DC and pDC potentials, culminating in maturation of both lineages in the blood. A previous study using traditional surface marker-based approaches had suggested the presence of a minor pre-DC population in PBMC (32), but the combination of high-dimensional techniques and unbiased analyses employed here shows that this minor population had been markedly underestimated: as the present results reveal a population of pre-DC that overlaps with that observed by Breton and colleagues (32) within the CD117⁺CD303⁻CD141⁻ fraction of PBMC, but accounts for >10 fold the number of cells in peripheral blood than was originally estimated, and is considerably more diverse (FIG. 12C).

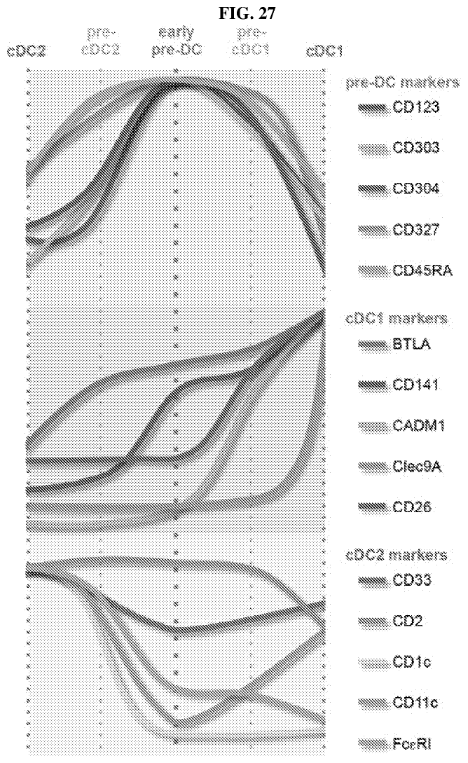

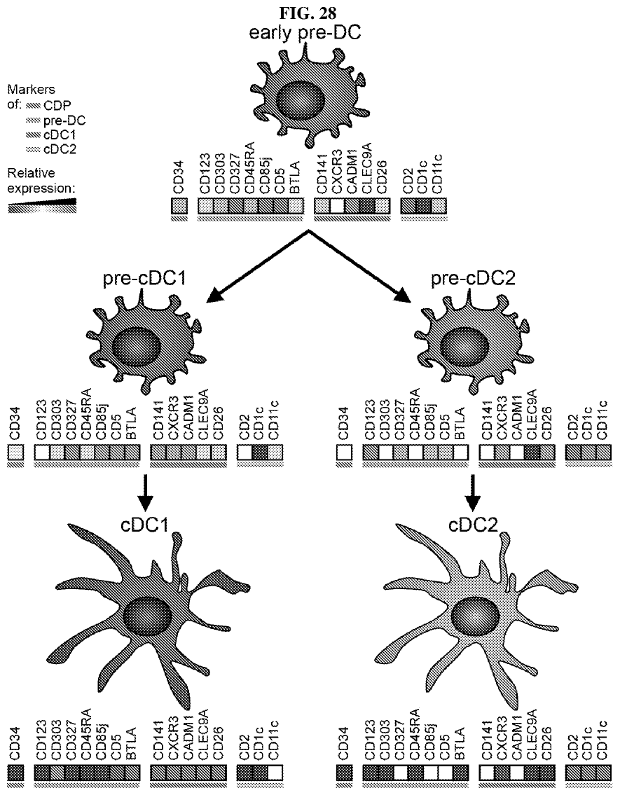

Recent work in mice found uncommitted and subset-committed pre-DC subsets in the BM (38, 43). Here, similarly, three functionally- and phenotypically-distinct pre-DC populations in human PBMC, spleen and BM were identified which are: uncommitted pre-DC and two populations of subset-committed pre-DC (FIG. 27 and FIG. 28). In line with the concept of continuous differentiation from the BM to the periphery, the proportion of uncommitted cells was higher in the pre-DC population in the BM than in the blood. Altogether, these findings support a two-step model of DC development whereby a central transcriptomic subset-specific program is imprinted on DC precursors from the CDP stage onwards, conferring a core subset identity irrespective of the final tissue destination; in the second step of the process, peripheral tissue-dependent programming occurs to ensure site-specific functionality and adaptation (38, 43). Future studies will be required to reveal the molecular events underlying DC subset lineage priming and the tissue-specific cues that regulate their peripheral programming, and to design strategies that specifically target DC subsets at the precursor level. In addition, how the proportions of uncommitted pre-DC versus committed pre-DC are modified in acute and chronic inflammatory settings warrants further investigation.

An important aspect of unbiased analyses is that cells are not excluded from consideration on the basis of preconceptions concerning their surface phenotype. Pre-DC was found to express most of the markers that classically defined pDC, such as CD123, CD303 and CD304. Thus, any strategy relying on these markers to identify and isolate pDC will have inadvertently included CD123⁺CD33⁺ pre-DC as well. While this calls for reconsideration of some aspects of pDC population biology, it may also explain earlier findings including that: pDC cultures possess cDC potential and acquire cDC-like morphology (33, 34), as recently observed in murine BM pDC (48); pDC mediate Th1 immunity through production of IFN-α and IL-12 (33, 49-53); pDC exhibit naïve T-cell allostimulatory capacity (35, 51); and pDC express co-stimulatory molecules and exhibit antigen-presentation/cross-presentation capabilities at the expense of IFN-α secretion (49, 1). These observations could be attributed to the undetected pre-DC in the pDC populations described by these studies, and indeed it has been speculated that the IL-12 production observed in these early studies might be due to the presence of contaminating CD11c⁺cDC (53). The present disclosure addressed this possibility by separating CX3CR1⁺CD33⁺CD123⁺CD303⁺CD304⁺ pre-DC from CX3CR1⁻ CD33⁻CD123⁺CD303⁺CD304⁺“pure” pDC and showing that pDC could not polarize or induce proliferation of naïve CD4 T cells, whereas pre-DC had this capacity; and that pDC were unable to produce IL-12, unlike pre-DC, but were the only cells capable of strongly producing IFN-α in response to TRL7/8/9 agonists, as initially described (54). Thus, it is of paramount importance that pre-DC be excluded from pDC populations in future studies, particularly when using commercial pDC isolation kits. Finally, if pDC are stripped of all their cDC properties, it raises the question as to whether they truly belong to the DC lineage, or rather are a distinct type of innate IFN-I-producing lymphoid cell. It also remains to be shown whether the BM CD34⁺CD123^{hi} CDP population is also a mixture of independent bona fide cDC progenitors and pDC progenitors.

Despite their classification as precursors, human pre-DC appear functional in their own right, being equipped with some T-cell co-stimulatory molecules, and with a strong capacity for naïve T-cell allostimulation and cytokine secretion in response to TLR stimulation (FIG. 2, FIG. 5, FIG. 13, and FIG. 15). Pre-DC produced low levels of IFN-α in response to CpG ODN 2216 exposure, and secreted IL-12 and TNF-α in response to various TLR ligands. Hence, it is reasonable to propose that pre-DC have the potential to contribute to both homeostasis and various pathological processes, particularly inflammatory and autoimmune diseases where dysregulation of their differentiation continuum or their arrested development could render them a potent source of inflammatory DC ready for rapid recruitment and mobilization.

Beyond the identification of pre-DC, the present data revealed previously-unappreciated transcriptional and phenotypic heterogeneity within the circulating mature DC populations. This was particularly clear in the case of cDC2 and pDC, which were grouped into multiple Mpath clusters in the single-cell RNAseq analysis, and showed marked dispersion in the tSNE analysis of the CyTOF data with phenotypic heterogeneity. IsoMAP analysis of the CyTOF data also revealed another level of pDC heterogeneity by illustrating the progressive phenotypic transition from CDP into CD2⁺ pDC in the BM, involving intermediate cells that could be pre-pDC. Whether a circulating pre-pDC population exists remains to be concluded. Finally, defining the mechanisms that direct the differentiation of uncommitted pre-DC into cDC1 or cDC2, or that maintain these cells in their initial uncommitted state in health and disease could lead to the development of new therapeutic strategies to modulate this differentiation process.

In summary, the present invention revealed the complexity of human DC lineage at the single cell level. DC in the bone marrow start as common CDP and diverge at the point of emergence into pre-DC and pDC potentials, culminating in maturation of both lineages in the blood. Furthermore, three functionally and phenotypically distinct pre-DC populations were identified in the human PBMC, spleen and bone marrow: uncommitted pre-DC and two populations of subset-committed pre-DC (pre-cDC1 and pre-cDC2). Importantly, the present invention revealed a novel activation pathway of pre-DC that unlike mature DC subsets, committed pre-DC subsets respond to TLR9 stimulation. PBMC was stimulated with TLR agonists and their cytokine production was measured. Pre-DC produced significantly more TNF-α and IL-12p40 when exposed to CpG ODN 2216 (TLR9 agonist), than to either LPS (TLR4 agonist) or polyI:C (TLR3 agonist) (p=0.03, Mann-Witney test) (FIG. 5). CpG ODN 2216 stimulation also triggered IL-12p40 and TNF-α production by early pre-DC, pre-cDC1, and to a lesser extent pre-cDC2. The application of the TLR9 stimulation of pre-DC may include using a combination of one or more TLR9 agonists (such as CpG) and an antigen delivery system that specifically targets pre-DC and committed pre-DC (for example, by inclusion of an antibody that specifically targets pre-DC and committed pre-DC) to (i) mobilize and activate pre-DC, (ii) deliver the antigen to pre-DC for presentation of antigenic peptides to T cells, and (iii) activate antigen specific T cells. The design strategy could be used in immunotherapy for cancer and other diseases.

### REFERENCES

1. G. Hoeffel et al., Antigen crosspresentation by human plasmacytoid dendritic cells. Immunity. 27, 481-492 (2007).
2. M. Haniffa et al., Human tissues contain CD141hi cross-presenting dendritic cells with functional homology to mouse CD103+ nonlymphoid dendritic cells. Immunity. 37, 60-73 (2012).
3. D. A. Jaitin et al., Massively parallel single-cell RNA-seq for marker-free decomposition of tissues into cell types. Science. 343, 776-779 (2014).
4. B. Langmead, C. Trapnell, M. Pop, S. L. Salzberg, Ultrafast and memory-efficient alignment of short DNA sequences to the human genome. Genome Biol. 10, R25 (2009).
5. D. Grün et al., Single-cell messenger RNA sequencing reveals rare intestinal cell types. Nature. 525, 251-255 (2015).
6. J. Chen, A. Schlitzer, S. Chakarov, F. Ginhoux, M. Poidinger, Mpath maps multi-branching single-cell trajectories revealing progenitor cell progression during development. Nat Commun. 7,11988 (2016).
7. J. Lamb, The Connectivity Map: Using Gene-Expression Signatures to Connect Small Molecules, Genes, and Disease. Science. 313, 1929-1935 (2006).
8. M. Ester, H. P. Kriegel, J. Sander, X. Xu, A density-based algorithm for discovering clusters in large spatial databases with noise. Kdd (1996).
9. M. Hahsler, M. Piekenbrock, dbscan: Density Based Clustering of Applications with Noise (DBSCAN) and Related Algorithms. R package version 1.0-0. https://CRAN.R-project.org/package=dbscan (2017).
10. C. Trapnell et al., The dynamics and regulators of cell fate decisions are revealed by pseudotemporal ordering of single cells. Nat. Biotechnol. 32, 381-386 (2014).
11. M. Setty et al., Wishbone identifies bifurcating developmental trajectories from single-cell data. Nat. Biotechnol. 34, 637-645 (2016).
12. R. R. Coifman et al., Geometric diffusions as a tool for harmonic analysis and structure definition of data: multiscale methods. Proceedings of the National Academy of Sciences. 102, 7432-7437 (2005).
13. J. Harrow et al., GENCODE: the reference human genome annotation for The ENCODE Project. Genome Res. 22, 1760-1774 (2012).
14. B. Li, C. N. Dewey, RSEM: accurate transcript quantification from RNA-Seq data with or without a reference genome. BMC Bioinformatics. 12, 323 (2011).
15. B. Becher et al., High-dimensional analysis of the murine myeloid cell system. Nat. Immunol. 15, 1181-1189 (2014).
16. R. Finck et al., Normalization of mass cytometry data with bead standards. Cytometry A. 83, 483-494 (2013).
17. E. W. Newell, N. Sigal, S. C. Bendall, G. P. Nolan, M. M. Davis, Cytometry by time-of-flight shows combinatorial cytokine expression and virus-specific cell niches within a continuum of CD8+ T cell phenotypes. Immunity. 36, 142-152 (2012).
18. D. R. Parks, M. Roederer, W. A. Moore, A new “Logicle” display method avoids deceptive effects of logarithmic scaling for low signals and compensated data. Cytometry A. 69, 541-551 (2006).
19. L. Van der Maaten, Visualizing data using t-SNE. Journal of Machine Learning Research. 9, 2579-2625 (2008).
20. J. B. Tenenbaum, V. de Silva, J. C. Langford, A global geometric framework for nonlinear dimensionality reduction. Science. 290, 2319-2323 (2000).
21. J. Oksanen et al., vegan: Community Ecology Package. R package version 2.4-2. https://CRAN.R-project.org/package=vegan (2017).
22. J. H. Levine et al., Data-Driven Phenotypic Dissection of AML Reveals Progenitor-like Cells that Correlate with Prognosis. Cell. 162, 184-197 (2015).
23. H. Chen et al., Cytofkit: A Bioconductor Package for an Integrated Mass Cytometry Data Analysis Pipeline. PLoS Comput Biol. 12, e1005112 (2016).
24. J. Lee et al., Restricted dendritic cell and monocyte progenitors in human cord blood and bone marrow. J. Exp. Med. 212, 385-399 (2015).
25. G. K. Smyth, Linear models and empirical bayes methods for assessing differential expression in microarray experiments. Stat Appl Genet Mol Biol. 3, Article 3 (2004).
26. Y. Benjamini, Y. Hochberg, Controlling the false discovery rate: a practical and powerful approach to multiple testing. Journal of the royal statistical society Series B (Methodological) 57, 289-300 (1995).
27. E.-A. D. Amir et al., viSNE enables visualization of high dimensional single-cell data and reveals phenotypic heterogeneity of leukemia. Nat. Biotechnol. 31, 545-552 (2013).
28. C. Sadaka, M.-A. Marloie-Provost, V. Soumelis, P. Benaroch, Developmental regulation of MHC II expression and transport in human plasmacytoid-derived dendritic cells. Blood. 113, 2127-2135 (2009).
29. A. Dzionek et al., BDCA-2, BDCA-3, and BDCA-4: Three Markers for Distinct Subsets of Dendritic Cells in Human Peripheral Blood. The Journal of Immunology. 165, 6037-6046 (2000).
30. S. L. Jongbloed et al., Human CD141+(BDCA-3)+ dendritic cells (DCs) represent a unique myeloid DC subset that cross-presents necrotic cell antigens. J. Exp. Med. 207, 1247-1260 (2010).
31. K. P. A. MacDonald et al., Characterization of human blood dendritic cell subsets. Blood. 100, 4512-4520 (2002).
32. G. Breton et al., Circulating precursors of human CD1c+ and CD141+ dendritic cells. J. Exp. Med. 212, 401-413 (2015).
33. M. Cella et al., Plasmacytoid monocytes migrate to inflamed lymph nodes and produce large amounts of type I interferon. Nat. Med. 5, 919-923 (1999).
34. G. Grouard et al., The enigmatic plasmacytoid T cells develop into dendritic cells with interleukin (IL)-3 and CD40-ligand. J. Exp. Med. 185, 1101-1111 (1997).
35. T. Matsui et al., CD2 distinguishes two subsets of human plasmacytoid dendritic cells with distinct phenotype and functions. J. Immunol. 182, 6815-6823 (2009).
36. H. Yu et al., Human BDCA2(+)CD123 (+)CD56 (+) dendritic cells (DCs) related to blastic plasmacytoid dendritic cell neoplasm represent a unique myeloid DC subset. Protein Cell. 6, 297-306 (2015).
37. B. Reizis, A. Bunin, H. S. Ghosh, K. L. Lewis, V. Sisirak, Plasmacytoid dendritic cells: recent progress and open questions. Annu. Rev. Immunol. 29, 163-183 (2011).
38. A. Schlitzer et al., Identification of cDC1- and cDC2-committed DC progenitors reveals early lineage priming at the common DC progenitor stage in the bone marrow. Nat. Immunol. 16, 718-728 (2015).
39. S. C. Bendall et al., Single-cell trajectory detection uncovers progression and regulatory coordination in human B cell development. Cell. 157, 714-725 (2014).
40. M. Merad, P. Sathe, J. Helft, J. Miller, A. Mortha, The dendritic cell lineage: ontogeny and function of dendritic cells and their subsets in the steady state and the inflamed setting. Annu. Rev. Immunol. 31, 563-604 (2013).
41. M. Guilliams et al., Dendritic cells, monocytes and macrophages: a unified nomenclature based on ontogeny. Nat. Rev. Immunol. 14, 571-578 (2014).
42. A. Schlitzer, N. McGovern, F. Ginhoux, Dendritic cells and monocyte-derived cells: Two complementary and integrated functional systems. Semin. Cell Dev. Biol. 41, 9-22 (2015).
43. M. Swiecki, M. Colonna, The multifaceted biology of plasmacytoid dendritic cells. Nat. Rev. Immunol. 15, 471-485 (2015).
44. E. Mass et al., Specification of tissue-resident macrophages during organogenesis. Science. 353 (2016), doi:10.1126/science.aaf4238.
45. G. X. Y. Zheng et al., Massively parallel digital transcriptional profiling of single cells. Nat Commun. 8, 14049 (2017).
46. F. Paul et al., Transcriptional Heterogeneity and Lineage Commitment in Myeloid Progenitors. Cell. 163, 1663-1677 (2015).
47. O. Matcovitch-Natan et al., Microglia development follows a stepwise program to regulate brain homeostasis. Science. 353, aad8670 (2016).
48. A. Schlitzer et al., Identification of CCR9-murine plasmacytoid DC precursors with plasticity to differentiate into conventional DCs. Blood. 117, 6562-6570 (2011).
49. A. Krug et al., Toll-like receptor expression reveals CpG DNA as a unique microbial stimulus for plasmacytoid dendritic cells which synergizes with CD40 ligand to induce high amounts of IL-12. Eur. J. Immunol. 31, 3026-3037 (2001).
50. A. Dzionek et al., Plasmacytoid dendritic cells: from specific surface markers to specific cellular functions. Hum. Immunol. 63, 1133-1148 (2002).
51. M. Cella, F. Facchetti, A. Lanzavecchia, M. Colonna, Plasmacytoid dendritic cells activated by influenza virus and CD40L drive a potent TH1 polarization. Nat. Immunol. 1, 305-310 (2000).
52. T. Ito et al., Plasmacytoid dendritic cells prime IL-10-producing T regulatory cells by inducible costimulator ligand. J. Exp. Med. 204, 105-115 (2007).
53. J.-F. Fonteneau et al., Activation of influenza virus-specific CD4+ and CD8+ T cells: a new role for plasmacytoid dendritic cells in adaptive immunity. Blood. 101, 3520-3526 (2003).
54. F. P. Siegal et al., The Nature of the Principal Type 1 Interferon-Producing Cells in Human Blood. Science. 284, 1835-1837 (1999).
55. IPA's Upstream Regulator Analysis Validation Whitepaper: A Novel Approach to Predicting Upstream Regulators. http://pages.ingenuity.com/IPAUpstreamRegulatorAnalysisValidationWP.html.
56. Ingenuity Downstream Effects Analysis in IPA Whitepaper: Identify Biological Functions That Are Expected To Be Increased Or Decreased Given The Observed Gene Expression with IPA. http://pages.ingenuity.com/IngenuityDownstreamEffectsAnalysisinIPAWhitepaper.html
57. B. Cisse et al., Transcription factor E2-2 is an essential and specific regulator of plasmacytoid dendritic cell development. Cell. 135, 37-48 (2008).

1.-43. (canceled)

44. A method of treating or preventing an infection, a neoplastic disease or an immune-related disease in a subject in need thereof, the method comprising contacting a therapeutically effective or immuno-effective amount of an TLR9 agonist with a precursor dendritic cell (pre-DC), wherein the TLR9 agonist stimulates the pre-DC to secrete one or more cytokines, to thereby activate or increase the subject's immune response for treating or preventing the infection, the neoplastic disease or the immune-related disease.

45. The method of claim 44, wherein the contacting is one or more of the following:
(a) a contacting carried out in vitro, in vivo or ex vivo; and
(b) a contacting including administering by a route selected from the group consisting of intramuscular, intradermal, subcutaneous, intravenous, oral, topical and intranasal administration.

46. The method of claim 44, wherein the pre-DC is one or more of the following:
(a) a pre-DC that presents an antigen (or a fragment thereof) associated with the infection, the neoplastic disease or the immune related disease;
(b) a pre-DC selected from the group consisting of early pre-DC, pre-conventional dendritic cells 1 (pre-cDC1), and pre-conventional dendritic cells 2 (pre-cDC2); and
(c) a pre-DC comprising one or more markers selected from the group consisting of CD123, CD303, CD304, CD327, CD45RA, CD85j, CD5 and BTLA.

47. The method of claim 44, wherein the method comprises one or more of the following:
(a) a method wherein the infection is selected from the group consisting of a bacterial infection and a viral infection;
(b) a method wherein the immune-related disease is an inflammatory disease or an autoimmune disease; and
(c) a method wherein the autoimmune disease is selected from the group consisting of systemic lupus erythematosus (SLE) and Sjögren's syndrome.

48. The method of claim 44, wherein the one or more TLR9 agonists is one or more of the following:
(a) an oligodeoxynucleotide;
(b) an oligodeoxynucleotide selected from the group consisting of CpG oligodeoxynucleotide (ODN) Class A, CpG ODN Class B and CpG ODN Class C; (c) a CpG ODN Class A which is CpG ODN 2216; and
(d) a vaccine.

49. The method of claim 44, wherein the method comprises one or more of the following:
(a) a method further comprising using an antigen delivery system that specifically targets pre-DC and committed pre-DC;
(b) a method wherein the antigen delivery system comprises an antibody that specifically targets pre-DC and committed pre-DC;
(c) a method wherein the one or more cytokine is selected from the group consisting of interferons, tumor necrosis factors, interleukins, and chemokines;
(d) a method wherein the interferon is IFN-α; the tumor necrosis factor is TNF-α; and the interleukin is IL-12p40; and
(e) a method wherein the subject is a human.

50. An immunogenic composition comprising one or more TLR9 agonists capable of stimulating pre-DC to secrete one or more cytokines.

51. The immunogenic composition of claim 50, further comprising one or more of the following:
(a) an antigen (or a fragment thereof) associated with an infection, a neoplastic disease or an immune-related disease;
(b) an antigen delivery system that specifically targets pre-DC and committed pre-DC; and
(c) an adjuvant, a preservative, a stabilizer, an encapsulating agent (e.g. lipid membranes, chitosan particles, biocompatible polymers) and/or a pharmaceutically acceptable carrier.

52. The immunogenic composition of claim 51, wherein the antigen delivery system comprises an antibody that specifically targets pre-DC and committed pre-DC.

53. An adjuvant composition comprising a TLR9 agonist that is capable of stimulating pre-DC to secrete one or more cytokines for activating or increasing a subject's immune response to treat or prevent an infection, a neoplastic disease or an immune-related disease.

54. The adjuvant composition of claim 53, further comprising one or more of the following:
(a) an antigen (or a fragment thereof) associated with an infection, a neoplastic disease or an immune-related disease; and
(b) an antigen delivery system that specifically targets pre-DC and committed pre-DC.

55. The adjuvant composition of claim 54, wherein the antigen delivery system comprises an antibody that specifically targets pre-DC and committed pre-DC.
